(19) United States
(12) Patent Application Publication
(10) Pub. No.: US 2022/0354935 A1
(43) Pub. Date: Nov. 10, 2022

(54) VACCINATION AGAINST FUNGAL EPITOPES TO PREVENT INFLAMMATORY BOWEL DISEASES

(71) Applicants: Lundquist Institute for Biomedical Innovation at Harbor-UCLA Medical Center, Torrance, CA (US); The University of Utah, Salt Lake City, UT (US)

(72) Inventors: Ashraf S. IBRAHIM, Torrance, CA (US); June L. ROUND, Salt Lake City, UT (US); Kyla OST, Salt Lake City, UT (US)

(21) Appl. No.: 17/726,481

(22) Filed: Apr. 21, 2022

Related U.S. Application Data
(60) Provisional application No. 63/177,740, filed on Apr. 21, 2021.

(51) Int. Cl. A61K 39/00; A61K 9/00; A61P 1/00

(52) U.S. Cl. CPC A61K 39/0002; A61K 9/0073; A61P 1/00; A61K 9/0019; A61K 2039/545

(57) ABSTRACT

The invention provides a vaccine against inflammatory bowel disease (IBD), such as Crohn's disease, ulcerative colitis, and the like. The vaccine comprises a polypeptide comprising a Candida adhesin antigen, typically an isolated agglutinin-like sequence (Als) protein antigen, formulated with one or more pharmaceutically acceptable carriers or excipients.

### RELATED PATENT APPLICATIONS

This patent application claims the benefit of U.S. Provisional Patent Application No. 63/177,740 filed on Apr. 21, 2021, entitled VACCINATION AGAINST FUNGAL EPITOPES TO PREVENT INFLAMMATORY BOWEL DISEASES, naming Ashraf S. Ibrahim, June L. Round and Kyla Ost as inventors, and designated by Attorney Docket No. 022098-0560931. The entire content of the foregoing application is incorporated herein by reference, including all text, tables and drawings.

### GOVERNMENT SUPPORT

This invention was made with government support under NIH Grant No. R01 DK 124336-03. The government has certain rights in the invention.

### SEQUENCE LISTING

This application contains a Sequence Listing which has been submitted electronically in ASCII format and is hereby incorporated by reference in its entirety. Said ASCII copy is named “022098-0568730_Sequence_Listing_ST25.txt”, was created on May 12, 2022, and is 12,969 bytes in size.

### LENGTHY TABLE

The present application contains a lengthy table section. The lengthy tables in the application are those referred to as “Supplementary Table 1”, “Supplementary Table 2” and “Supplementary Table 3”. Copies of the tables are available in electronic form from the USPTO web site. Electronic copy of the tables will also be available from the USPTO upon request and payment of the fee set forth in 37 CFR 1.19 (b)(3).

### BACKGROUND OF THE INVENTION

The present invention relates generally to compositions and methods for preventing or treatment for inflammatory bowel diseases in a patient, and more specifically to compositions and methods that induce a protective or therapeutic response against fungi-associated damage during colitis.

Inflammatory bowel diseases (IBDs), principally ulcerative colitis (UC) and Crohn's disease (CD), are inflammatory disorders of the gastrointestinal tract caused by multiple genetic and environmental factors. Patients may have diarrhea, nauseas, vomiting, abdominal cramps, and pain that can be difficult to control. IBDs are characterized by chronic excessive destruction of the colon (in UC) or the small and large bowel (in CD), due to the infiltration of the bowel wall by inflammatory infiltrate. Approximately 1.6 million Americans currently have IBD (as many as 70,000 new cases of IBD are diagnosed in the United States each year), which exerts a significant health and quality of life burden on patients. Surgical intervention can be curative in ulcerative colitis but there is currently no cure for Crohn's disease. Since patients are often diagnosed at a young age, IBD generates a significant burden on the health care system.

The pathogenesis of IBD involves interactions among local environment microorganisms, genetic susceptibility and the immune system. The intestinal microbiota harbors a number of potentially pathogenic fungal commensals. Certain intestinal fungi also induce inflammatory immune responses that exacerbate diseases such as IBD. Crohn's disease is strongly associated with serum anti-Saccharomyces antibodies (ASCAs) that target fungal cell wall components in Saccharomyces and Candida species that dominate the mammalian intestinal fungal community. However, commensal fungi are benign in most healthy individuals. The forces that maintain homeostatic interactions between fungi and host immunity within their mucosal niche are not well defined.

IgA is one of the main effector molecules produced by the intestinal adaptive immune system and multiple studies have demonstrated the importance of IgA in the maintenance of homeostasis with bacteria. While systemic anti-fungal antibody responses have been documented in detail, little is known about how antibodies directly regulate fungi within their commensal niche. Despite the clinical use of ASCAs, little is known about intestinal IgA reactivity against fungi.

The available medical treatments for IBD are rather unsatisfactory. Current medical treatments for IBD rely on the use of non-specific anti-inflammatory drugs such as corticosteroids, as well as immunosuppressive drugs. However, these treatments do not modify the disease course but only ameliorate the symptoms, while inducing severe side effects that limit their use. Moreover, significant percentage of the patients are steroid resistance. Currently there are no preventatives for IBD.

Therefore, there exists a high unmet medical need for development of a vaccine to provide adequate protection against IBD. The present invention satisfies this need and provides related advantages as well.

### SUMMARY OF INVENTION

In accordance with the present invention, there are provided methods of ameliorating and/or preventing an intestinal disease in a mammal comprising administering to the mammal an immunogenic amount of a vaccine comprising a Candida adhesin polypeptide, or an immunogenic fragment thereof, in a pharmaceutically acceptable medium.

The invention also provides vaccines comprising a Candida adhesin polypeptide, or an immunogenic fragment thereof, for use in a method of ameliorating and/or preventing an intestinal disease in a mammal.

### BRIEF DESCRIPTION OF THE DRAWINGS

FIGS. 1A-1K show that adaptive immune responses target and suppress C. albicans hyphae in the gut. P values calculated using two-way ANOVA with Tukey's test (FIG. 1B) or Sidak's test (FIGS. 1C, 1F, and 1I), one-way ANOVA with Tukey's test (FIGS. 1D and 1E), Friedman test with paired Dunn's multiple comparisons (FIG. 1A), two-sided unpaired t-test (FIG. 1J), Wald's test with multiple test correction (FIG. 1G) or the ‘fgsea’ R package that corrects for multiple tests (FIG. 1H). Data are mean±s.d. (FIGS. 1C-1F, 1I and 1J).

FIG. 1A shows human fecal IgA binding to cultured fungi quantified by flow cytometry (n=30 healthy, n=23 Crohn's disease (CD), and n=17 ulcerative colitis (UC)). AU means arbitrary units.

FIG. 1B shows IgA binding to faecal fungi (GFP-yeast, iRFP⁻, CFW⁺) (n=4 S. cerevisiae (Sc)-colonized and n=3 C. albicans (Ca)-colonized mice per group).

FIG. 1C shows IgA per mg of intestinal contents, assessed by enzyme-linked immunosorbent assay (ELISA) in small intestine (SI), caecum and colon from germ-free (GF) or monocolonized mice four weeks after inoculation.

FIG. 1D shows colon lamina propria IgA+ plasma cells (PCs) (IgA⁺CD138⁺CD45⁺CD3⁻CD19⁻ live cells) from monocolonized mice four weeks after inoculation (n=4 mice per group).

FIG. 1E shows Peyer's patch GC B cells (GL-7+Fas+IgD− CD19+ live cells) from monocolonized mice four weeks after inoculation (n=4 mice per group).

FIG. 1F shows intestinal IgA reactivity to cultured C. albicans or S. cerevisiae quantified by flow cytometry (n=4 GF, n=4 C. albicans-colonized and n=4 S. cerevisiae-colonized mice per group). Data in 1B-1F are representative of two experiments.

FIG. 1G shows the volcano plot of the ratio of caecal C. albicans transcripts in monocolonized wild-type (WT) and Rag1^{-/-} mice four weeks after inoculation. SAP4 (Padj=6.43×10⁻⁷³) excluded from plot to better visualize other transcripts.

FIG. 1H shows gene set enrichment analysis (GSEA) of genes upregulated in hyphae from a previous study, described in Witchley, J. N. et al. Candida albicans morphogenesis programs control the balance between gut commensalism and invasive infection, Cell Host Microbe 25, 432-443 (2019)), which is incorporated by reference herein in its entirety.

FIG. 11 shows anti-C. albicans antibody (green) staining of C. albicans in antibiotic-treated wild-type and Rag1^{-/-} mice four weeks after inoculation. Images from caecum (n=5 mice per group; one experiment).

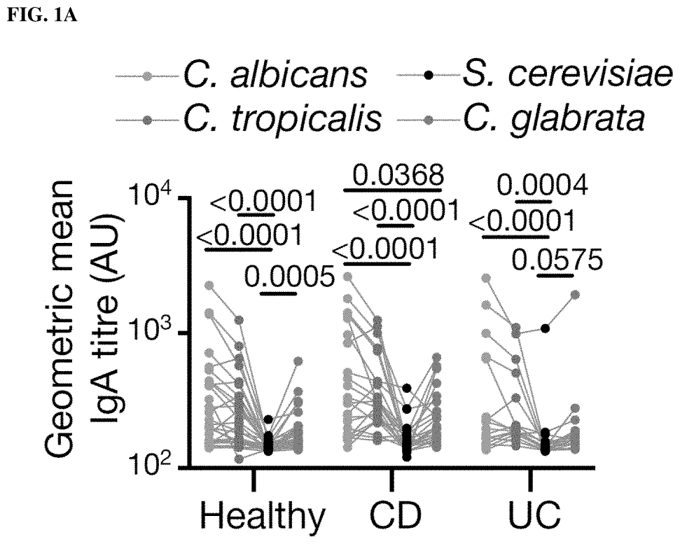

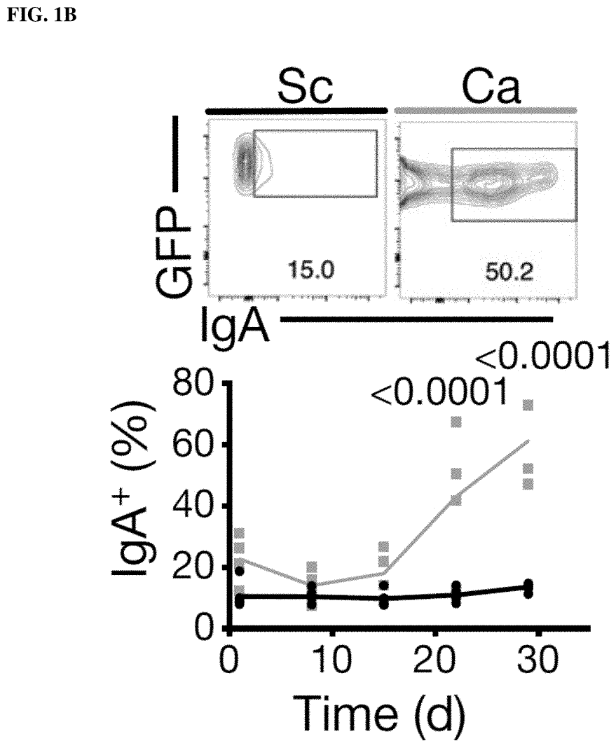

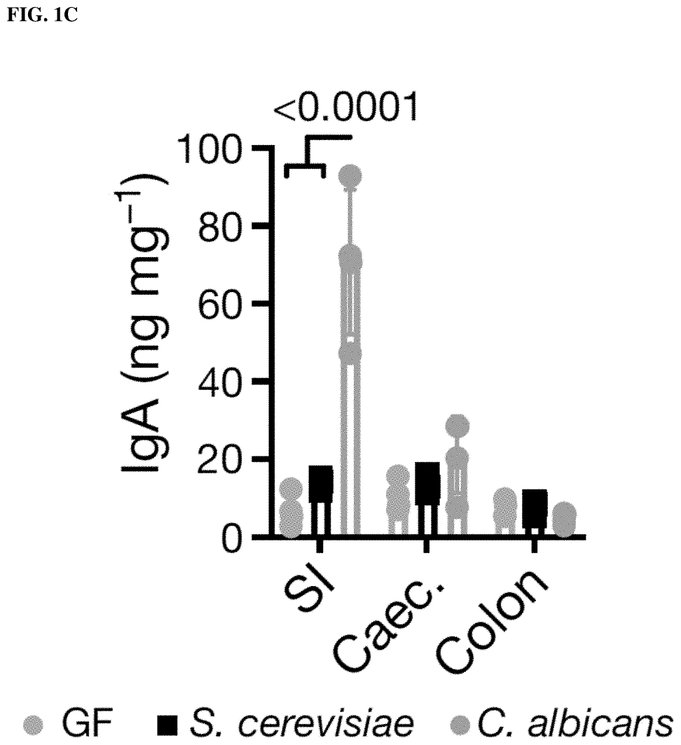

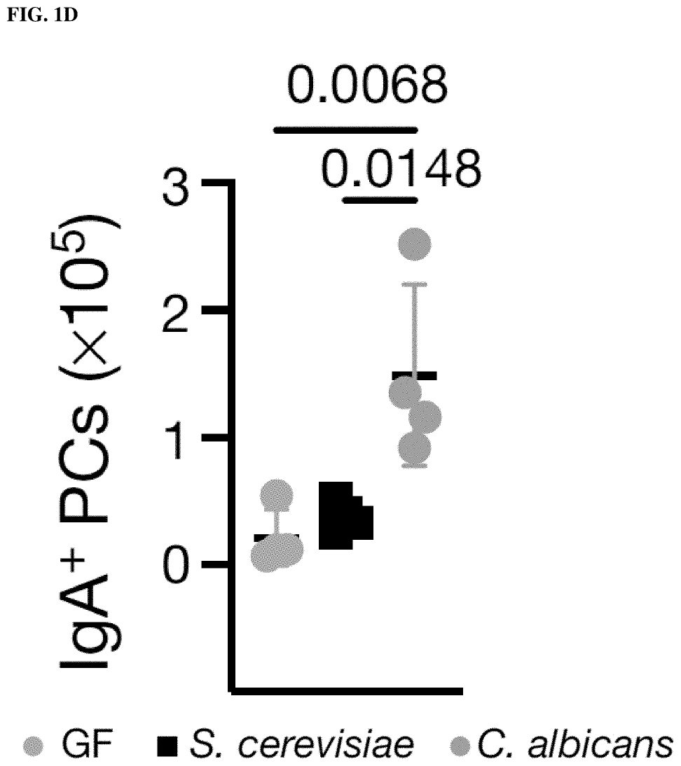

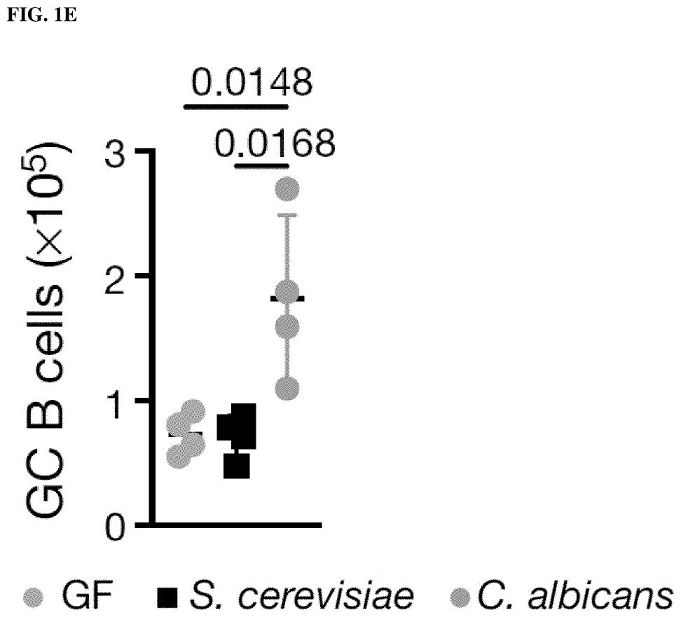

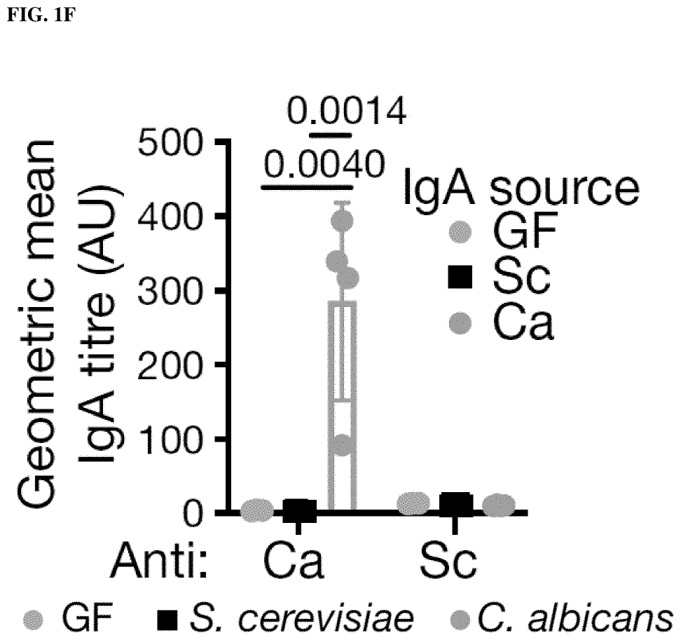

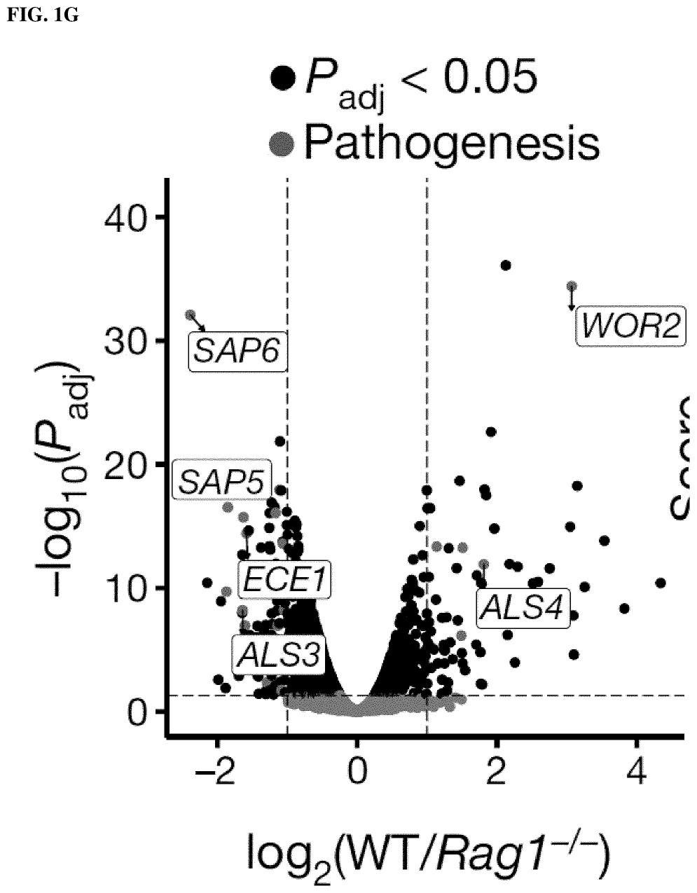

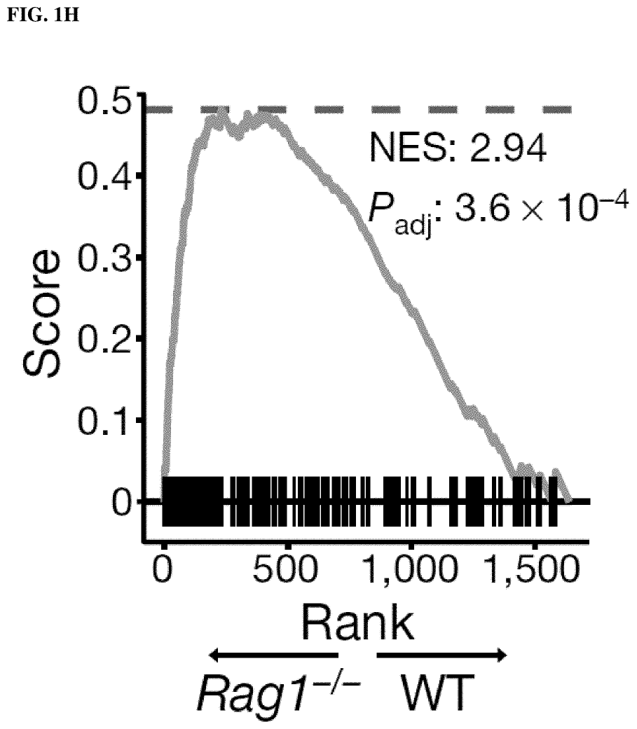

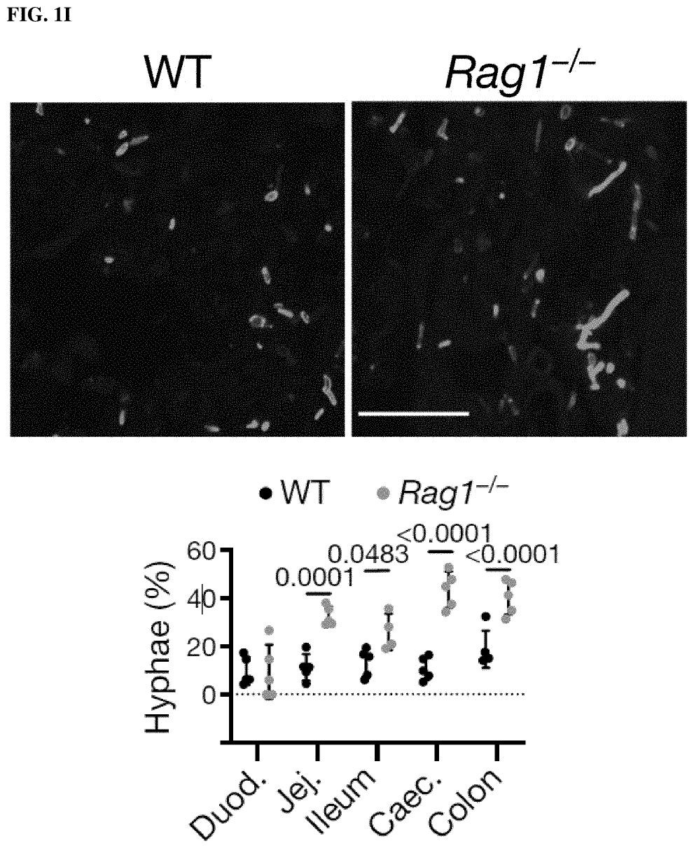

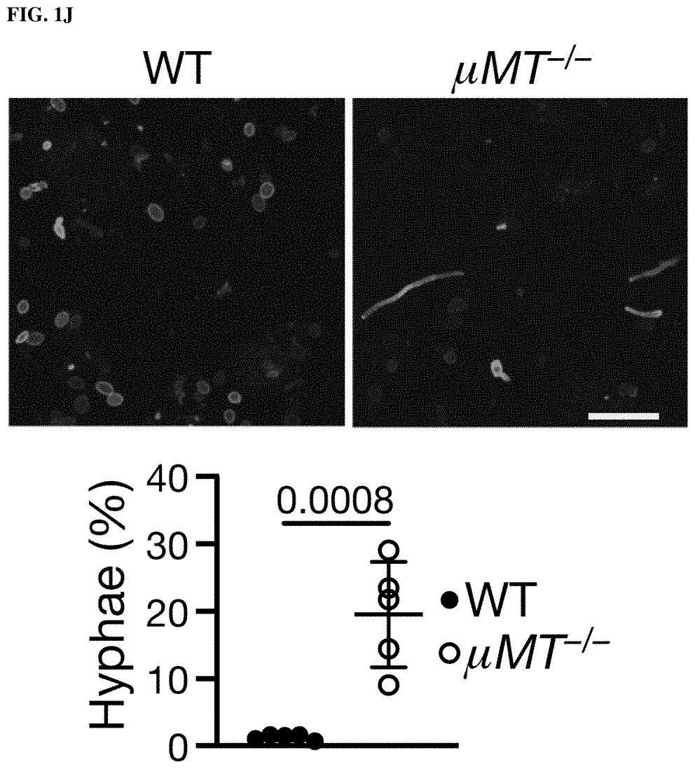

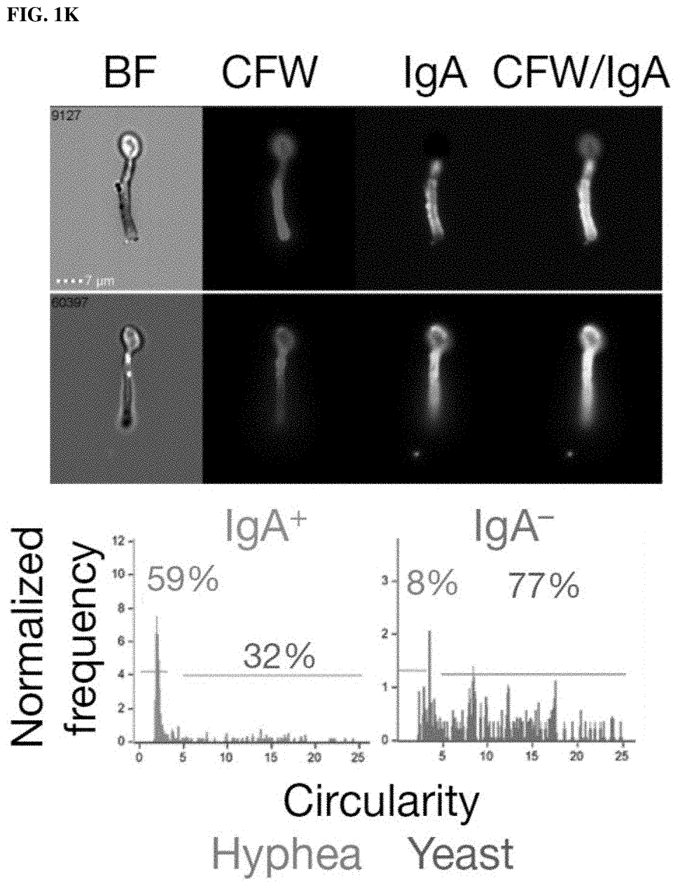

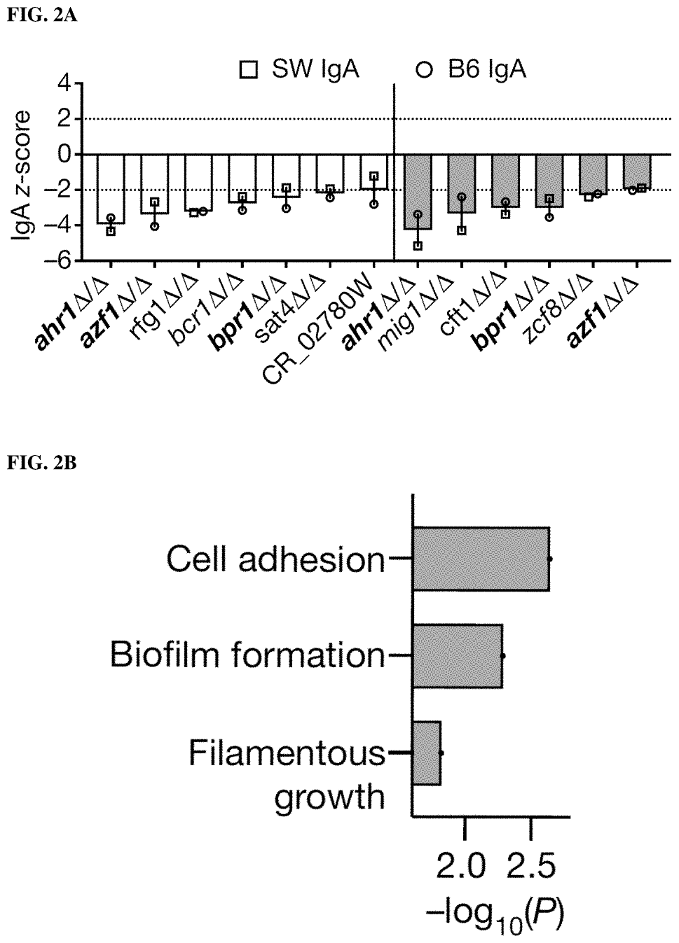

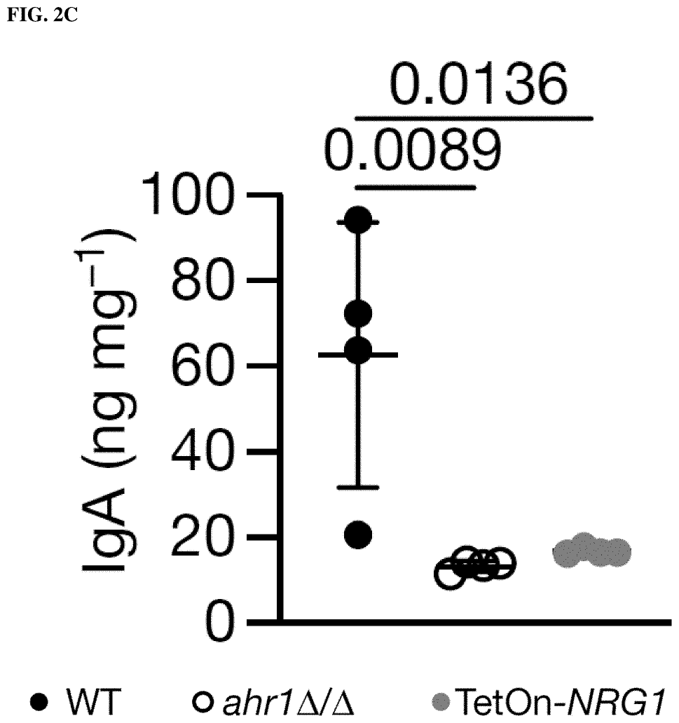

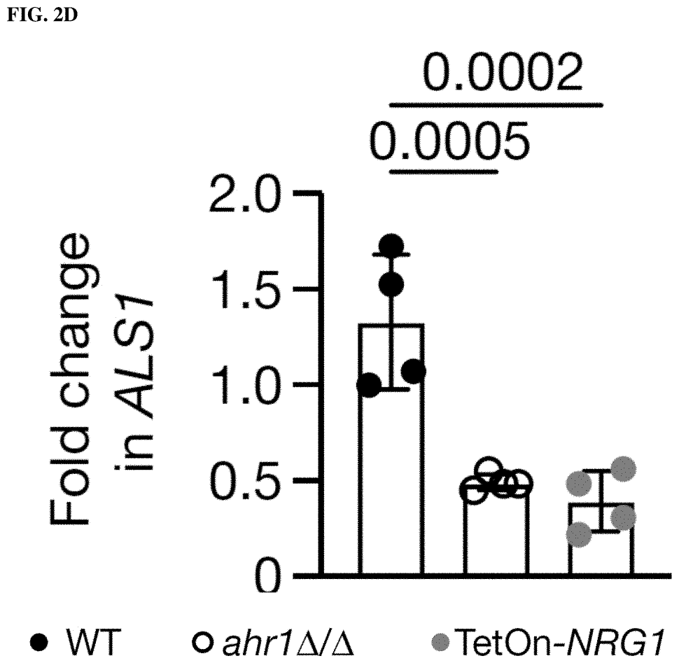

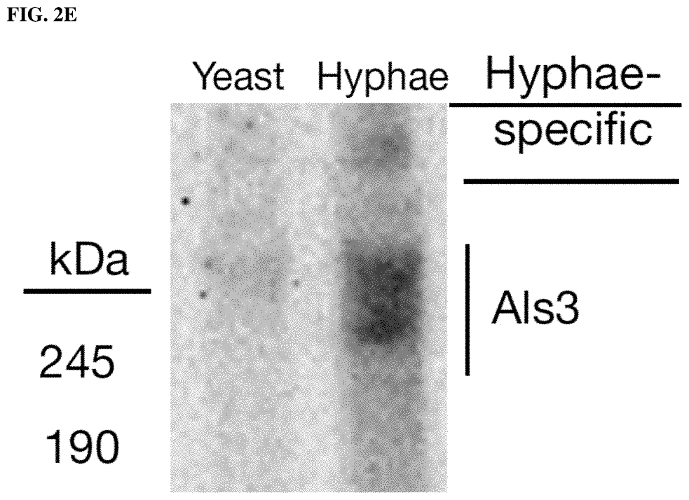

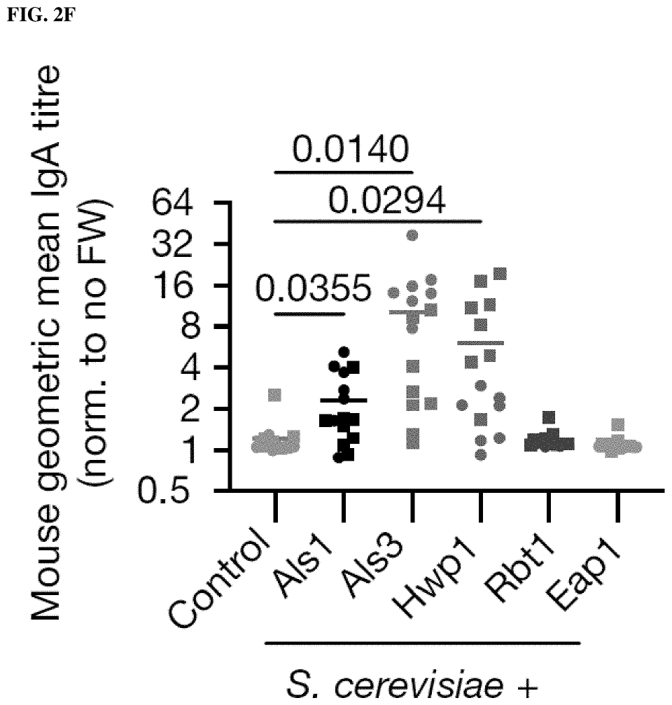

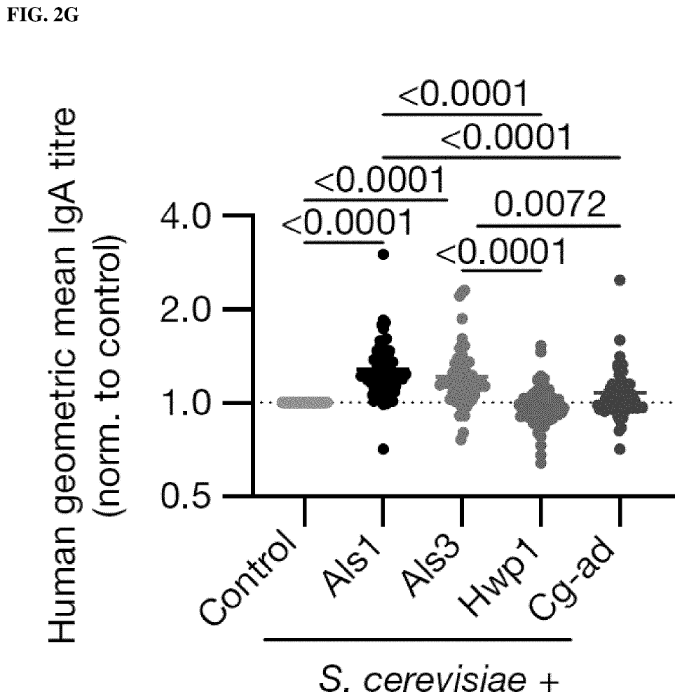

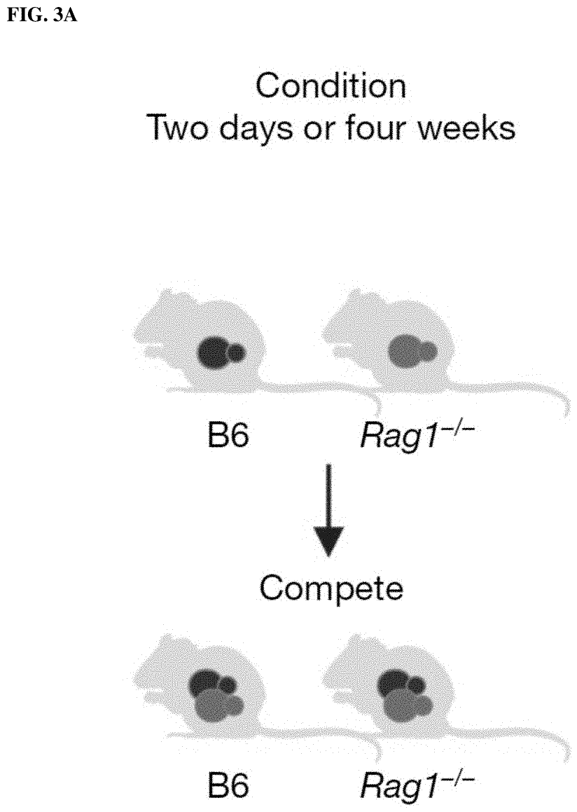

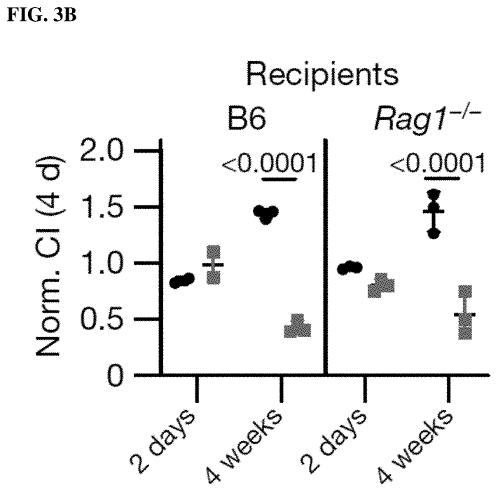

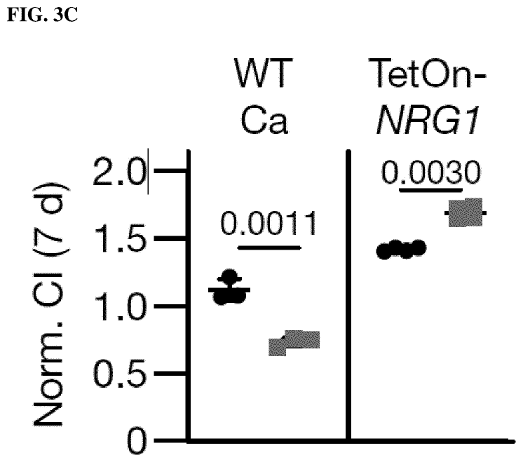

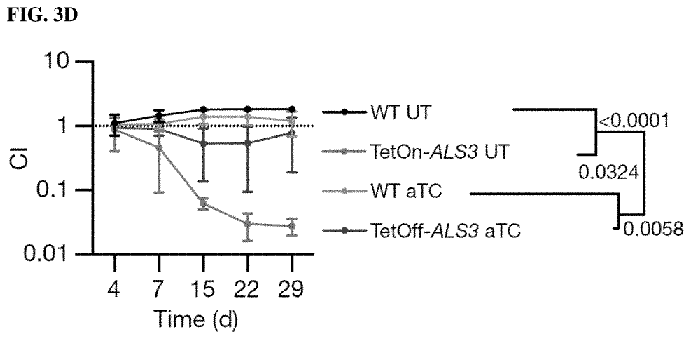

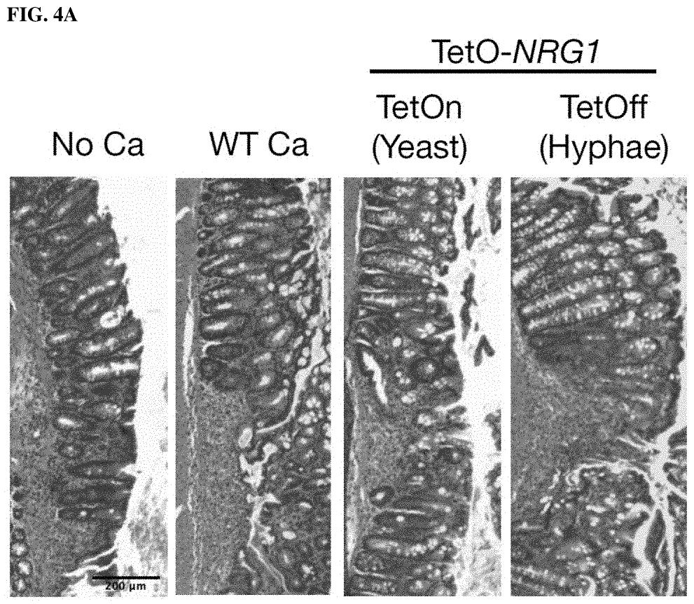

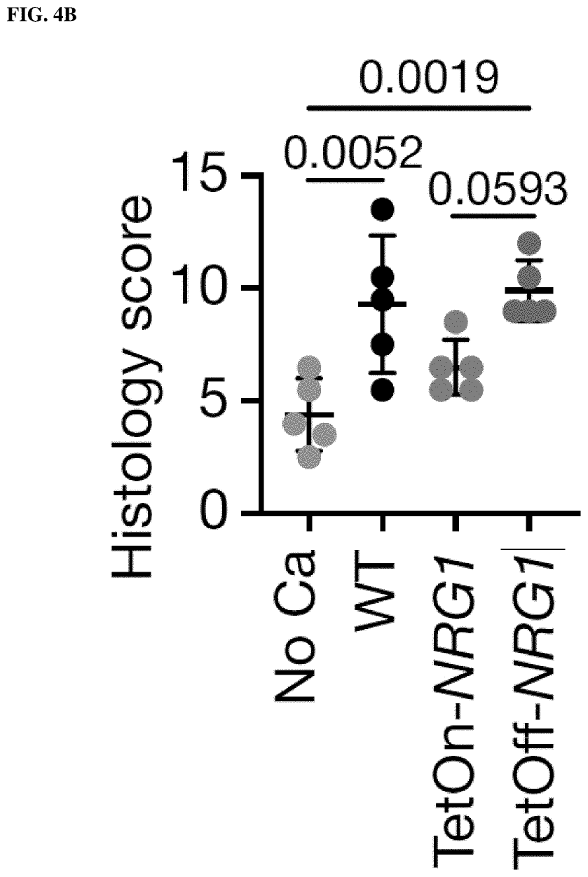

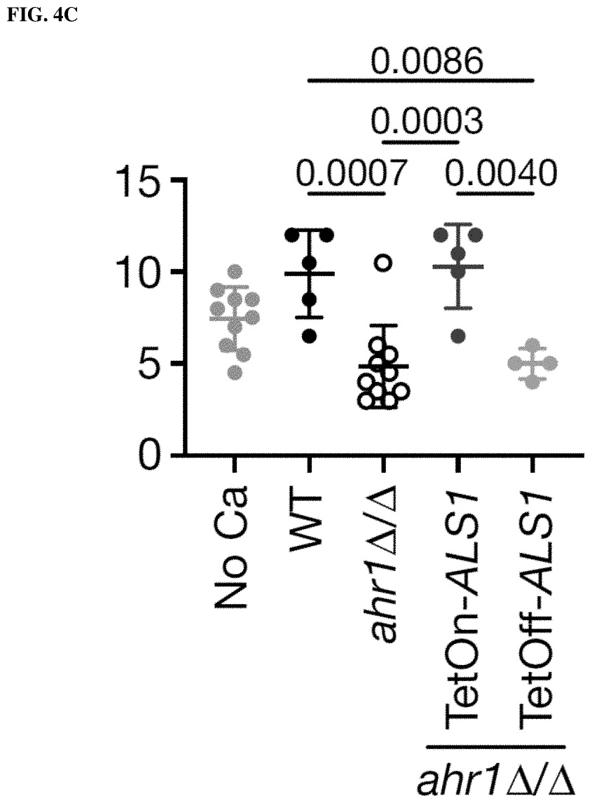

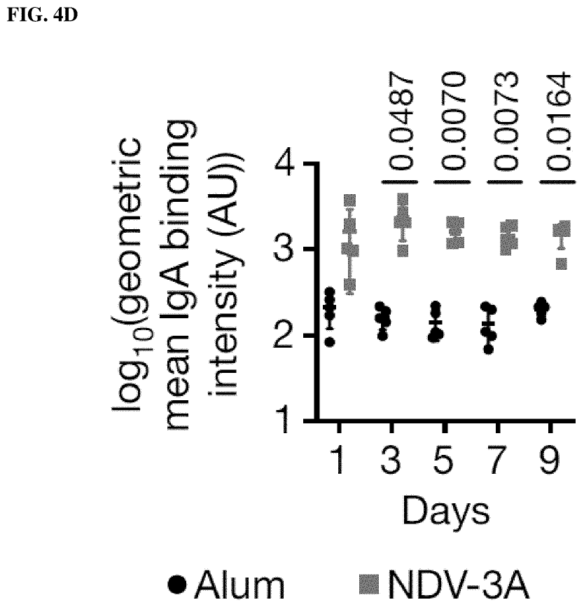

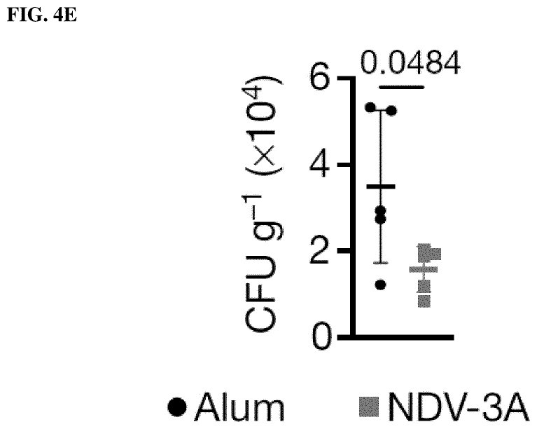

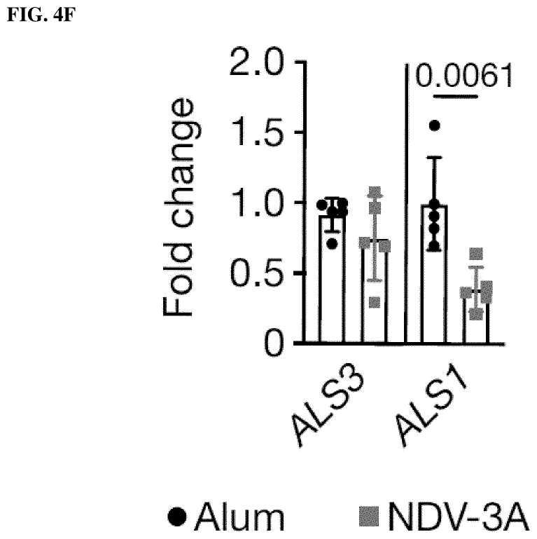

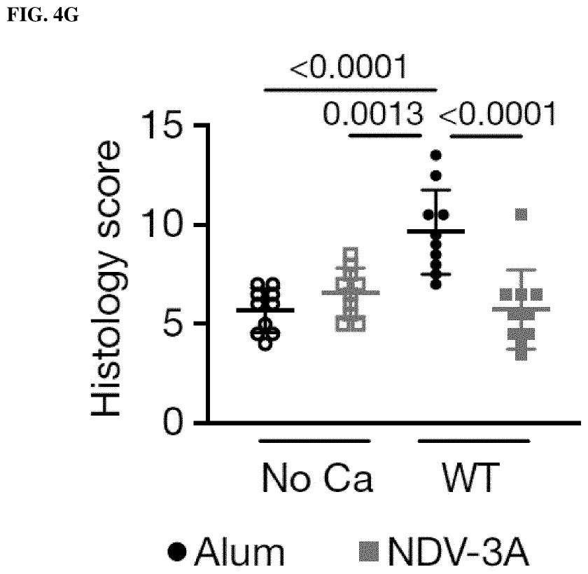

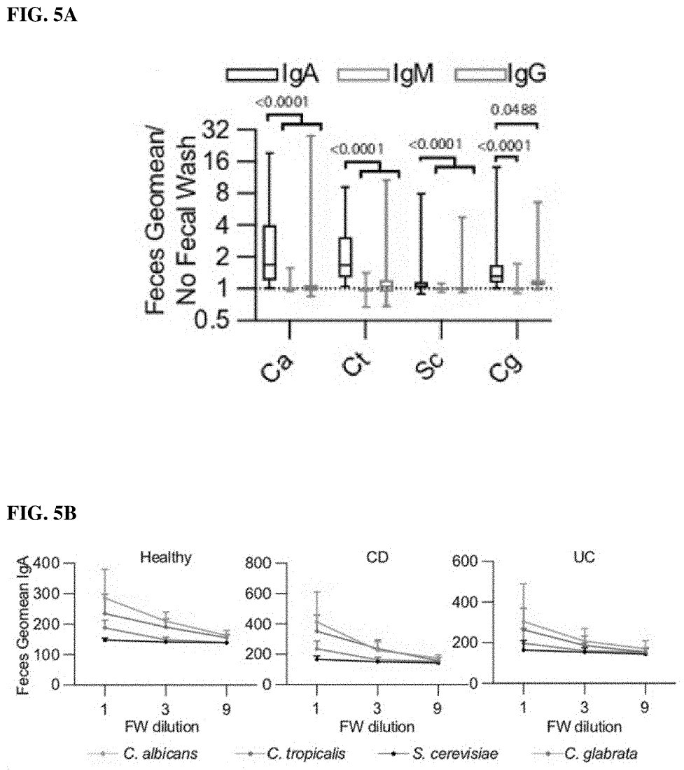

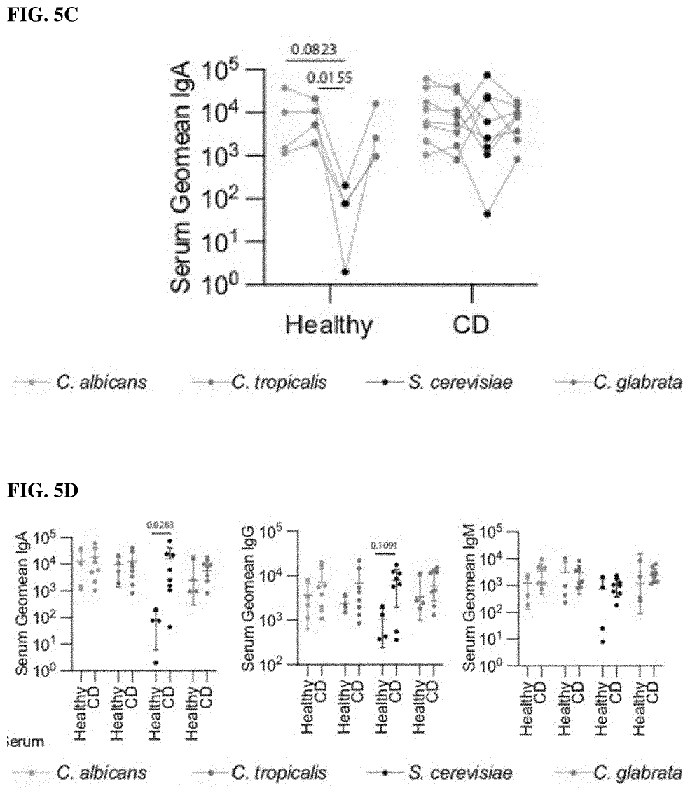

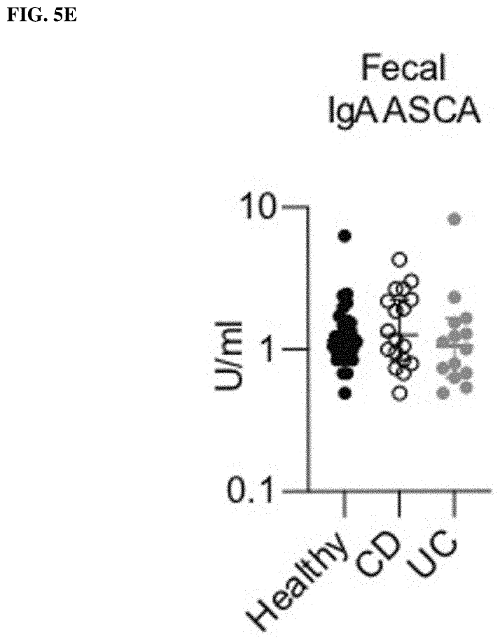

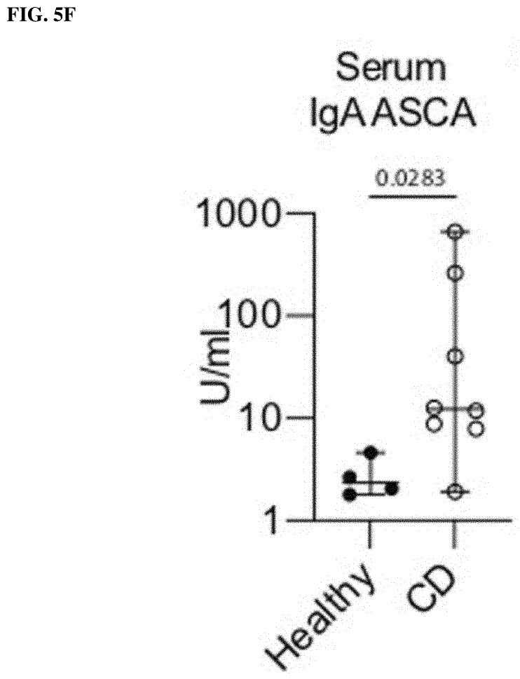

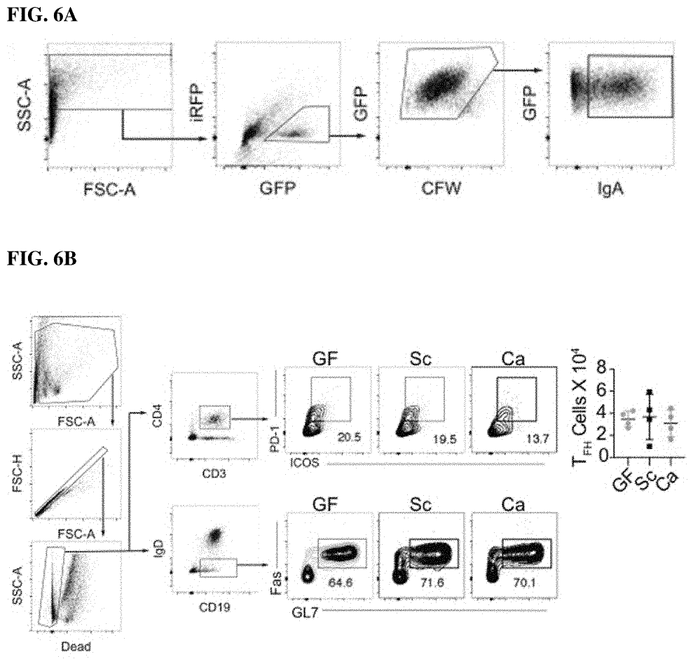

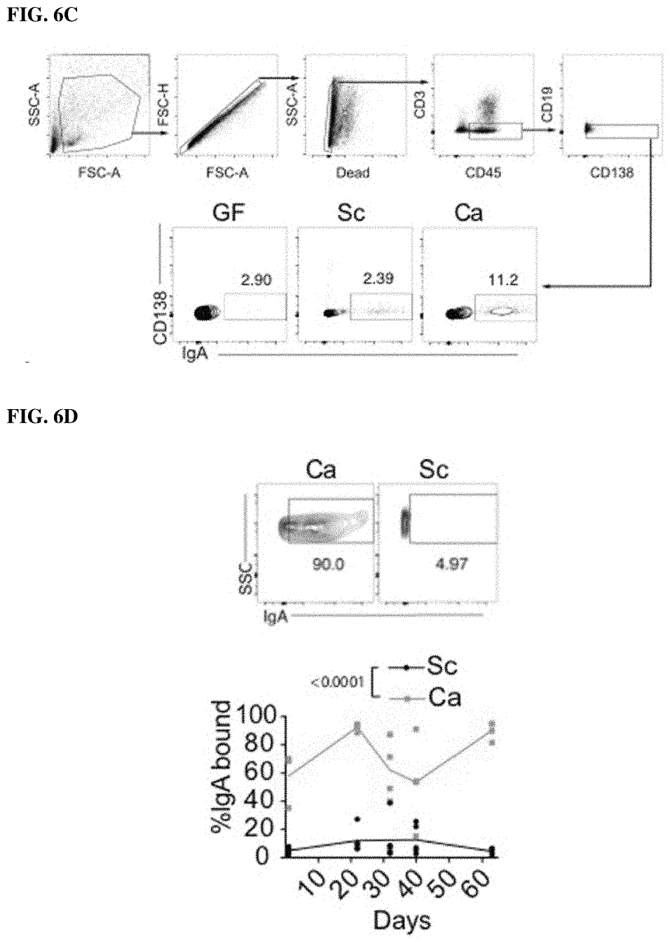

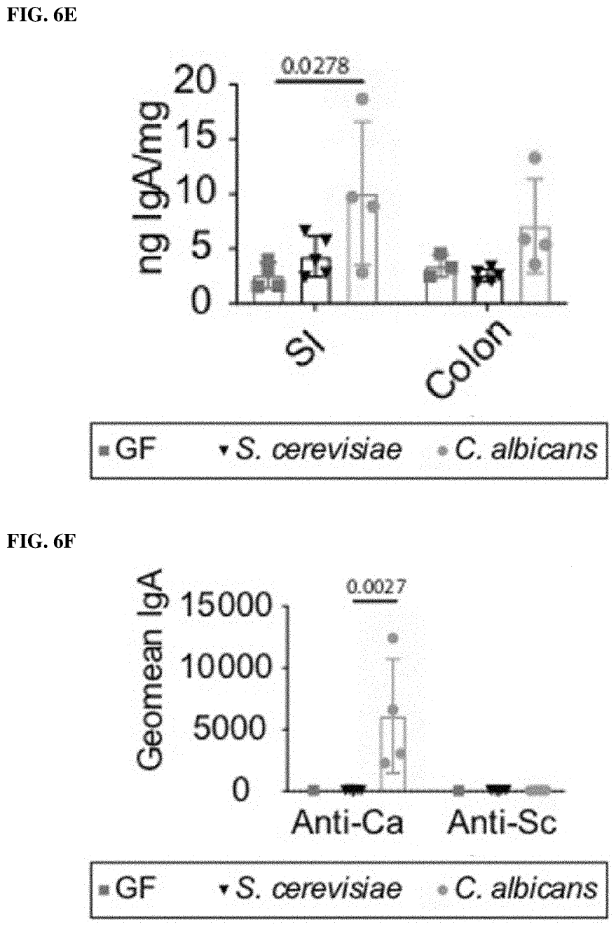

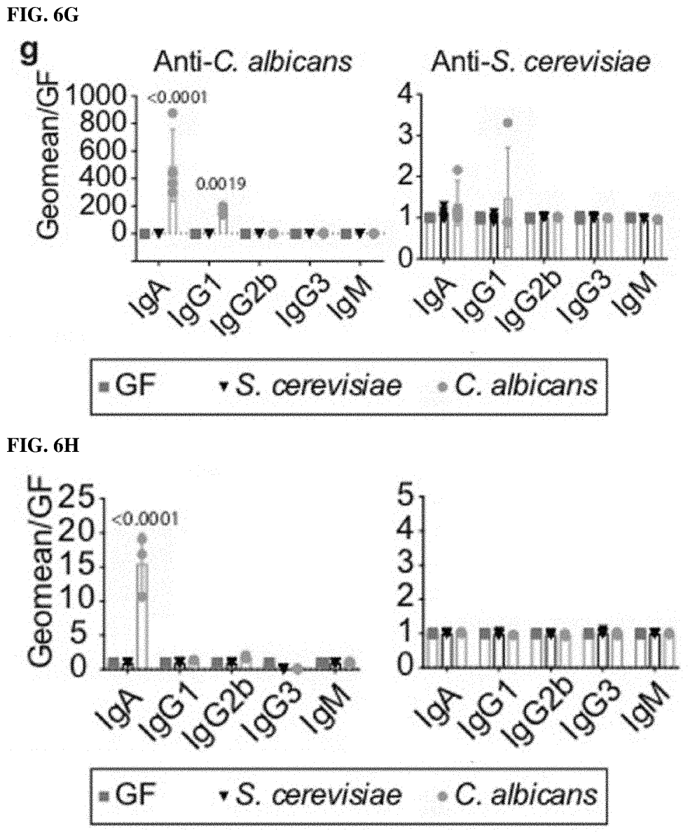

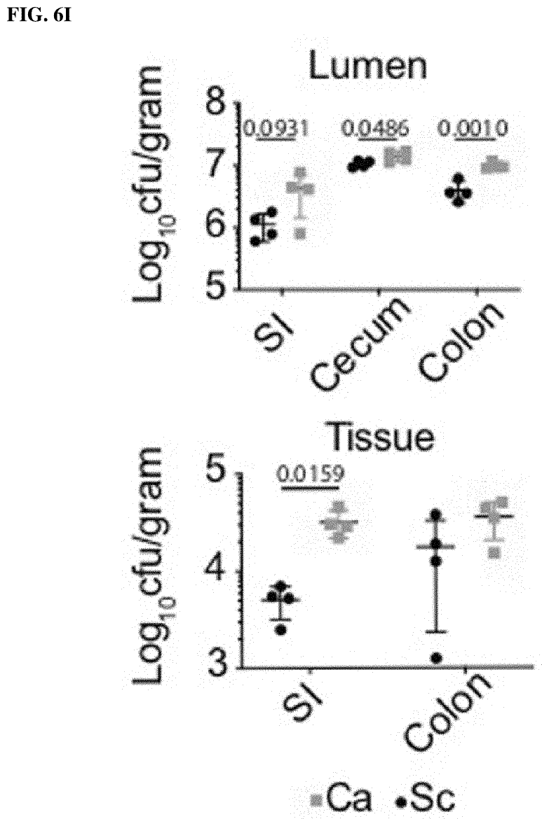

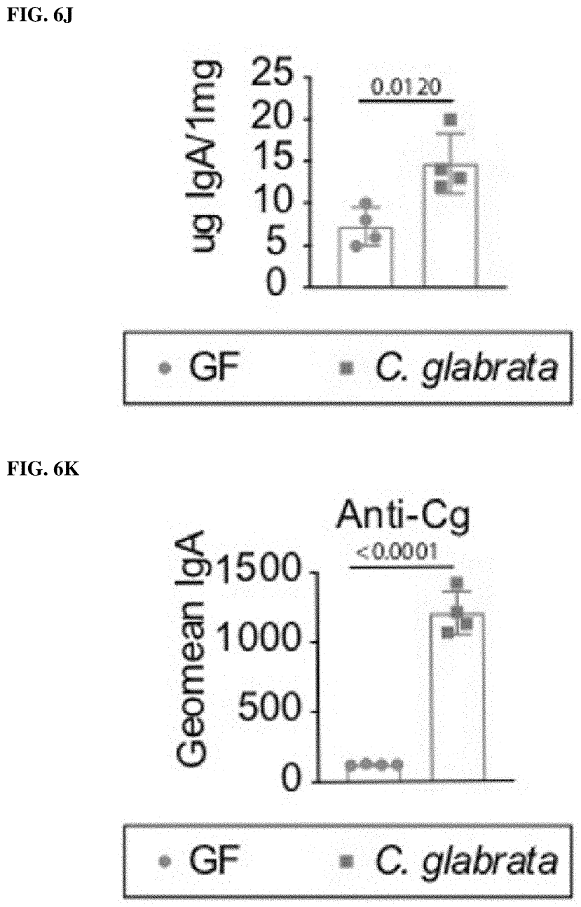

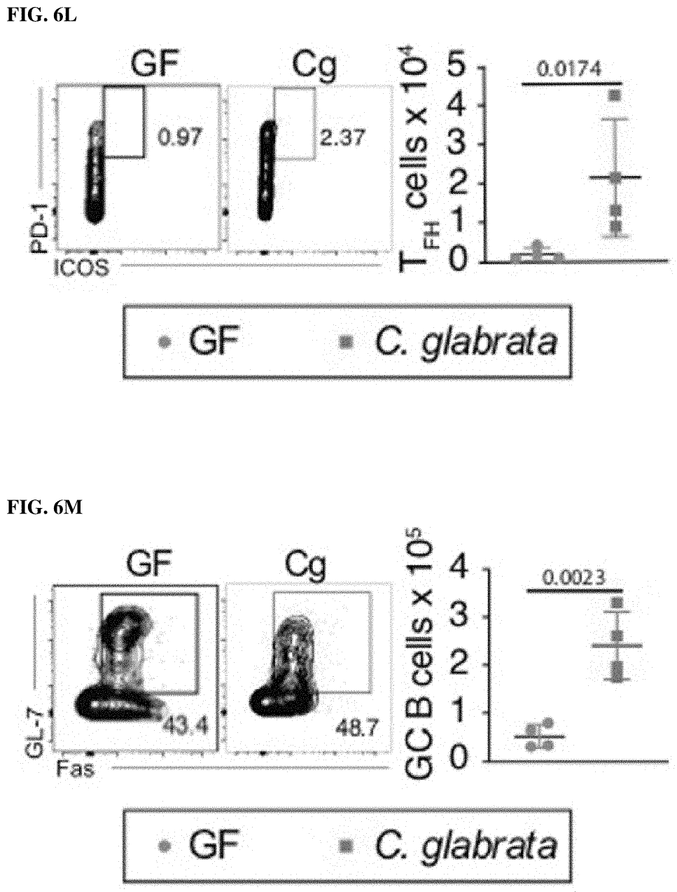

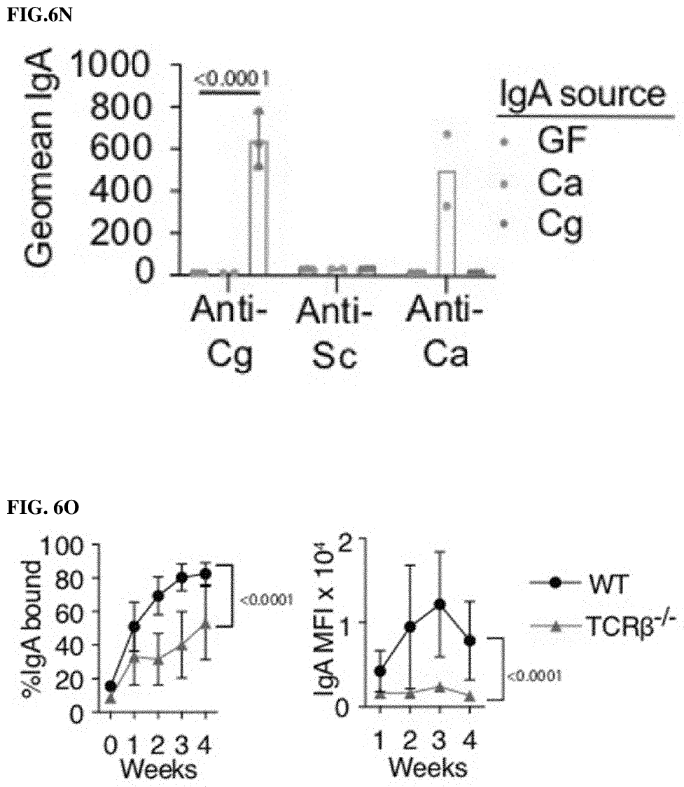

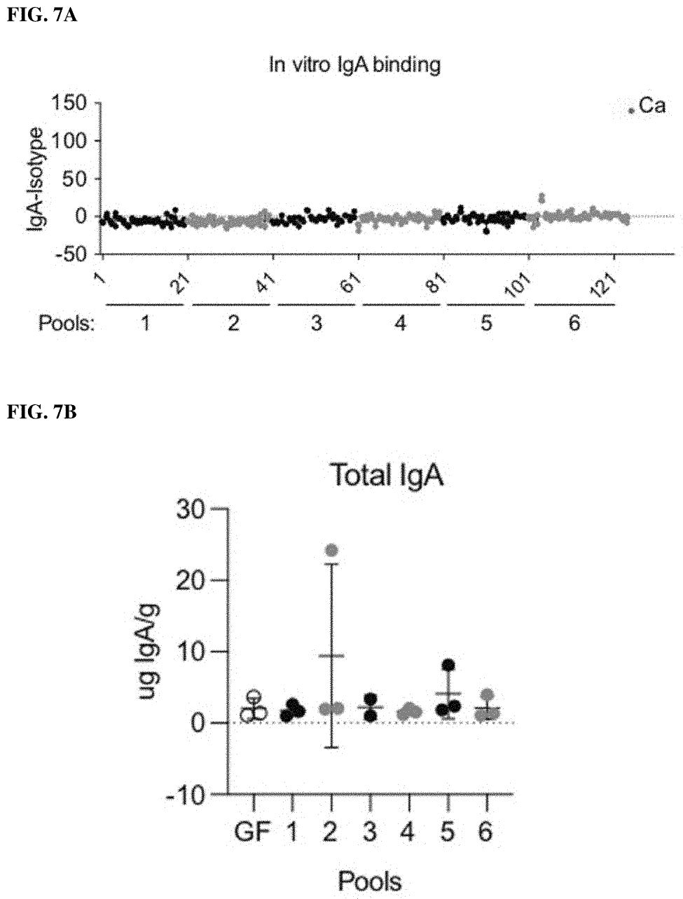

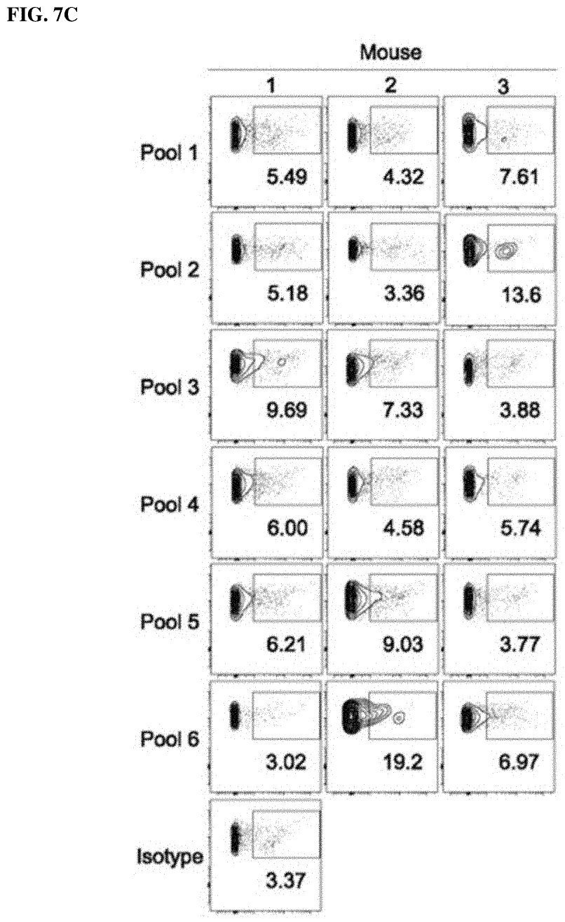

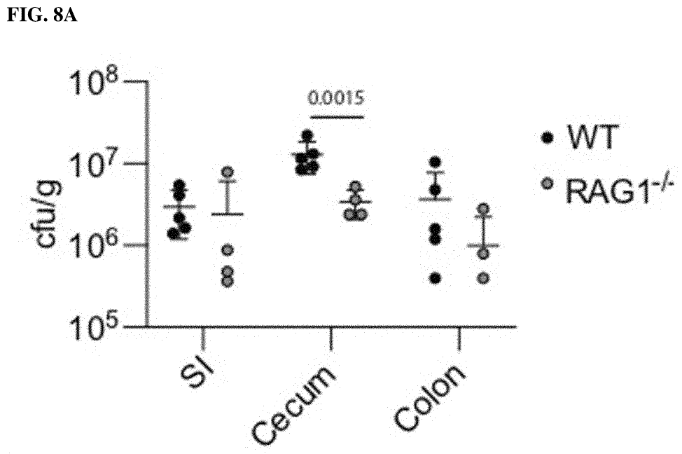

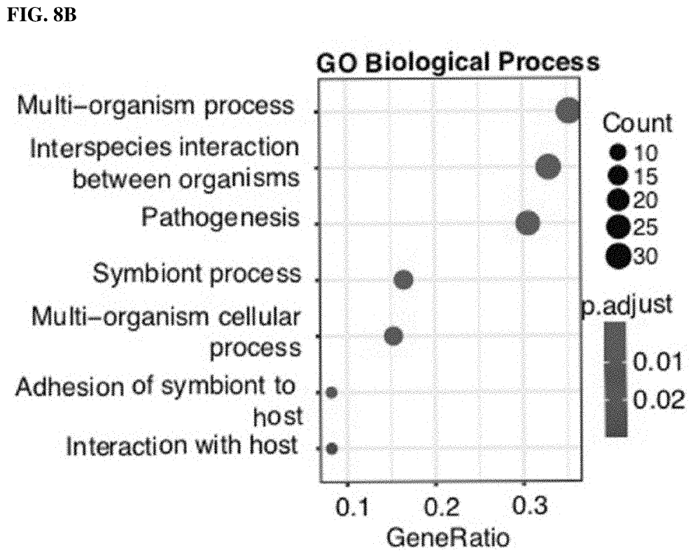

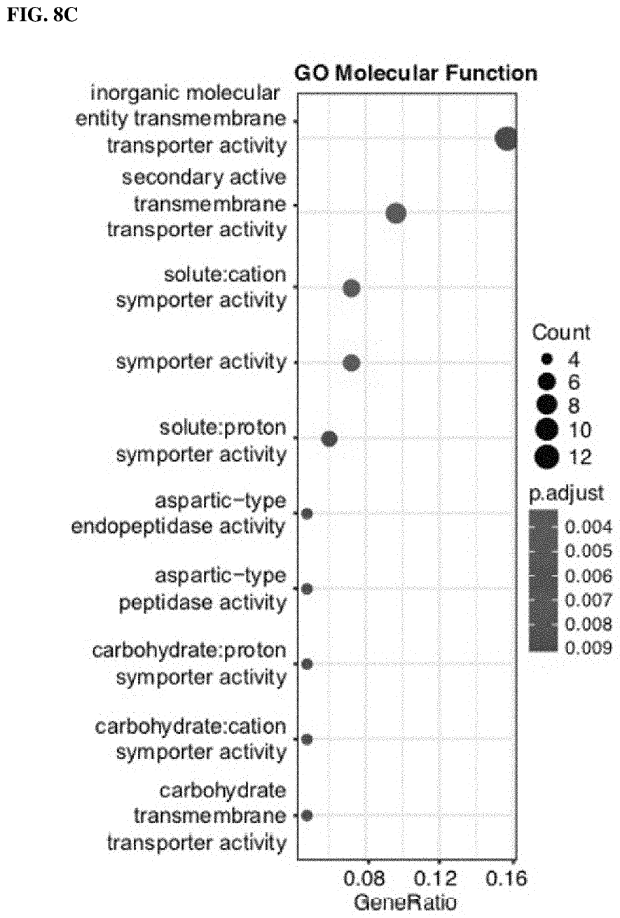

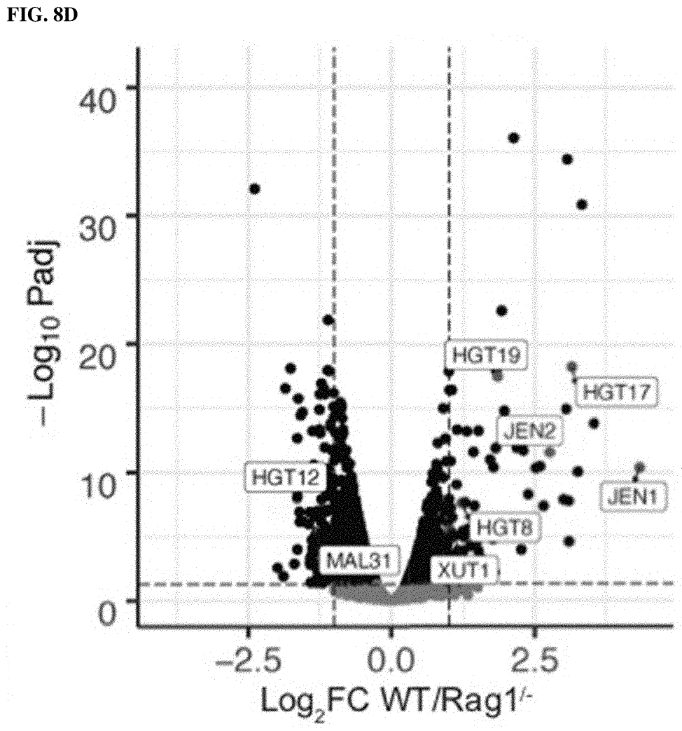

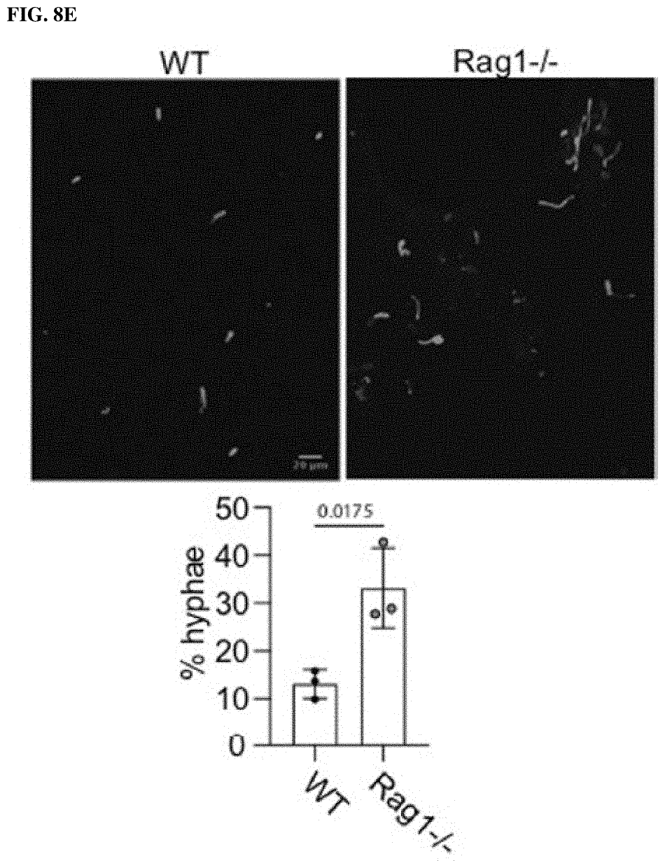

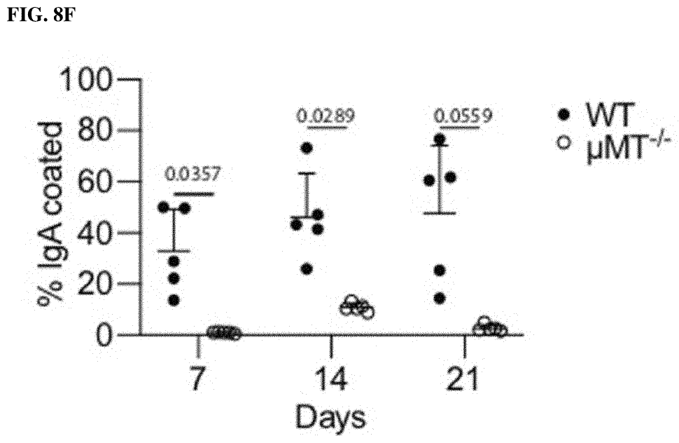

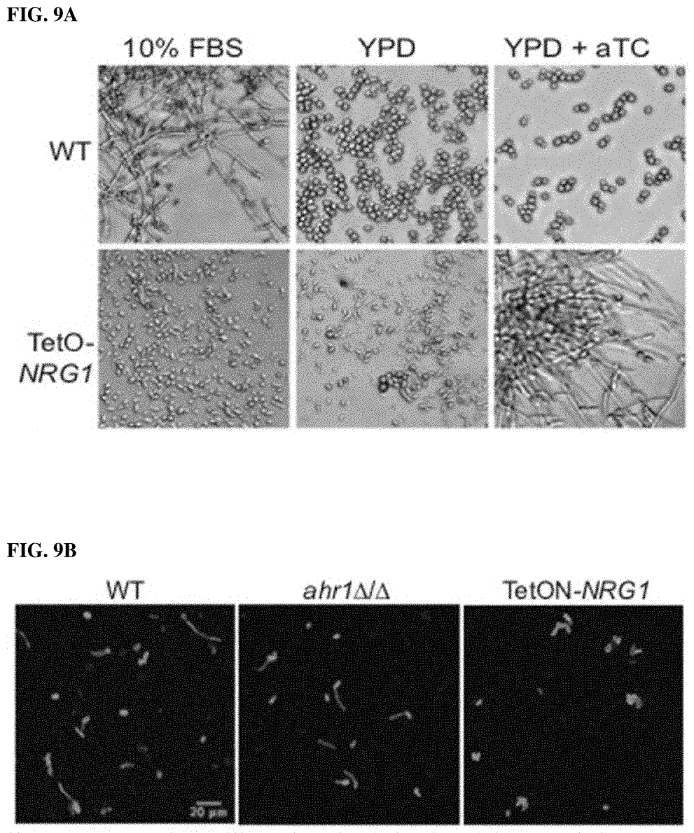

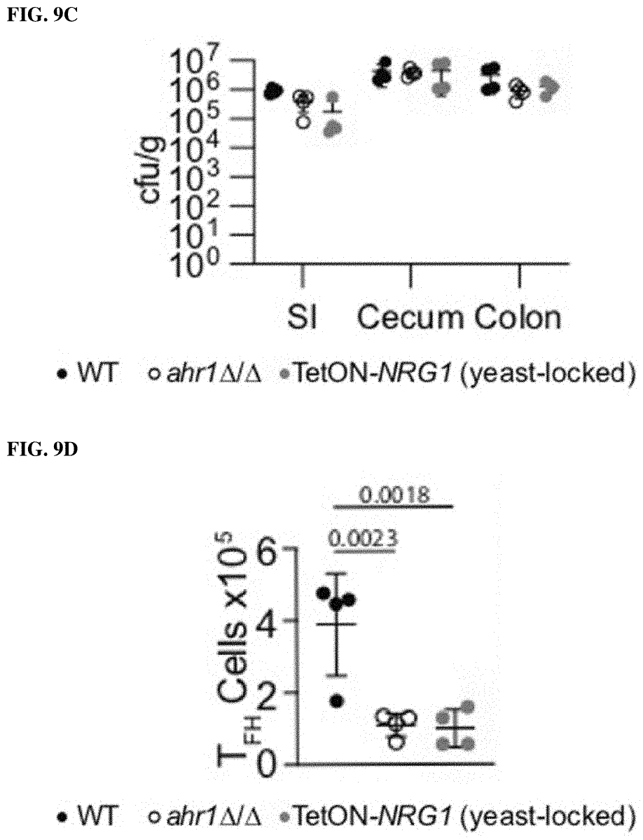

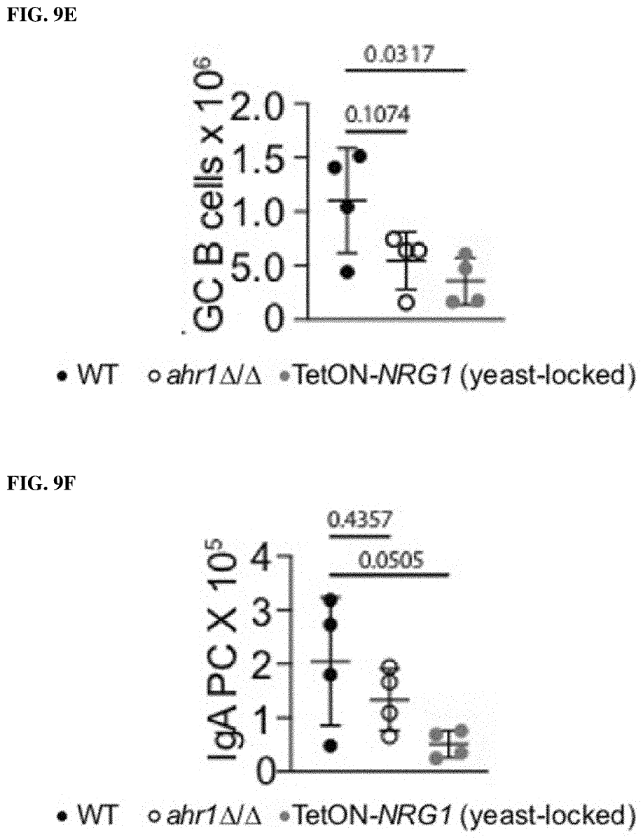

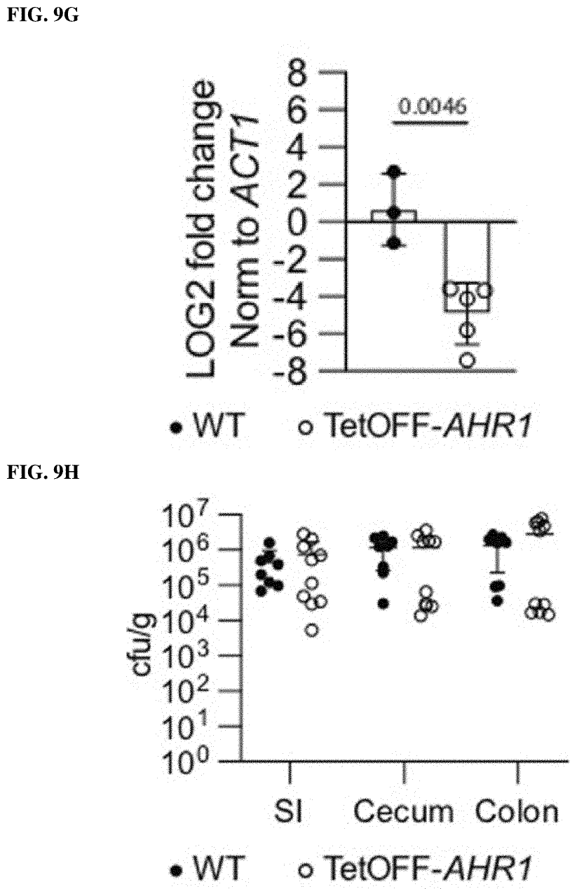

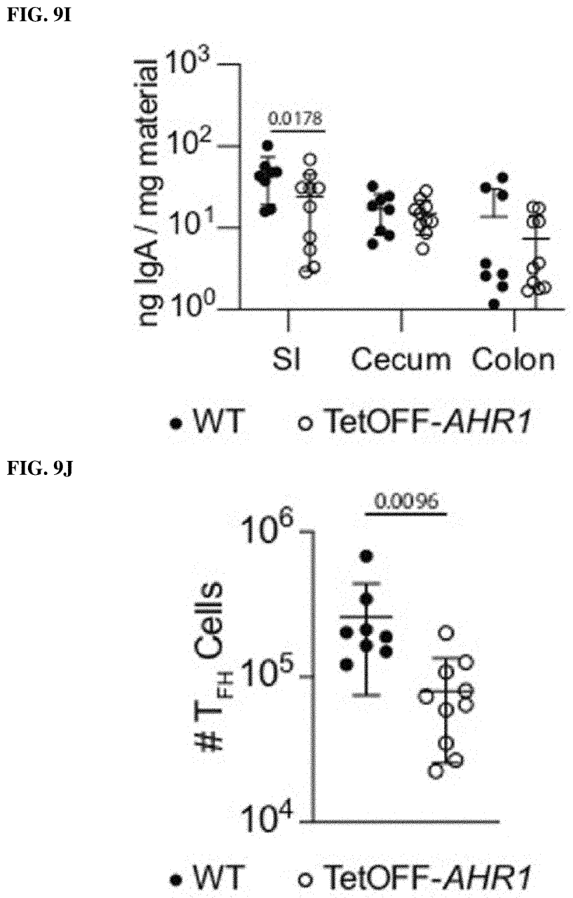

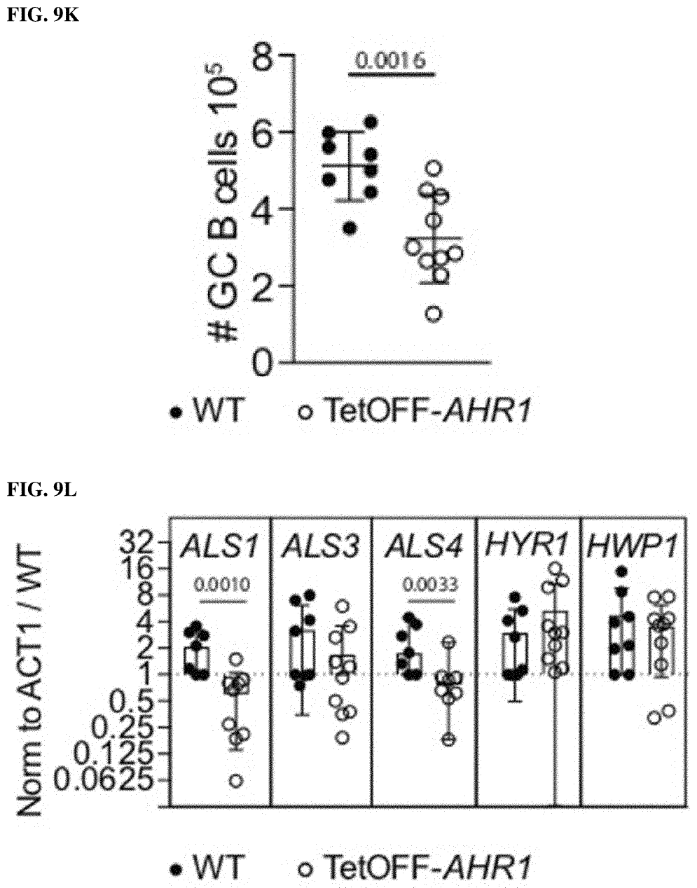

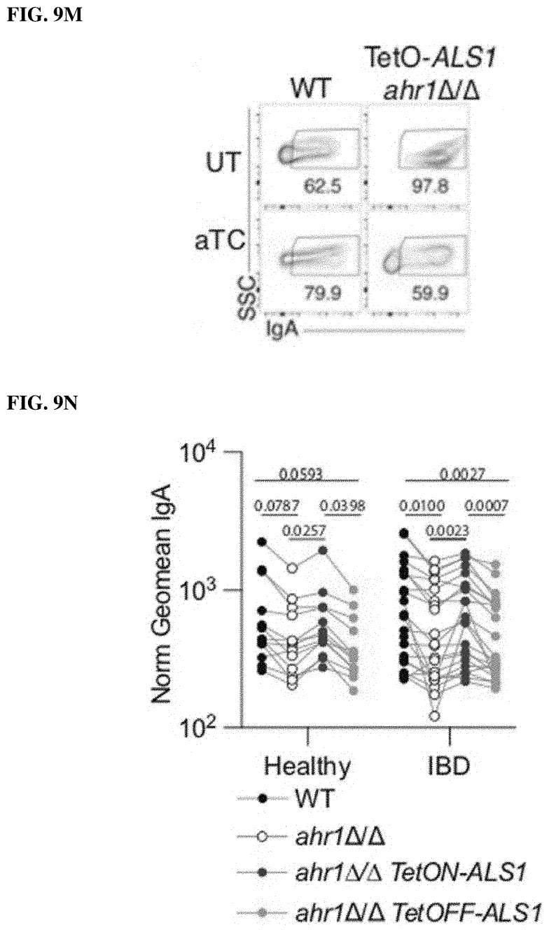

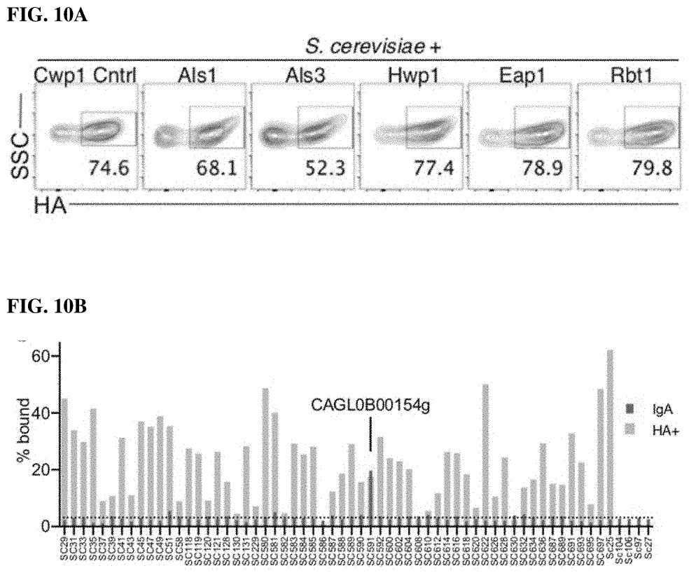

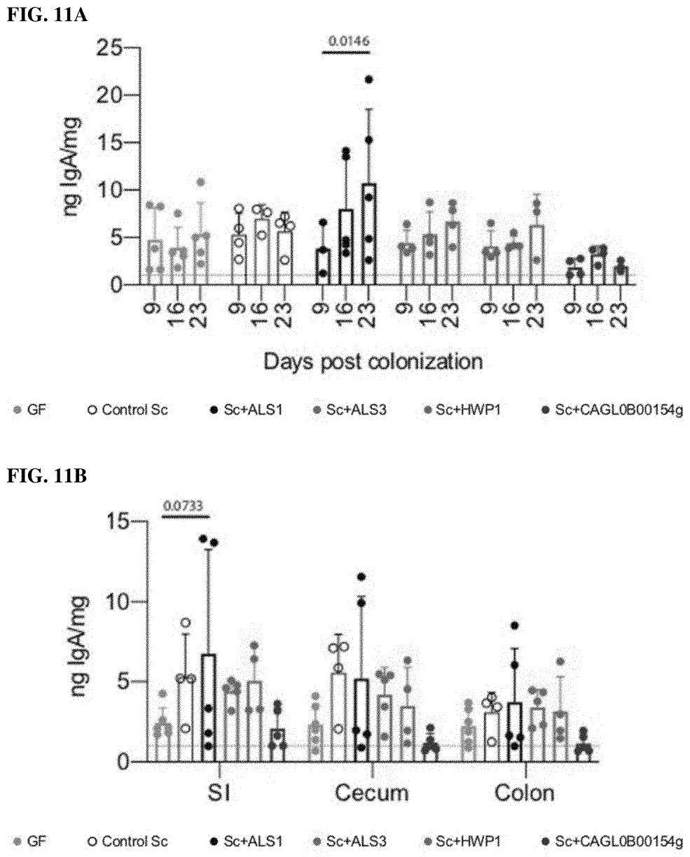

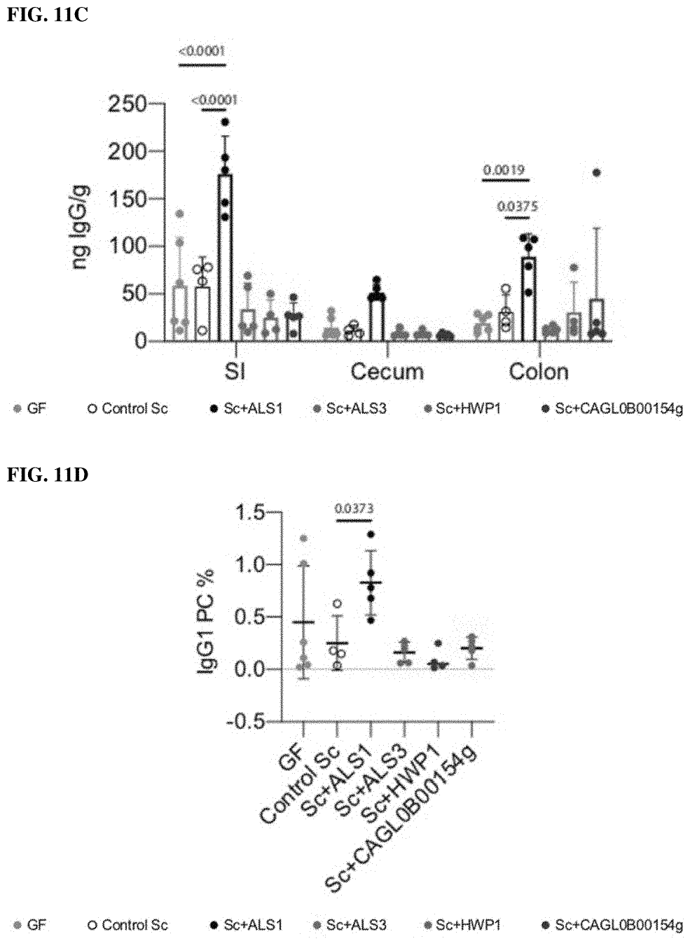

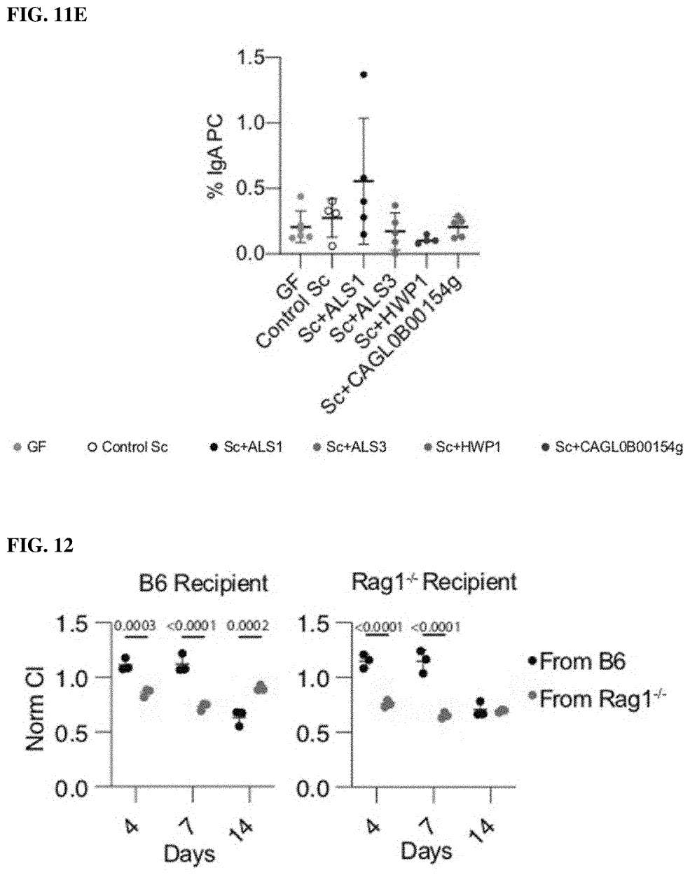

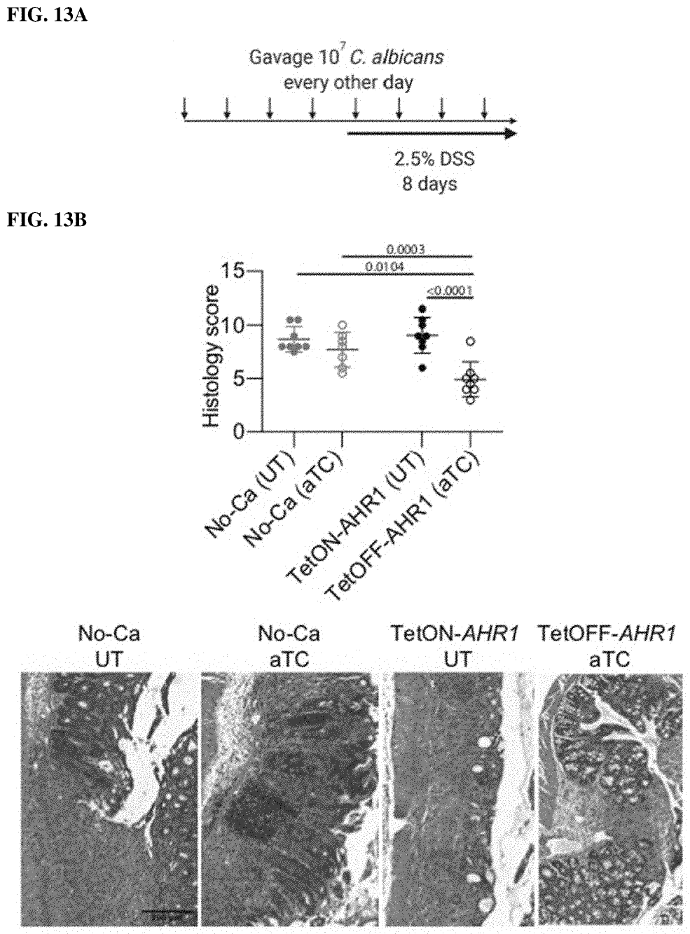

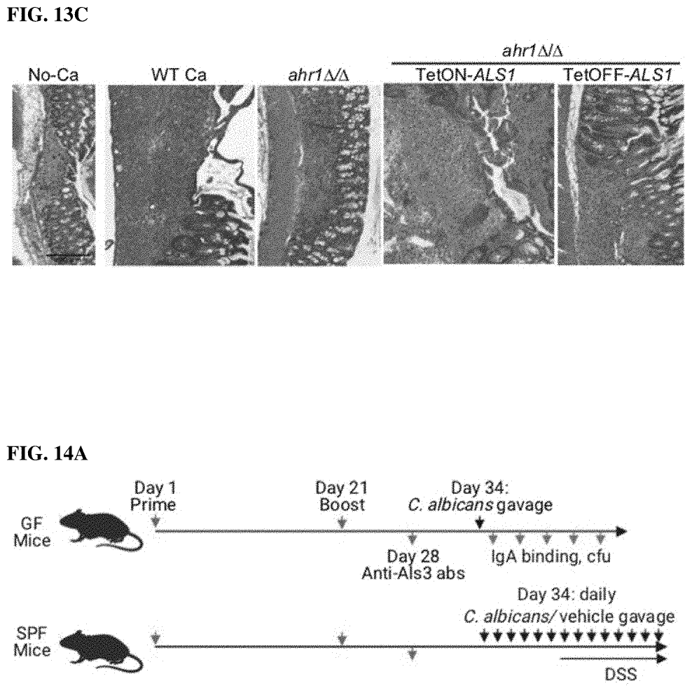

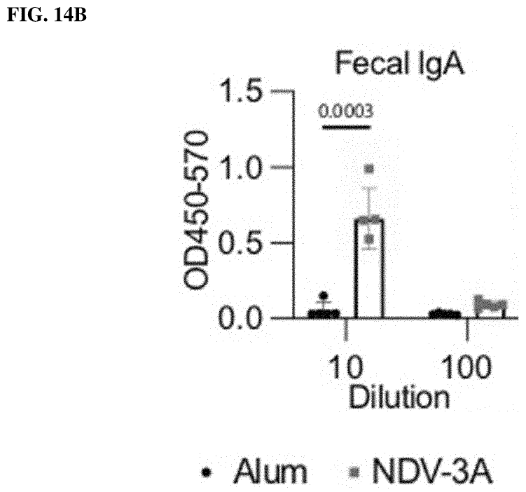

FIG. 1J shows faecal C. albicans in antibiotic-treated wild-type and μMT^{-/-} mice three weeks after inoculation (n=5 mice per group; one experiment). Scale bars for both 1I and 1J are 50 μm.

FIG. 1K shows imaging flow cytometry images of IgA⁺ C. albicans from monocolonized mice. IgA⁺ and IgA⁻ populations assessed via object circularity score. Brightfield (BF), calcofluor white (CFW), IgA, and CFW and IgA composite. Data are pooled from three B6 mice monocolonized with C. albicans three weeks after inoculation and are representative of two experiments.

FIGS. 2A-2G show that IgA targets C. albicans adhesins. P values calculated using one-way ANOVA with Tukey's test (FIGS. 2C, 2D and 2G), or Holm-Sidak's test (FIG. 2F). Data are mean±s.d. (FIGS. 2C, 2D, 2F and 2G).

FIG. 2A shows strains from the Noble (white bars) and Homann (grey bars) collections with an IgA binding z-score of ≤−2. Intestinal wash from SW and B6 mice that were monocolonized with C. albicans. Strains in bold were identified in both collections (n=2; one experiment). The Noble collections were described in Noble, S. M., French, S., Kohn, L. A., Chen, V. & Johnson, A. D., “Systematic screens of a Candida albicans homozygous deletion library decouple morphogenetic switching and pathogenicity,” Nat. Genet. 42, 590-598 (2010), and the Homann collections were described in Homann, O. R., Dea, J., Noble, S. M. & Johnson, A. D., “A phenotypic profile of the Candida albicans regulatory network,” PLoS Genet. 5, e1000783 (2009), both of which are incorporated by reference herein in their entirety.

FIG. 2B shows Gene Ontology (GO) biological process terms that were enriched in the strains from FIG. 2A.

FIG. 2C shows small intestinal IgA. Normalized to PMA1 (n=4 mice per group).

FIG. 2D shows ALS1 quantitative PCR with reverse transcription (qRT-PCR) from the caecum contents of monocolonized mice. Data was normalized to PMA1 (n=4 mice per group).

FIG. 2E shows cell-wall fractions probed with intestinal-wash IgA from C. albicans-monocolonized mice. Als3 was identified only in the hyphal fraction using LC-MS/MS (representative of two experiments).

FIG. 2F shows IgA reactivity of monocolonized B6 (circles) or SW (squares) mice to the control S. cerevisiae, which expresses the CWP1 cell-surface scaffold but not the adhesin, or S. cerevisiae expressing the indicated C. albicans adhesins. Binding intensity was normalized to strains stained without faecal wash (FW) (n=7 B6 and n=8 SW mice).

FIG. 2G shows human faecal IgA reactivity to S. cerevisiae strains expressing the indicated C. albicans adhesins, Cg-Ad, CAGL0B00154g. Staining intensity for each human sample was normalized to the control S. cerevisiae strain expressing no adhesins (n=70).

FIGS. 3A-3D show that adaptive immunity enhances the competitive fitness of C. albicans. P values calculated using two-way ANOVA with Tukey's test (FIG. 3B), one-way ANOVA with Tukey's test of area under the curve (FIG. 3D) or two-sided unpaired t-test (3FIG. C). Data are mean±s.d. (FIGS. 3B-3D).

FIG. 3A shows schematic of intestinal conditioning of C. albicans in wild-type and Rag1^{-/-} GF mice and subsequent competition in naive mice. Silhouettes in FIG. 3A were created using BioRender.

FIG. 3B shows competitive index (CI) four days after inoculating antibiotic-treated mice with conditioned C. albicans (n=2 B6 and n=3 Rag1^{-/-} mice (2 days); n=3 mice per group (4 weeks)).

FIG. 3C shows competitive index (CI) seven days after inoculating GF B6 mice with conditioned wild-type or TetOn-NRG1 (yeast-locked), C. albicans (n=3 WT-colonized and n=4 TetOn-NRG1-colonized mice). The competitive index was normalized to that when strains were competed in wild-type and Rag1^{-/-} mice directly from culture.

FIG. 3D shows competitive index of wild-type versus TetOn-ALS3 or TetOff-ALS3 C. albicans during colonization of antibiotic-treated B6 mice that were untreated (UT) or treated with aTC (n=4 mice per group).

FIGS. 4A-4G show that vaccination against C. albicans adhesins prevents damage during colitis. P values calculated using two-way ANOVA with Sidak's test (FIG. 4D), two-sided unpaired t-test (FIGS. 4E and 4F) or one-way ANOVA with Tukey's test (FIGS. 4B, 4C and 4G). Data are mean±s.d. (FIGS. 4B-4G).

FIG. 4A and FIG. 4B show Dextran Sulfate Sodium (DSS) histology images (4A) and DSS histology scores (4B) for mice treated with no C. albicans (No Ca), wild-type C. albicans (WT), TetOn-NRG1 C. albicans (yeast-locked) or TetOff-NRG1 (aTC-treated) C. albicans (hyphal-locked) (n=5 mice per group). Scale bar is 200 μm for both figures.

FIG. 4C shows DSS histology scores for mice treated with no C. albicans or with wild-type C. albicans, ahr1Δ/Δ C. albicans, TetOn-ALS1 ahr1Δ/Δ C. albicans or TetOff-ALS1 ahr1Δ/Δ (aTC-treated) C. albicans (n=10 (no Ca, WT and ahr1Δ/Δ), n=5 (TetOn-ALS1) and n=4 (TetOff-ALS1) mice per group).

FIG. 4D shows IgA binding to faecal C. albicans in monocolonized alum or NDV-3A-vaccinated mice quantified by flow cytometry.

FIG. 4E shows colon-tissue-associated colony-forming units (CFU).

FIG. 4F shows ALS3 and ALS1 expression quantified by qRT-PCR from the colon contents of C. albicans-monocolonized mice treated as in FIG. 4D (n=5 mice per group).

FIG. 4G shows DSS histology scores for alum- or NDV-3A-vaccinated mice treated with no C. albicans or wild-type C. albicans (n=10 mice per group).

FIGS. 5A-5E show human faecal and serum anti-fungal antibodies. P values calculated using two-way ANOVA with Tukey's test (FIG. 5A), one-way ANOVA with Dunn's test (FIG. 5C) or two-sided Mann-Whitney U-test (FIGS. 5F and 5D).

FIG. 5A shows human faecal antibody binding to cultured fungi and quantified by flow cytometry after staining with fluorescent secondary antibodies (n=70). Staining intensity normalized to fungi stained with secondary antibodies but without human faecal wash. Box plots show minimum 25% quartile and maximum 75% quartile around the median and whiskers show range.

FIG. 5B shows IgA binding to cultured fungi from serial dilutions of human faecal wash (n=30 healthy, n=23 Crohn's disease, and n=17 UC). Geometric mean was 95% confidence intervals (CI).

FIGS. 5C and 5D show human serum antibody binding to cultured fungi. Serum diluted 1:75. (n=12, n=4 healthy, and n=8 Crohn's disease).

FIG. 5E shows faecal ASCA (anti-Saccharomyces cerevisiae antibody) IgA levels from undiluted faecal wash (n=30 healthy, n=18 Crohn's disease and n=14 UC). Median with 95% confidence intervals (CI).

FIG. 5F shows serum ASCA (anti-Saccharomyces cerevisae antibody) IgA levels in healthy patients (“Healthy”, n=4) and in patients with Crohn's disease (“CD”, n=8).

FIGS. 6A-6O show that IgA targets Candida species but not S. cerevisiae. P values calculated using two-way ANOVA (FIGS. 6D and 6O), with Sidak's test (FIGS. 6B, 6E, 6F, 6G, 6H and 6N), or two-sided unpaired t-test (FIGS. 6J, 6K, 6L and 6M). Mean values±s.d. for FIGS. 6B and 6D-6O.

FIG. 6A shows IgA-bound faecal fungi gating strategy.

FIG. 6B shows Peyer's patch GC B cell and TFH cell gating strategy.

FIG. 6C shows colon LP IgA plasma cell gating strategy (n=4 mice per group 30 days after inoculation).

FIG. 6D shows IgA binding to faecal GFP⁺ S. cerevisiae and GFP⁺ C. albicans in monocolonized SW mice.

FIG. 6E shows the total IgA levels from monocolonized SW mice.

FIG. 6F shows flow cytometry quantification of SW IgA binding to cultured S. cerevisiae and C. albicans (n=4 C. albicans-colonized and n=5 S. cerevisiae-colonized).

FIGS. 6G and 6H show serum antibody binding to cultured C. albicans or S. cerevisiae from SW (FIG. 6G) or B6 (FIG. 6H) GF or monocolonized mice. Antibody quantified by flow cytometry from serum diluted 1:25 (SW: GF n=4, Sc-colonized n=5, Ca-colonized n=5; B6: GF n=3, Sc n=5, Ca-colonized n=3).

FIG. 6I shows lumen and tissue-associated fungal burden in monocolonized B6 mice 30 days after inoculation (n=4 mice per group).

FIG. 6J shows whole-intestinal IgA four weeks after inoculation.

FIG. 6K shows caecal wash IgA binding to cultured C. glabrata measured by flow cytometry.

FIG. 6L shows Peyer's patch TFH cells four weeks after inoculation.

FIG. 6M shows Peyer's patch GC B cells (n=4 mice per group).

FIG. 6N shows IgA binding to cultured C. glabrata, S. cerevisiae and C. albicans from faecal wash from GF, C. albicans-monocolonized or C. glabrata-monocolonized intestinal wash (n=2 C. albicans, n=3 C. glabrata and n=3 S. cerevisiae faecal washes)

FIG. 6O shows percentage of IgA binding and binding intensity of faecal C. albicans during colonization of antibiotic-treated wild-type and TCRβ^{-/-} mice (n=6 TCRβ^{-/-} and n=8 wild-type mice from two experiments).

FIGS. 7A-7C show that an IgA response is not induced by 124 distinct S. cerevisiae strains.

FIG. 7A shows IgA binding to the 20-24 strains from each pool was assessed by flow cytometry. Mice were gavaged weekly with the indicated pool for three weeks and caecal wash from mice was used as a source of IgA. C. albicans bound by IgA from C. albicans-monocolonized mice is shown in red.

FIG. 7B shows the total IgA in caecum contents quantified by ELISA. Mean values±s.d.

FIG. 7C shows IgA binding to S. cerevisiae (pre-gated on CFW intermediate) populations from caecal material. (n=3 mice per group representative of two experiments).

FIGS. 8A-8F show fungal burden and Gene Ontology (GO) term enrichment analysis of RNA-seq comparison of C. albicans in monocolonized wild-type and Rag1^{-/-} mice. P values calculated using two-way ANOVA with Sidak's multiple comparisons test (FIGS. 8A and 8F) or two-sided unpaired t-test (FIG. 8E).

FIG. 8A shows fungal burden in wild-type and Rag1^{-/-} mice monocolonized with C. albicans four weeks after inoculation. Mean values±s.d.

FIG. 8B and FIG. 8C show biological process (FIG. 8B) or molecular function (FIG. 8C) GO term enrichment in genes with q≤0.05 and log 2-transformed fold change ≥1 or ≤−1.

FIG. 8D shows volcano plot of the ratio of C. albicans transcripts in wild-type and Rag1^{-/-} mice with active transmembrane transporter activity genes labelled in red (n=5 wild type and 4 Rag1^{-/-} mice for FIGS. 8A-8D).

FIG. 8E shows C. albicans morphology in colon contents from monocolonized wild-type or Rag1^{-/-} mice four weeks after colonization. Mean values±s.d. (n=3 mice per group).

FIG. 8F shows IgA binding to C. albicans in the faeces of antibiotic-treated wild-type and μMT^{-/-} mice four weeks after inoculation. Mean values±s.d. (n=5 mice per group).

FIGS. 9A-9N show that filamentation and Ahr1 promote intestinal IgA responses. P values calculated using one-way ANOVA with Tukey's test (FIGS. 9C-9F), two-way ANOVA with Sidak's test (9I), two-sided unpaired t-test (FIGS. 9J, 9K and 9L), two-sided Mann-Whitney U-test (FIG. 9G) or Friedman test with Dunn's test (FIG. 9N).

FIG. 9A shows morphology of indicated C. albicans strains incubated for 4 hr. in RPMI with 10% FBS, YPD, or YPD+5 μg/ml aTC). TetO-NRG1 constitutively expresses NRG1 when untreated (TetON), but aTC repressed NRG1 expression (TetOFF).

FIG. 9B shows C. albicans in caecum contents stained with AF488 anti-Candida antibody.

FIG. 9C shows intestinal fungal burden (mean values±s.d.).

FIG. 9D shows Peyer's patch T_{FH} cells (ICOS⁺PD-1⁺CD4⁺CD3⁺ live cells) (mean values±s.d.).

FIG. 9E shows Peyer's patch GC B cells (GL-7⁺Fas⁺IgD-CD19⁺ live cells) (mean values±s.d.).

FIG. 9F shows colon LP IgA⁺ plasma cells (IgA⁺CD138⁺CD45⁺CD3⁻CD19⁻ live cells) (mean values±s.d.) quantified from mice monocolonized for four weeks (for 9C-9F, n=4 mice per group).

FIG. 9G shows faecal AHR1 qPCR in aTC-treated mice monocolonized with wild-type or TetO-AHR1 (TetOff-AHR1) (wild type n=3 and TetOff-ALS1 n=5). Mean values±s.d.

FIG. 9H shows fungal burden of wild-type- and TetOff-AHR1-monocolonized mice.

FIG. 9I shows IgA from wild-type- or TetOff-AHR1-monocolonized mice.

FIGS. 9J and 9K show Peyer's patch TFH cells (FIG. 9J) and Peyer's patch GC B cells (FIG. 9K) from mice monocolonized with wild type or TetOff-AHR1.

FIG. 9L shows qRT-PCR from the small intestinal contents of monocolonized mice (for FIGS. 9H-9L, wild type n=8 and TetOff-ALS1 n=10 mice per group from two experiments).

FIG. 9M shows Intestinal IgA (from C. albicans-monocolonized mice) binding to strains that were cultured untreated or were treated with aTC.

FIG. 9N shows human IgA binding to indicated strains cultured without aTC (wild type, ahr1 Δ/Δ, ahr14/4 TetOn-ALS1) or with 5 μg ml-1 aTC (ahr1 Δ/Δ TetOff-ALS1). IgA binding quantified by flow cytometry (healthy n=13 and IBD n=22. Samples chosen had enough C. albicans-reactive IgA to bind at least 10% of cultured wild-type C. albicans).

FIG. 10A-10B show that C. albicans and C. glabrata-induced IgA targets adhesins or adhesin-like proteins.

FIG. 10A shows anti-HA (hemagglutinin) staining of the control S. cerevisiae expressing the Cwp1 scaffold and the S. cerevisiae strains expressing HA-tagged C. albicans adhesins.

FIG. 10B shows anti-HA and IgA binding to S. cerevisiae strains expressing HA-tagged C. glabrata adhesins after incubation in cecal wash from mice monocolonized with C. glabrata. SC104, SC106, SC97, and SC27 express adhesins not tagged by HA. HA and IgA binding quantified by flow cytometry.

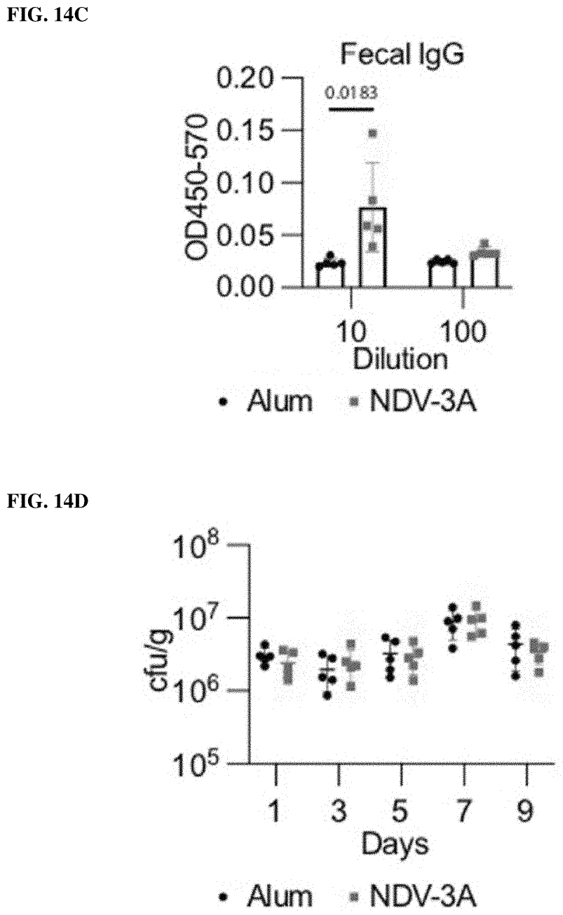

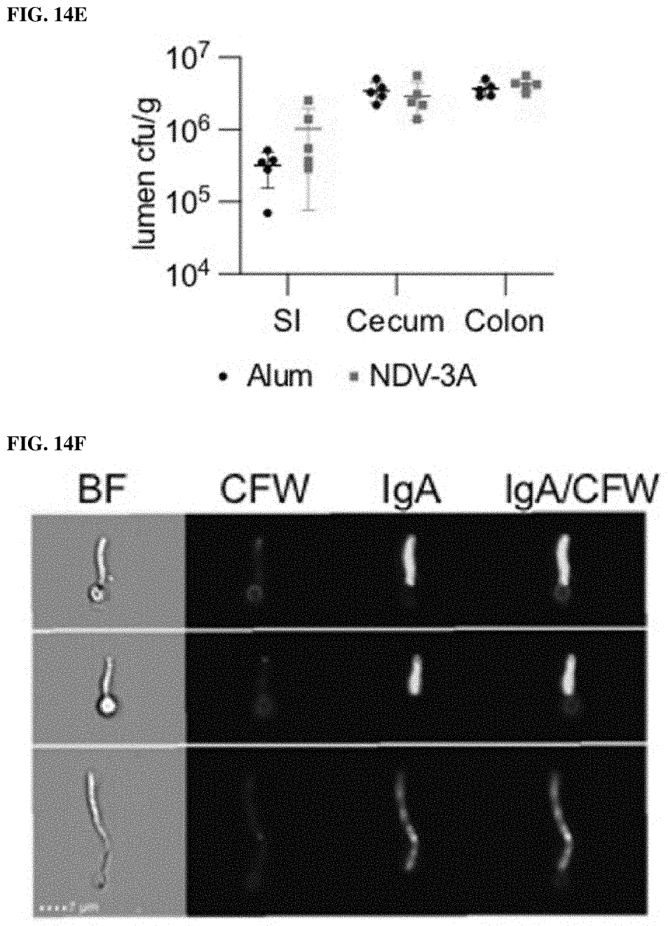

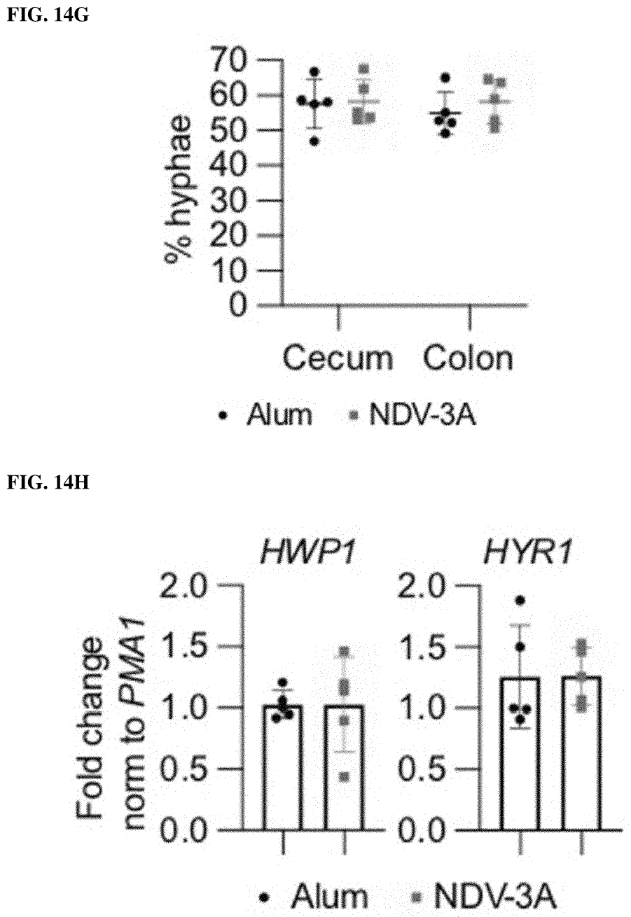

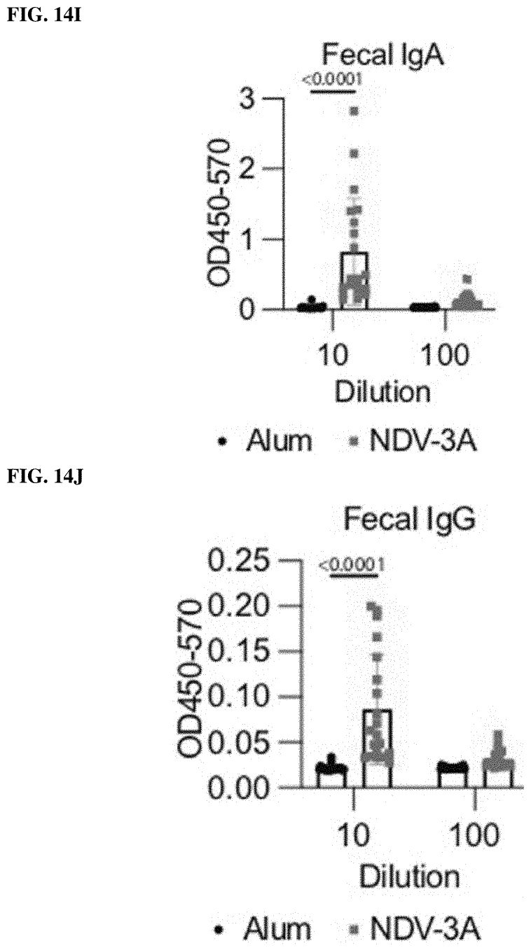

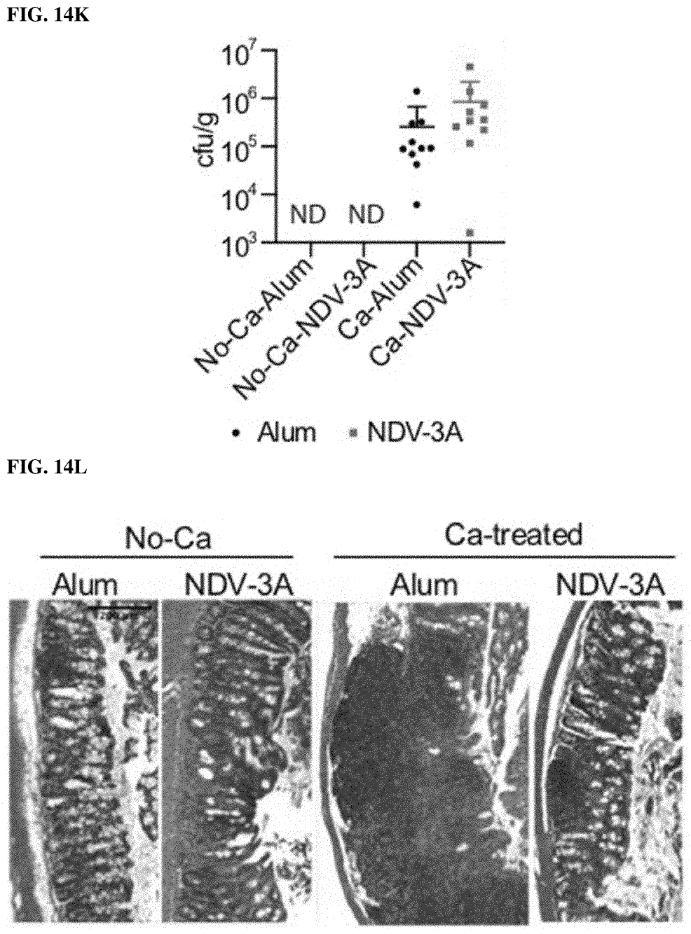

FIGS. 11A-11E show that antibody induction by S. cerevisiae strains expressing Candida adhesins. GF SW mice were monocolonized with the indicated strains or left GF. Colonized mice were gavaged three times per week with cultured strains. The control S. cerevisiae expresses the CWP1 cell surface scaffold but not an adhesin. P values calculated using one-way ANOVA with Tukey's test (FIGS. 11D, 11E) or two-way ANOVA with Tukey's test (FIGS. 11A-11C). All data are mean±s.d.

FIG. 11A shows weekly faecal IgA levels normalized by faecal weight.

FIGS. 11B and 11C show intestinal IgA (FIG. 11B) and IgG (FIG. 11C) levels at day 28 normalized by material weight.

FIG. 11D shows colon lamina propria IgG1 plasma cells (live IgG1⁺IgA⁻CD138⁺CD19⁻CD3⁻CD45⁺ live cells).

FIG. 11E shows colon lamina propria IgA plasma cells (live IgA⁺IgG1⁻CD138⁺CD19⁻CD3⁻CD45⁺ live cells) (for FIGS. 11A-11E, GF n=6, control Sc n=4, Sc+Als1 n=5, Sc+Als3 n=5, Sc+Hwp1 n=4, Sc+CAGL0B00154g n=5 mice per group).

FIG. 12 shows immune-enhanced fitness diminishes after 14 days. Competitive index (CI) of C. albicans conditioned for four weeks in indicated GF recipient mice. B6-conditioned C. albicans was iRFP⁺ and Rag1^{-/-} conditioned C. albicans was Neon⁺. CI normalized to the CI when strains were competed in wild-type and Rag1^{-/-} mice directly from culture (competition mice, n=3 B6 and n=4 Rag1^{-/-} mice from one experiment). P values calculated using two-way ANOVA with Sidak's test. Data are mean±s.d.

FIGS. 13A-13C show that AHR1 exacerbated DSS colitis. P values calculated using two-way ANOVA with Tukey's test (FIG. 13B).

FIG. 13A shows schematic of DSS colitis experiments.

FIG. 13B shows histology images and scores for mice treated with no C. albicans or with TetO-AHR1 with and without aTC (no-Ca UT, no-Ca aTC and TetOn-AHR1 UT n=7 mice per group, TetOff-AHR1 aTC n=8 mice per group from two independent experiments). Data are mean±s.d.

FIG. 13C shows DSS histology images for mice treated with no C. albicans or with wild-type C. albicans, ahr1Δ/Δ C. albicans, TetOn-ALS1 ahr1Δ/Δ C. albicans and TetOff-ALS1 ahr1Δ/Δ C. albicans (aTC-treated).

FIGS. 14A-14L show that NDV-3A induces an intestinal anti-Als3 antibody response. P values calculated using two-way ANOVA with Sidak's test (FIGS. 14B, 14C, 14I and 14J). All data are mean±s.d. Silhouettes in a were created using BioRender.

FIG. 14A shows model of monocolonization and DSS experiment in vaccinated mice.

FIGS. 14B and 14C show ELISA quantification of Als3-specific IgA (FIG. 14B) and IgG (FIG. 14C) from the faeces of GF mice one week after boost with alum of NDV-3A vaccine.

FIGS. 14D and 14E show faecal (FIG. 14D) and intestinal (FIG. 14E) lumen CFU of C. albicans in monocolonized alum or NDV-3A vaccinated mice. Intestinal CFU quantified 12 days after colonization.

FIG. 14F shows imaging flow cytometry images of IgA⁺ C. albicans from caecum of NDV-3A vaccinated mice.

FIG. 14G shows the percentage of hyphae quantified using an AF488 anti-Candida antibody to visualize morphology from indicated intestinal region 12 days after monocolonization.

FIG. 14H shows the HWP1 and HYR1 transcripts quantified by qRT-PCR from colon C. albicans 12 days after monocolonization. (for FIGS. 14B-14H, n=5 mice per group).

FIGS. 141 and 14J show that ELISA quantification of Als3-specific IgA (FIG. 14I) and IgG (FIG. 14J) in the faeces of conventionally colonized mice used for the DSS experiment.

FIG. 14K shows C. albicans CFU in colon contents after DSS treatment (for FIGS. 14I-14K, n=10 mice per group).

FIG. 14L shows example H&E-stained histology images from the NDV-3A DSS experiment.

### DETAILED DESCRIPTION OF THE INVENTION

The compositions and methods disclosed herein are based, at least in part, on the identification and characterization of pathogenic hyphal morphotype, which is specialized for adhesion and invasion, and preferentially targeted and suppressed by intestinal IgA responses.

In this disclosure, “comprises,” “comprising,” “containing” and “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Subjects

The term “subject” refers to animals, typically mammalian animals. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. A mammal can be a pregnant female. In certain embodiments a mammal can be an animal disease model, for example, animal models used for the study of an intestinal disease. In some embodiments, the intestinal disease is inflammatory bowel disease (IBD), such as Crohn's disease or colitis. In some embodiments, the intestinal disease is caused by, or exacerbated by, fungi in the Candida genus. In some embodiments, the intestinal disease is caused by, or exacerbated by, an infection by fungi in the Candida genus. In some embodiments, the intestinal disease is caused by, or exacerbated by, the propagation of Candida species forming adhesive hyphae.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

In some embodiments, a subject in need of a treatment or composition described herein is a subject at risk of intestinal disease and/or a subject that has an intestinal disease. In some embodiments, a subject in need of a treatment or composition described herein is a subject at risk of inflammatory bowel disease and/or a subject that has an inflammatory disease. In some embodiments, a subject in need of a treatment or composition described herein is infected with, or is suspected of being infected with a Candida pathogen. In certain embodiments an antibody binding agent (e.g., an antibody or the like), a polypeptide, or composition described herein is used to treat or prevent a Candida infection or Candida propagation in a subject or a subject at risk of acquiring an intestinal disease.

### Pharmaceutical Compositions

In some embodiments, a composition comprises one or more Candida adhesin polypeptides (e.g., Als1, Als3, HYR1, or HWP1), or portions thereof, and one or more adjuvants. In certain embodiments, a composition is an immunogenic composition. In some embodiments, provided herein is a composition comprising one or more toxin polypeptides, or portions thereof, and one or more adjuvants for use as a vaccine.

In some embodiments, a composition comprises one or more polypeptides comprising 5 to 500, 5 to 400, 5 to 300, 5 to 200 or 5 to 100 consecutive amino acids selected from the wild-type sequences of Als1, Als3, HYR1, or HWP1, and an adjuvant. In some embodiments, a composition comprises one or more polypeptides comprising 5 or more, 10 or more, 15 or more, 16, or more, 17 or more, 18 or more, 19 or more, 20 or more, 25 or more or 30 or more consecutive amino acids selected from wild-type sequences of Als1, Als3, HYR1, or HWP1, and an adjuvant. In some embodiments, a composition comprises one or more polypeptides each comprising 10, 11, 12, 13, 14, 15, 16, 17, 18, 19, 20, 21, 22, 23, 24 or 25 consecutive amino acids selected from wild-type sequences of Als1, Als3, HYR1, or HWP1, and an adjuvant. In certain embodiments, a composition comprising 5 to 500, 5 to 400, 5 to 300, 5 to 200 or 5 to 100 consecutive amino acids selected from wild-type sequences of Als1, Als3, HYR1, or HWP1, and an adjuvant is used as a vaccine to prevent a Candida infection in a subject. In certain embodiments a composition comprises a polypeptide comprising 5 to 500 consecutive amino acid having 80% identity or more, 85% identity or more, 90% identity or more, 95% identity or more, 96% identity or more, 98% identity or more, 99% identity or more, or 100% identity to 5 to 500 consecutive amino acids of any one of the wild-type sequences of Als1, Als3, HYR1, or HWP1, and a suitable adjuvant.

In some embodiments, a composition comprises a polypeptide comprising one or more immunogenic fragments of a polypeptide selected from the wild-type sequences of Als1, Als3, HYR1, or HWP1. Methods of identifying highly immunogenic and or highly antigenic portions of a polypeptide for use in a vaccine, and methods of making effective vaccines using portions, or all, of a polypeptide of known sequence are known in the art (e.g., as described in “Vaccinology: An Essential Guide”, by Gregg N. Milligan, and Alan D. T. Barrett, John Wiley & Sons, Dec. 4, 2014, which is incorporated herein by reference).

Any suitable adjuvant can be used for a composition or vaccine described herein. Adjuvants for use in immunogenic compositions and vaccines are known in the art and are described in, for example, Vaccine Adjuvants: Preparation Methods and Research Protocols, Derek T. O'Hagan, Springer Science & Business Media, Apr. 15, 2000; and Vaccinology: An Essential Guide, Gregg N. Milligan, Alan D. T. Barrett, John Wiley & Sons, Dec. 4, 2014, both of which are incorporated herein by reference. Non-limiting examples of adjuvants include, but are not limited to salts and amorphous materials (e.g., mineral salts), certain immunogenic serum peptides, immuno-stimulatory nucleic acids, immuno-stimulatory cytokines, plant components such as saponin-based compounds (e.g., natural and synthetic glycosidic triterpenoid compounds and pharmaceutically acceptable salts, derivatives, mimetics (e.g., isotucaresol and its derivatives) and/or biologically active fragments thereof, which possess adjuvant activity), bacterial and yeast antigens, and mammalian peptides.

Non-limiting examples of mineral salts include, but are not limited to, aluminum salts, aluminum phosphate, calcium phosphate, aluminum hydroxide (e.g., Alhydrogel), aluminum hydroxide in combination with gamma insulin (e.g., Algammulin), amorphous aluminum hydroxyphosphate (e.g., Adju-Phos), deoxycholic acid-aluminum hydroxide complex (e.g., DOC/Alum). In some embodiments, an adjuvant comprises aluminium hydroxide, aluminum phosphate and/or hydrated potassium aluminum sulfate (e.g., potassium alum).

In certain embodiments an adjuvant comprises complement factor C3d, which is a 16 amino acid peptide (See, e.g., Fearon et al., 1998, Semin. Immunol. 10: 355-61; Nagar et al., 1998, Science; 280(5367):1277-81, Ross et al. 2000, Nature Immunol., Vol. 1(2), each of which is incorporated herein by reference in its entirety). C3d is also available commercially (e.g., Sigma Chemical Company Cat. C 1547). In one embodiment, the concentration of C3d in a composition of the invention is from about 0.01 μg/mL to about 200 g/mL, preferably about 0.1 μg/mL to about 100 μg/mL, preferably about 1 μg/mL to about 50 μg/mL, more preferably about 5 μg/mL to about 20 μg/mL. It will be appreciated by one skilled in the art that the optimal C3d sequence will depend on the species to which the composition of the invention is administered.

Non-limiting examples of immuno-stimulatory nucleic acids include CpG, polyadenylic acid/poly uriddenlic acid, and Loxorbine (7-allyl-8-oxoguanosine). CpG sequences known in the art are described in U.S. Pat. No. 6,406,705, for example, which is incorporated herein by reference in its entirety. In certain embodiments, the concentration of CpG in a composition is from about 0.01 μg/mL to about 200 μg/mL, preferably about 0.1 g/mL to about 100 μg/mL, preferably about 1 μg/mL to about 50 μg/mL, more preferably about 5 μg/mL to about 20 μg/mL.

Non-limiting examples of immuno-stimulatory cytokines include interferons (e.g., interferon-gamma), interleukins (e.g., interleukin-2 (IL-2), interleukin-4 (IL-4), interleukin-6 (IL-6), interleukin-7 (IL-7), interleukin-12 (IL-12), interleukin-15 (IL-15)), colony stimulating factors, e.g., macrophage colony stimulating factors (M-CSF); G-CSF, GM-CSF), tumor necrosis factor (TNF), IL-1 and MIP-3a.

Non-limiting examples of bacterial or yeast antigens include muramyl peptides such as, but not limited to, IMMTHER™, theramide (MDP derivative), DTP-N-GDP, GMDP (GERBU adjuvant), MPC-026, MTP-PE, murametide, murapalmitine; MPL derivatives such as, but not limited to, MPL-A, MPL-SE, 3D-MLA, and SBAS-2 (i.e., mix of QS-21 and MPL-A); and mannon. Other muramyl peptides that may be used in the compositions of the invention include, but are not limited to, N-acetyl-muramyl-L-threonyl-D-isoglutamine (thr-MDP), N-acteyl-normuramyl-L-alanyl-D-isogluatme (nor-MDP), N-acetylmuramyl-L-alanyl-D-isogluatminyl-L-alanine-2-(1′-2′-dipalmitoyl-s-n-glycero-3-huydroxyphosphoryloxy)-ethylamine (MTP-PE).

Non-limiting examples of mammalian peptides that may be used in the compositions of the invention include, but are not limited to, melanonin peptide 946, neutrophil chemo-attractant peptide, and elastin repeating peptide. See, e.g., Senior et al., 1984, J Cell Bio 99 (Elastin); Needle et al., 1979, J. Biol. Chem. 254 (Neutrophil); and (Peptide 946) Cox et al. 1994, Science, 264), each of which is incorporated herein by reference in its entirety.

In some embodiments, the concentration of the adjuvant in a composition, immunogenic composition or vaccine described herein is at least 0.01% (w/v), at least 0.1% (w/v), at least 1% (w/v), at least 10% (w/v), at least 15% (w/v), at least 20% (w/v), at least 25% (w/v), or at least 30% (w/v). In some embodiments, the concentration of the adjuvant is greater than about 30% (w/v). In other embodiments, the concentration of the adjuvant compound is at least 0.1% (w/v), at least 0.5% (w/v), at least 1% (w/v), at least 5% (w/v), or at least 10% (w/v).

In some embodiments, a composition (e.g., an immunogenic composition, a vaccine) comprises a suitable buffering agent and/or a suitable salts. In some embodiments, a composition comprises a polypeptide, or immunogenic fragment thereof, an adjuvant and a pharmaceutically acceptable carrier. A composition is often aseptic and/or sterile.

In some embodiments, a pharmaceutical composition comprises an antibody that binds specifically to a Candida adhesin as described herein. In some embodiments, a pharmaceutical composition comprises an antibody that binds specifically to a Candida species.

In certain embodiments, acceptable pharmaceutical compositions are nontoxic to a recipient subject at the dosages and/or concentrations employed. A pharmaceutical composition can be formulated for a suitable route of administration. In some embodiments, a pharmaceutical composition is formulated for subcutaneous (s.c.), intradermal, intramuscular, intraperitoneal and/or intravenous (i.v.) administration. In some embodiments, the pharmaceutical composition is administered by oral or sublingual administration. In some embodiments, the pharmaceutical composition is administered for inhalation in a microparticulate formulation. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants (Remington's Pharmaceutical Sciences, 18th Ed., A.R. Gennaro, ed., Mack Publishing Company (1995) which is hereby incorporated by reference).

In certain embodiments, a pharmaceutical composition comprises a suitable excipient, non-limiting example of which include anti-adherents (e.g., magnesium stearate), binders, fillers, monosaccharides, disaccharides, other carbohydrates (e.g., glucose, mannose or dextrins), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweetners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington's Pharmaceutical Sciences, 18th Ed., A.R. Gennaro, ed., Mack Publishing Company (1995).

In some embodiments, a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agents include those having a sulfhydryl group such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments, a pharmaceutical composition comprises one or more suitable preservatives. Non limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments, a pharmaceutical composition is free of preservatives.

In some embodiments, a pharmaceutical composition is substantially free of blood components. For example, in certain embodiments, a pharmaceutical composition that comprises an antibody binding agent is substantially free of non-antibody proteins blood components (e.g., serum proteins, cells, lipids and the like). In certain embodiments where a pharmaceutical composition comprises a polyclonal antibody binding agent isolated or purified from an animal (e.g., a rabbit, sheep, goat, rodent, and the like), the composition is substantially free of non-antibody blood components derived from said animal, non-limiting examples of which include serum albumin, clotting factors, platelets, white blood cells, red blood cells, serum lipids, and the like. In some embodiments, a pharmaceutical composition is sterile. In some embodiments, a pharmaceutical composition is substantially free of endotoxin where the endotoxin component of the composition is less than 10, less than 1.0, less than 0.5, less than 0.1, less than 0.05 or less than 0.01 EU/ml. In some embodiments, a pharmaceutical composition is lyophilized to a dry powder form, which is suitable for reconstitution with a suitable pharmaceutical solvent (e.g., water, saline, an isotonic buffer solution (e.g., PBS), and the like), which reconstituted form is suitable for parental administration (e.g., intravenous administration) to a mammal.

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments, a pharmaceutical composition suitable for parental administration may contain, in addition to an antibody binding agent and/or one or more anti-fungal medications, anti-bacterial agents, and/or one or more excipients.

In some embodiments, a pharmaceutical compositions described herein may be configured for topical, rectal, or vaginal administration and may include one or more of a binding and/or lubricating agent, polymeric glycols, gelatins, cocoa-butter or other suitable waxes or fats. In some embodiments, a pharmaceutical composition described herein is incorporated into a topical formulation containing a topical carrier that is generally suited to topical drug administration and comprising any suitable material known in the art. A topical carrier may be selected so as to provide the composition in the desired form, e.g., as a solution or suspension, an ointment, a lotion, a cream, a salve, an emulsion or microemulsion, a gel, an oil, a powder, or the like. It may be comprised of naturally occurring or synthetic materials, or both. A carrier for the active ingredient may also be in a spray form. It is preferable that the selected carrier not adversely affect the active agent or other components of the topical formulation. Non-limiting examples of suitable topical carriers for use herein can be soluble, semi-solid or solid and include water, alcohols and other nontoxic organic solvents, glycerin, mineral oil, silicone, petroleum jelly, lanolin, fatty acids, vegetable oils, parabens, waxes, and the like. Semisolid carriers preferably have a dynamic viscosity greater than that of water. Other suitable vehicles include ointment bases, conventional creams such as HEB cream; gels; as well as petroleum jelly and the like. If desired, and depending on the carrier, the compositions may be sterilized or mixed with auxiliary agents, e.g., preservatives, stabilizers, wetting agents, buffers, or salts for influencing osmotic pressure and the like. Formulations may be colorless, odorless ointments, lotions, creams, microemulsions and gels.

In some embodiments, pharmaceutical compositions are formulated as creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation can be a nonionic, anionic, cationic or amphoteric surfactant.

Pharmaceutical compositions can be formulated as microemulsions, which generally are thermodynamically stable, isotropic clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives and fatty alcohols. In some embodiments, emulsifier/co-emulsifier combinations are selected from the group consisting of: glyceryl monostearate and polyoxyethylene stearate; polyethylene glycol and ethylene glycol palmitostearate; and caprylic and capric triglycerides and oleoyl macrogolglycerides. In certain embodiments a water phase includes not only water, but also, typically, buffers, glucose, propylene glycol, polyethylene glycols, for example lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG, etc.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising an antibody binding agent, with or without at least one additional therapeutic agents, can be prepared for storage by mixing the selected composition having the desired degree of purity with optional formulation agents (Remington 's Pharmaceutical Sciences, supra) in the form of a lyophilized cake or an aqueous solution. Further, in certain embodiments, a composition comprising an antibody binding agent, with or without at least one additional therapeutic agents, can be formulated as a lyophilized form (e.g., a lyophilized powder or crystalline form, a freeze-dried form) using appropriate excipients such as sucrose. In certain embodiments, a composition comprising a polypeptide, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising a polypeptide, with or without at least one additional therapeutic agents, can be formulated as a lyophilized form (e.g., a lyophilized powder or crystalline form, a freeze-dried form) using appropriate excipients such as sucrose.

In some embodiments, a carrier facilitates the incorporation of a compound into cells or tissues. For example, dimethyl sulfoxide (DMSO) is a commonly utilized carrier as it facilitates the uptake of many organic compounds into the cells or tissues of an organism. In some embodiments, a pharmaceutical carrier for a composition described herein can be selected from castor oil, ethylene glycol, monobutyl ether, diethylene glycol monoethyl ether, corn oil, dimethyl sulfoxide, ethylene glycol, isopropanol, soybean oil, glycerin, zinc oxide, titanium dioxide, glycerin, butylene glycol, cetyl alcohol, and sodium hyaluronate.

The compounds and compositions used herein can include any suitable buffers, such as for example, sodium citrate buffer and/or sequestering agents, such as an EDTA sequestering agent. Ingredients, such as meglumine, may be added to adjust the pH of a composition or antibody binding agent described herein. Antibody binding agents and compositions described herein may comprise sodium and/or iodine, such as organically bound iodine. Compositions and compounds used herein may be provided in a container in which the air is replaced by another substance, such as nitrogen. The compounds and compositions used herein can include any suitable buffers, such as for example, sodium citrate buffer and/or sequestering agents, such as an EDTA sequestering agent. Ingredients, such as meglumine, may be added to adjust the pH of a composition or a polypeptide, or immunogenic fragment thereof, described herein. Polypeptides, or immunogenic fragment thereof, and compositions described herein may comprise sodium and/or iodine, such as organically bound iodine. Compositions and compounds used herein may be provided in a container in which the air is replaced by another substance, such as nitrogen.

In certain embodiments, the optimal pharmaceutical composition will be determined by one skilled in the art depending upon, for example, the intended route of administration, delivery format and desired dosage (see e.g., Remington's Pharmaceutical Sciences, supra). In certain embodiments, such compositions may influence the physical state, stability, rate of in vivo release and rate of in vivo clearance of the antibodies of the invention. In certain embodiments, such compositions may influence the physical state, stability, rate of in vivo release and rate of in vivo clearance of the polypeptides, or portions thereof, of the invention.

### Candida Species

In some embodiments, the Candida adhesin polypeptide is derived from a Candida strain. In some embodiments, the Candida strain is Candida albicans, Candida krusei, Candida tropicalis, Candida glabrata, Candida parapsilosis, or Candida auris. In some embodiments, the Candida strain is Candida albicans. In some embodiments, the Candida strain is Candida krusei. In some embodiments, the Candida strain is Candida tropicalis. In some embodiments, the Candida strain is Candida glabrata. In some embodiments, the Candida strain is Candida parapsilosis. In some embodiments, the Candida strain is Candida auris.

The terms “Candida” as used herein refers to a genus of yeasts and is the most common cause of fungal infections worldwide Many species are harmless commensals or endosymbionts of hosts including humans; however, when mucosal barriers are disrupted or the immune system is compromised, they can invade and cause disease, known as an opportunistic infection. Candida is located on most mucosal surfaces and mainly the gastrointestinal tract, along with the skin. Candida albicans is the most commonly isolated species and can cause infections (candidiasis or thrush) in humans and other animals.

The term “candidiasis” as used herein refers to a fungal infection due to any type of Candida. Signs and symptoms of candidiasis vary depending on the area affected. Most candidal infections result in minimal complications such as redness, itching, and discomfort, though complications may be severe or even fatal if left untreated in certain populations. In healthy (immunocompetent) persons, candidiasis is usually a localized infection of the skin, fingernails or toenails (onychomycosis), or mucosal membranes, including the oral cavity and pharynx (thrush), esophagus, and the genitalia (vagina, penis, etc.); less commonly in healthy individuals, the gastrointestinal tract, urinary tract, and respiratory tract are sites of Candida infection. Common symptoms of gastrointestinal candidiasis in healthy individuals are anal itching, belching, bloating, indigestion, nausea, diarrhea, gas, intestinal cramps, vomiting, and gastric ulcers. Perianal candidiasis can cause anal itching; the lesion can be red, papular, or ulcerative in appearance, and it is not considered to be a sexually transmissible disease. Abnormal proliferation of the Candida in the gut may lead to dysbiosis. This alteration may be the source of symptoms generally described as the irritable bowel syndrome, and other gastrointestinal diseases.

### Candida Adhesin Polypeptides

In some embodiments, the Candida adhesin polypeptide is Als1, or an immunogenic fragment thereof, Als3, or an immunogenic fragment thereof, HYR1, or an immunogenic fragment thereof, or HWP1, or an immunogenic fragment thereof. In some embodiments, the Candida adhesin polypeptide is Als1, or an immunogenic fragment thereof. In some embodiments, the Candida adhesin polypeptide is Als3, or an immunogenic fragment thereof. In some embodiments, the Candida adhesin polypeptide is HYR1, or an immunogenic fragment thereof. In some embodiments, the Candida adhesin polypeptide is HWP1, or an immunogenic fragment thereof. In some embodiments, the Candida adhesin polypeptide is Als1. In some embodiments, the Candida adhesin polypeptide is Als3. In some embodiments, the Candida adhesin polypeptide is HYR1. In some embodiments, the Candida adhesin polypeptide is HWP1.

The term “Als1” as provided herein refers to the Agglutinin-like sequence protein 1, a major cell surface adhesion protein which mediates both yeast-to-host tissue adherence and yeast aggregation. Als1 acts as a downstream effector of the EFG1 regulatory pathway. It is required for rapamycin-induced aggregation of C. albicans. Als1 binds glycans and mediates adherence to endothelial and epithelial cells, thereby playing an important role in the pathogenesis of C. albicans infections. Als1 expression was associated with increased capacity of C. albicans to exacerbate disease a mouse model of DSS colitis. ALS1 gene expression was also suppressed by intestinal adaptive immune responses, and ALS1 expression was reduced in the context of NDV-3A vaccination or natural C. albicans-induced immunity in mouse models. Als1 was also targeted by intestinal IgA responses in both mouse models and in human fecal samples. Exemplary amino acid sequences for Als1 include GENBANK® Accession Nos. XP_718077.1, KAG8202526.1, and AOW30297.1, which are all incorporated herein by reference. The term “Als1” as used herein includes any of the recombinant or naturally-occurring forms of Agglutinin-like protein 1, or variants or homologs thereof that maintain Als1 activity (e.g., within at least 50%, 80%, 90%, 95%, 96%, 97%, 98%, 99% or 100% activity compared to Als1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Als1 protein.

The term “Als3” as provided herein refers to the Agglutinin-like sequence protein 3, a cell surface adhesionprotein (or invasin protein) which mediates Candida hyphal adherence and invasion to host tissues. Als3 plays an important role in the biofilm formation and pathogenesis of C. albicans infections. Als3 is necessary for C. albicans to bind to N-cadherin on endothelial cells and E-cadherin on oral epithelial cells and subsequent endocytosis by these cells. During disseminated infection, Als3 mediates initial trafficking to the brain and renal cortex and contributes to fungal persistence in the kidneys. Vaccination against the Als3 adhesin (NDV-3A vaccine) protected mice from the exacerbatory effect of C. albicans in a mouse DSS model of colitis. NDV-3A vaccinated mice also displayed decreased C. albicans adherence of colon tissue during colonization of mice. ALS3 gene expression was also suppressed by intestinal adaptive immune responses in mouse models. Als3 is also targeted by intestinal IgA responses in both mouse models and in human fecal samples. Exemplary amino acid sequences for Als3 include GENBANK® Accession Nos. AOW31402.1, XP_710435.2, and AA072959.1, which are all incorporated herein by reference. The term “Als3” as used herein includes any of the recombinant or naturally-occurring forms of Agglutinin-like sequence protein 3, or variants or homologs thereof that maintain Als3 activity (e.g., within at least 50%, 80%, 90%, 95%, 96%, 97%, 98%, 99% or 100% activity compared to Als3). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Als3 protein.

The terms “invasin” and “invasin protein” as used herein refers to a protein belonging to a class of proteins associated with the penetration of pathogens into host cells. Invasins play a role in promoting entry during the initial stage of infection.

The term “HYR1” as provided herein refers to the Hyphally regulated cell wall protein 1, a GPI-anchored hyphal cell wall protein expressed on hyphae and required for virulence. HYR1 is involved in innate immune cell evasion through confering resistance to neutrophil killing. HYR1 binds kininogen, the proteinaceous kinin precursor, and contributes to trigger the kinin-forming cascade on the cell surface. Production of kinins is often involved in the human host defense against microbial infections. Exemplary amino acid sequences for HYR1 include GENBANK® Accession Nos. KAF6069517.1, KAF6069516.1, and Q5AL03.2, which are all incorporated herein by reference. The term “HYR1” as used herein includes any of the recombinant or naturally-occurring forms of Hyphally regulated cell wall protein 1, or variants or homologs thereof that maintain HYR1 activity (e.g., within at least 50%, 80%, 90%, 95%, 96%, 97%, 98%, 99% or 100% activity compared to HYR1). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring HYR1 protein.

The term “HWP1” as provided herein refers to the Hyphal wall protein 1, a Major hyphal cell wall protein which plays a role of adhesin and is required for mating, normal hyphal development, cell-to-cell adhesive functions necessary for biofilm integrity, attachment to host, and virulence. HWP1 promotes interactions with host and bacterial molecules, thus leading to effective colonization within polymicrobial communities. HWP1 plays a crucial role in gastrointestinal colonization, in mucosal symptomatic and asymptomatic infections, in vaginitis, as well as in lethal oroesophageal candidiasis, caused by the combined action of fungal virulence factors and host inflammatory responses when protective immunity is absent. Exemplary amino acid sequences for HWP1 include GENBANK® Accession Nos. KAG8203082.1, P46593.5, AOW29115.1, and XP_709961.2, which are all incorporated herein by reference. The term “HWP1” as used herein includes any of the recombinant or naturally-occurring forms of Hyphal wall protein 1, or variants or homologs thereof that maintain HWP1 activity (e.g., within at least 50%, 80%, 90%, 95%, 96%, 97%, 98%, 99% or 100% activity compared to HWP1). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring HWP1 protein.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Use of the terms “isolated” and/or “purified” in the present specification and claims as a modifier of DNA, RNA, polypeptides or proteins means that the DNA, RNA, polypeptides or proteins so designated have been produced in such form by the hand of man, and thus are separated from their native in vivo cellular environment.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984))

The term “Als” as provided herein refers to Agglutinin-like sequences (Als) proteins. The Agglutinin-like sequence gene family encodes cell-surface glycoproteins that are involved in adhesion of fungal cells to host and abiotic surfaces. In some embodiments, the Als protein is Als1 or Als3.

### Intestinal Diseases, fungal Infections, and Treatment or Prevention of the Same

In some embodiments, the subject is suffering from, suspected of suffering from, or at risk of developing an intestinal disease. In some embodiments, the intestinal disease is inflammatory bowel disease (IBD). In some embodiments, the intestinal disease is Crohn's disease or colitis.

The term “inflammatory bowel disease” or “IBD” refers to a group of inflammatory conditions of the colon and small intestine, Crohn's disease and ulcerative colitis being the principal types. Crohn's disease affects the small intestine and large intestine, as well as the mouth, esophagus, stomach and the anus, whereas ulcerative colitis primarily affects the colon and the rectum.

The term “Crohn's disease” as used herein refers to a type of inflammatory bowel disease (IBD) that may affect any segment of the gastrointestinal tract. Symptoms often include abdominal pain, diarrhea (which may be bloody if inflammation is severe), fever, abdominal distension, and weight loss. Complications outside of the gastrointestinal tract may include anemia, skin rashes, arthritis, inflammation of the eye, and fatigue. The skin rashes may be due to infections as well as pyoderma gangrenosum or erythema nodosum. Bowel obstruction may occur as a complication of chronic inflammation, and those with the disease are at greater risk of colon cancer and small bowel cancer.

The term “ulcerative colitis” or “colitis” as used herein refers to a long-term condition that results in inflammation and ulcers of the colon and rectum. The primary symptoms of active disease are abdominal pain and diarrhea mixed with blood. Weight loss, fever, and anemia may also occur. Often, symptoms come on slowly and can range from mild to severe. Symptoms typically occur intermittently with periods of no symptoms between flares. Complications may include abnormal dilation of the colon (megacolon), inflammation of the eye, joints, or liver, and colon cancer.

The term “treating” or “treatment,” as it is used herein is intended to mean an amelioration of a clinical symptom indicative of a fungal condition. Amelioration of a clinical symptom includes, for example, a decrease or reduction in at least one symptom of a fungal condition in a treated individual compared to pretreatment levels or compared to an individual with a fungal condition, and/or an intestinal disease such inflammatory bowel syndrome, Crohn's disease, or ulcerative colitis. The term “treating” also is intended to include the reduction in severity of a pathological condition, a chronic complication or an opportunistic fungal infection which is associated with a fungal condition and/or an intestinal disease such inflammatory bowel syndrome, Crohn's disease, or ulcerative colitis. Such pathological conditions, chronic complications or opportunistic infections are exemplified below. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “preventing” or “prevention,” as it is used herein is intended to mean a forestalling of a clinical symptom indicative of a fungal condition. Such forestalling includes, for example, the maintenance of normal physiological indicators in an individual at risk of infection by a fungus or fungi prior to the development of overt symptoms of the condition or prior to diagnosis of the condition. Therefore, the term “preventing” includes the prophylactic treatment of individuals to guard them from the occurrence of a fungal condition. Preventing a fungal condition in an individual also is intended to include inhibiting or arresting the development of the fungal condition. Inhibiting or arresting the development of the condition includes, for example, inhibiting or arresting the occurrence of abnormal physiological indicators or clinical symptoms such as those described above and/or well known in the art. Therefore, effective prevention of a fungal condition would include maintenance of normal body temperature, weight, psychological state as well as lack of lesions or other pathological manifestations in an individual predisposed to a fungal condition. Individuals predisposed to a fungal condition include an individual who is immunocompromised, for example, but not limited to, an individual with AIDS, azotemia, diabetes mellitus, diabetic ketoacidosis, neutropenia, bronchiectasis, emphysema, TB, lymphoma, leukemia, or burns, or an individual undergoing chemotherapy, bone marrow-, stem cell- and/or solid organ transplantation or an individual with a history of susceptibility to a fungal condition. Inhibiting or arresting the development of the condition also includes, for example, inhibiting or arresting the progression of one or more pathological conditions, chronic complications or susceptibility to an opportunistic infection associated with a fungal condition.

The term “fungal condition” as used herein refers to fungal diseases, infection, or colonization including superficial mycoses (i.e., fungal diseases of skin, hair, nail and mucous membranes; for example, ringworm or yeast infection), subcutaneous mycoses (i.e., fungal diseases of subcutaneous tissues, fascia and bone; for example, mycetoma, chromomycosis, or sporotichosis), and systemic mycoses (i.e., deep-seated fungal infections generally resulting from the inhalation of air-borne spores produced by causal moulds; for example, zygomycosis, aspergillosis, cryptococcosis, candidiasis, histoplasmosis, coccidiomycosis, paracoccidiomycosis, fusariosis (hyalohyphomycoses), blastomycosis, penicilliosis or sporotrichosis. A fungal condition can also occur in the intestine of a subject.

### Vaccine and Adjuvants

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In some examples, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus). An exemplary infectious disease amenable to treatment with the vaccines of the invention is candidiasis. The vaccine-mediated protection can be humoral and/or cell mediated immunity induced in host when a subject is challenged with, for example, Candida adhesins or an immunogenic portions or fragments thereof.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

The term “antigen” as used herein refers to is a molecule or molecular structure that can bind to antigen receptors, including antibodies and T-cell receptors. Diverse antigen receptors are made by cells of the immune system so that each cell has a specificity for a single antigen. Upon exposure to an antigen, only the lymphocytes that recognize that antigen are activated and expanded, a process known as clonal selection. In most cases, an antibody can only react to and bind one specific antigen; in some instances, however, antibodies may cross-react and bind more than one antigen. In some embodiments, an antigen is polypeptide includes a protein selected from the group consisting of Als1, Als3, HYR1, and HWP1, or an immunogenic fragment of any of Als1, Als3, HYR1 and HWP1. In some embodiments, an antigen is polypeptide includes Als1 or an immunogenic fragment thereof. In some embodiments, an antigen is polypeptide includes Als3 or an immunogenic fragment thereof. In some embodiments, an antigen is polypeptide includes HYR1 or an immunogenic fragment thereof. In some embodiments, an antigen is polypeptide includes HWP1 or an immunogenic fragment thereof. In some embodiments, a vaccine comprises one antigen. In some embodiments, a vaccine comprises more than one antigen. In some embodiments, a vaccine comprises at least two antigens. In some embodiments, a vaccine comprises a Candida adhesin polypeptide, or immunogenic fragment thereof. In some embodiments, a vaccine comprises at least one Candida adhesin polypeptide, or immunogenic fragment thereof. In some embodiments, a vaccine comprises more than one Candida adhesin polypeptides, or immunogenic fragments thereof. In some embodiments, a vaccine comprises at least two Candida adhesin polypeptides, or immunogenic fragments thereof. In some embodiments, the vaccine comprises an Als3 polypeptide, or immunogenic fragment thereof, and an HYR1 polypeptide or immunogenic fragment thereof. In some embodiments, the vaccine comprise an Als3 polypeptide and an HYR1 polypeptide.

In certain embodiments a composition comprising a Candida adhesin polypeptide, or immunogenic fragment thereof, induces an immune response directed to the Candida adhesin polypeptide, or immunogenic fragment thereof, when the Candida adhesin polypeptide, or immunogenic fragment thereof, or a composition comprising the same, is administered to a subject. In some embodiments, an immune response is the production of one or more antibodies in a subject that bind specifically to a Candida adhesin polypeptide, or immunogenic fragment thereof. In some embodiments, an immune response is the production of one or more pro-inflammatory cytokines in a subject that are produced in response to the presence of a Candida adhesin polypeptide, or immunogenic fragment thereof. In some embodiments, an immune response is the production of one or more immunoglobulins in a subject that are produced in response to the presence of a Candida adhesin polypeptide, or immunogenic fragment thereof. In some embodiments, the immunoglobulin is an immunoglobulin A (IgA). In some embodiments, an immune response is the production of one or more antigen reactive T-cells in a subject that are produced in response to the presence of a Candida adhesin polypeptide, or immunogenic fragment thereof. The presence of an immune response to a Candida adhesin polypeptide, or immunogenic fragment thereof, can be measured by any suitable method known in the art. In certain embodiments a composition comprising a Candida adhesin polypeptide, portion or fragment thereof, is useful as a vaccine.

Candida adhesin polypeptides, and peptide fragments or variants thereof can include immunogenic epitopes, which can be identified using methods known in the art and described in, for example, Geysen et al. Proc. Natl. Acad. Sci. USA 81: 3998 (1984)). Briefly, hundreds of overlapping short peptides, e.g., hexapeptides, can be synthesized covering the entire amino acid sequence of the target polypeptide (e.g., Candida adhesin polypeptides Als1, Als3, HYR1 or HWP1). The peptides while still attached to the solid support used for their synthesis are then tested for antigenicity by an ELISA method using a variety of antisera. Antiserum against Candida adhesin polypeptides can be obtained by known techniques, Kohler and Milstein, Nature 256: 495-499 (1975), and can be humanized to reduce antigenicity, see, for example, U.S. Pat. No. 5,693,762, or produced in transgenic mice leaving an unrearranged human immunoglobulin gene, see, for example, U.S. Pat. No. 5,877,397. Once an epitope bearing hexapeptide reactive with antibody raised against the intact protein is identified, the peptide can be further tested for specificity by amino acid substitution at every position and/or extension at both C and/or N terminal ends. Such epitope bearing polypeptides typically contain at least six to fourteen amino acid residues, and can be produced, for example, by polypeptide synthesis using methods well known in the art or by fragmenting a Candida adhesin polypeptide. With respect to the molecule used as immunogens pursuant to the present invention, those skilled in the art will recognize that the Candida adhesin polypeptide can be truncated or fragmented without losing the essential qualities as an immunogenic vaccine. For example, Candida adhesin polypeptides can be truncated to yield an N-terminal fragment by truncation from the C-terminal end with preservation of the functional properties of the molecule as an immunogen. Similarly, C-terminal fragments can be generated by truncation from the N-terminal end with preservation of the functional properties of the molecule as an immunogen. Other modifications in accord with the teachings and guidance provided herein can be made pursuant to this invention to create other Candida adhesin polypeptide functional fragments, immunogenic fragments, variants, analogs or derivatives thereof, to achieve the therapeutically useful properties described herein with the native protein.

In one embodiment, the invention provides a vaccine composition having an immunogenic amount of a Candida ahdesin polypeptide, an immunogenic fragment thereof or a variant of the polypeptide. The vaccine composition also can include an adjuvant. The formulation of the vaccine composition of the invention is effective in inducing protective immunity in a subject by stimulating both specific humoral (neutralizing antibodies) and effector cell mediated immune responses against Candida ahdesin polypeptide. The vaccine composition of the invention is also used in the treatment or prophylaxis of fungal infections. The vaccine composition of the invention is also used in the treatment or prophylaxis of an intestinal disease. In some embodiments, the vaccine composition of the invention is used in the treatment or prophylaxis of inflammatory bowel syndrome. In some embodiments, the vaccine composition of the invention is used in the treatment or prophylaxis of Crohn's disease. In some embodiments, the vaccine composition of the invention is used in the treatment or prophylaxis of colitis. In some embodiments, the vaccine composition of the invention induces an immune response against Candida hyphae. In some embodiments, the vaccine composition of the invention inhibits the propagation of Candida in the intestines of the subject to which the vaccine composition is administered. In some embodiments, the vaccine composition of the invention induces IgA that reduce Candida adhesin expression and Candida tissue-association.

The vaccine of the present invention will contain an immunoprotective quantity of Candida ahdesin polypeptide antigens and is prepared by methods well known in the art. The preparation of vaccines is generally described in, for example, M. F. Powell and M. J. Newman, eds., “Vaccine Design (the subunit and adjuvant approach),” Plenum Press (1995); A. Robinson, M. Cranage, and M. Hudson, eds., “Vaccine Protocols (Methods in Molecular Medicine),” Humana Press (2003); and D. Ohagan, ed., “Vaccine Ajuvants: Preparation Methods and Research Protocols (Methods in Molecular Medicine),” Humana Press (2000).

The vaccine compositions of the invention further contain conventional pharmaceutical carriers. Suitable carriers are well known to those of skill in the art. These vaccine compositions can be prepared in liquid unit dose forms. Other optional components, e.g., pharmaceutical grade stabilizers, buffers, preservatives, excipients and the like can be readily selected by one of skill in the art. However, the compositions can be lyophilized and reconstituted prior to use. Alternatively, the vaccine compositions can be prepared in any manner appropriate for the chosen mode of administration, e.g., intranasal administration, oral administration, etc. The preparation of a pharmaceutically acceptable vaccine, having due regard to pH, isotonicity, stability and the like, is within the skill of the art.

The immunogenicity of the vaccine compositions of the invention can further be enhanced if the vaccine further comprises an adjuvant substance. Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generationn Immunological Adjuvants”, 1995, Gregoriadis G et al. (eds.), Plenum Press, New York, ISBN 0-306-45283-9, both of which are hereby incorporated by reference herein.

Preferred adjuvants facilitate uptake of the vaccine molecules by antigen presenting cells (APCs), such as dendritic cells, and activate these cells. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM® matrix); a particle; DDA (dimethyldioctadecylammonium bromide); aluminium adjuvants; DNA adjuvants; and an encapsulating adjuvant. Liposome formulations are also known to confer adjuvant effects, and therefore liposome adjuvants are included according to the invention.

The terms “adjuvant” or “immunostimulating adjuvant” are intended to mean a composition with the ability to enhance an immune response to an antigen generally by being delivered with the antigen at or near the site of the antigen. Ability to increase an immune response is manifested by an increase in immune mediated protection. Enhancement of humoral immunity can be determined by, for example, an increase in the titer of antibody raised to the antigen. Enhancement of cellular immunity can be measured by, for example, a positive skin test, cytotoxic T-cell assay, ELISPOT assay for IFN-gamma or IL-2. Adjuvants are well known in the art. Exemplary adjuvants include, for example, Freud's complete adjuvant, Freud's incomplete adjuvant, aluminum adjuvants, MF59 and QS21. In some embodiments, the adjuvant is the liposome adjuvant “CAF01” from the Statens Serum Intistut (SSI) in Denmark (Jaap T. van Dissel et al., “A novel liposomal adjuvant system, CAF01, promotes long-lived Mycobacterium tuberculosis-specific T-cell responses in human”, Vaccine, Volume 32, Issue 52, 2014, Pages 7098-7107, ISSN 0264-410X).

In addition to vaccination of subjects susceptible to fungal infections such as candidiasis, the vaccine compositions of the present invention can be used to treat, immunotherapeutically, subjects suffering from a variety of fungal infections. Accordingly, vaccines that contain one or more of Candida adhesin polynucleotides, polypeptides and/or antibody compositions described herein in combination with adjuvants, and that act for the purposes of prophylactic or therapeutic use, are also within the scope of the invention. In an embodiment, vaccines of the present invention will induce the body's own immune system to seek out and inhibit Candida adhesin molecules.

In some embodiments, the vaccine compositions of the present invention are administered by intramuscular, subcutaneous, intradermal, oral, or sublingual administration, or are administered for inhalation in a microparticulate formulation.

The term “microparticulate formulation” as used herein refers to a dosage form containing micronized drug particles that are small enough to be deposited in the lungs, such as by inhalation of a dry powder containing such micronized drug particles.

In some embodiments, the vaccine compositions of the present invention are administered only once to a subject. In some embodiments, the vaccine compositions of the present invention are administered at least once to a subject. In some embodiments, the vaccine compositions of the present inventions are administered at least twice to a subject. In some embodiments, the administering of the vaccine compositions of the present invention comprises administering a booster dose.

The term “booster dose” as used herein refers to an extra administration of a vaccine after an earlier (primer) dose. After initial immunization, a booster provides a re-exposure to the immunizing antigen. It is intended to increase immunity against that antigen back to protective levels after memory against that antigen has declined through time. For example, tetanus shot boosters are often recommended every 10 years, by which point memory cells specific against tetanus lose their function or undergo apoptosis.

In certain embodiments, a pharmaceutical composition, vaccine and/or Candida adhesin polypeptide, and peptide fragments or variants thereof, comprises aluminum as an adjuvant, e.g., in the form of aluminum hydroxide, aluminum phosphate, aluminum potassium phosphate, or combinations thereof, in concentrations of 0.05-5 mg, or from 0.075-1.0 mg, of aluminum content per dose, for example.

A person of the ordinary skill in the art has a sufficient expertise to determine the dosage of a vaccines of the instant invention. A vaccine can be administered by any suitable route, non-limiting examples of which include subcutaneously, intramuscular, intravenous, intradermal, intra-nasal, orally, sublingually, or via microparticulate formulation.

The vaccine compositions are administrated in a manner compatible with the dosage formulation and in such amount as will be prophylactically effective with or without an adjuvant. The quantity to be administered, which is generally in the range of 1 to 10 mg, preferably 1 to 1000 μg of antigen per dose, depends on the subject to be treated, capacity of the subject's immune system to synthesize antibodies, and the degree of protection desired. Precise amounts of active ingredient required to be administered can depend on the judgment of the practitioner and can be peculiar to each subject. Moreover, the amount of polypeptide in each vaccine dose is selected as an immunogenic amount which induces an immunoprotective response. In some embodiments, the dose of vaccine composition administered is in the range of 1 to 100 μg per antigen. In some embodiments, the dose of vaccine composition administered is in the range of 1 to 200 μg per antigen. In some embodiments, the dose of vaccine composition administered is in the range of 1 to 300 μg per antigen. In some embodiments, the dose of vaccine composition administered is in the range of 1 to 400 μg per antigen. In some embodiments, the dose of vaccine composition administered is in the range of 1 to 500 μg per antigen. In some embodiments, the dose of vaccine composition administered is in the range of 1 to 600 μg per antigen. In some embodiments, the dose of vaccine composition administered is in the range of 1 to 700 μg per antigen. In some embodiments, the dose of vaccine composition administered is in the range of 1 to 800 μg per antigen. In some embodiments, the dose of vaccine composition administered is in the range of 1 to 900 μg per antigen. In some embodiments, the dose of vaccine composition administered is in the range of 1 to 1000 μg per antigen. In some embodiments, the dose of vaccine composition administered is in the range of 10 to 100 μg per antigen. In some embodiments, the dose of vaccine composition administered is in the range of 1 to 200 μg per antigen. In some embodiments, the dose of vaccine composition administered is in the range of 10 to 300 μg per antigen. In some embodiments, the dose of vaccine composition administered is in the range of 10 to 400 μg per antigen. In some embodiments, the dose of vaccine composition administered is in the range of 10 to 500 μg per antigen. In some embodiments, the dose of vaccine composition administered is in the range of 10 to 600 μg per antigen. In some embodiments, the dose of vaccine composition administered is in the range of 10 to 700 μg per antigen. In some embodiments, the dose of vaccine composition administered is in the range of 1 to 800 μg per antigen. In some embodiments, the dose of vaccine composition administered is in the range of 10 to 900 μg per antigen. In some embodiments, the dose of vaccine composition administered is in the range of 10 to 1000 μg per antigen. In some embodiments, the dose of vaccine composition administered is in the range of 10 to 100 μg per antigen.

### Dosages and Products

Certain embodiments provide pharmaceutical compositions suitable for use in the technology, which include compositions where the active ingredients are contained in an amount effective to achieve its intended purpose. A “therapeutically effective amount” means an amount sufficient to prevent, treat, reduce the severity of, delay the onset of or inhibit a symptom of a Candida infection. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. The symptom can be a symptom already occurring or expected to occur. Determination of a therapeutically effective amount is well within the capability of those skilled in the art, especially in light of the detailed disclosure provided herein.

The term “an amount sufficient” as used herein refers to the amount or quantity of an active agent (e.g., an antibody binding agent, anti-fungal medication, and/or a combination of these active agents) present in a pharmaceutical composition that is determined high enough to prevent, treat, reduce the severity of, delay the onset of, or inhibit a symptom of a Candida infection and low enough to minimize unwanted adverse reactions. The exact amount of active agents or combination of active agents required will vary from subject to subject, depending on age, general condition of the subject, the severity of the condition being treated, and the particular combination of drugs administered. Thus, it is not always possible to specify an exact universal amount sufficient to prevent or treat a Candida infection for a diverse group of subjects. As is well known, the specific dosage for a given patient under specific conditions and for a specific disease will routinely vary, but determination of the optimum amount in each case can readily be accomplished by simple routine procedures. Thus, an appropriate “an amount sufficient” to prevent or treat a Candida infection in any individual case may be determined by one of ordinary skill in the art using routine experimentation.

In other embodiments, a therapeutically effective amount can describe the amount necessary for a significant quantity of the composition to contact the desired region or tissue where prevention or treatment of a Candida infection is desired.

The antibody binding agents and compositions comprising antibody binding agents as described herein can be administered at a suitable dose, e.g., at a suitable volume and concentration depending on the route of administration. Within certain embodiments of the invention, dosages of administered antibody binding agents can be from 0.01 mg/kg (e.g., per kg body weight of a subject) to 500 mg/kg, 0.1 mg/kg to 500 mg/kg, 0.1 mg/kg to 400 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In some aspects the amount of an antibody binding agent can be about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments, a therapeutically effective amount of an antibody binding agent is between about 0.1 mg/kg to 500 mg/kg, or between about 1 mg/kg and about 300 mg/kg. Volumes suitable for intravenous administration are well known.

In some embodiments, an antibody binding agent or a pharmaceutical composition comprising an antibody binding agent that is formulated for topical or external delivery can include higher amounts of an antibody binding agent. For example, pharmaceutical composition comprising an antibody binding agent that is formulated for topical administration may comprise at least 0.1 mg/ml, at least 1 mg/ml, at least 10 mg/ml, at least 100 mg/ml or at least 500 mg/ml of an antibody binding agent.

The compositions can, if desired, be presented in a pack or dispenser device, which can contain one or more unit dosage forms containing the active ingredient. The pack can for example comprise metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions comprising a compound of the invention formulated in a compatible pharmaceutical carrier can also be prepared, placed in an appropriate container, and labeled for treatment of an indicated condition.

### Kits

In some embodiments, the antibody binding agents, nucleic acids, oligonucleotide primers and/or primer pairs, compositions, polymerases, adjuvants, polypeptides, formulations, combination products and materials described herein can be included as part of kits, which kits can include one or more of pharmaceutical compositions, antibody binding agents, nucleic acids, polypeptides and formulations of the same, combination drugs and products and other materials described herein. In some embodiments, a kit comprises one or more compositions of the invention packaged into a suitable packaging material. A kit optionally includes a printed label or packaging insert that includes a description of the components and/or instructions for use in vitro, in vivo, or ex vivo, of the components therein. Exemplary instructions include instructions for a diagnostic method, treatment protocol, vaccination, or therapeutic regimen.

A kit can contain a collection of such components, e.g., two or more conjugates alone, or in combination with another therapeutically useful composition (e.g., an anti-proliferative or immune-enhancing drug). The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits can include printed labels or inserts. Printed labels or inserts include “printed matter,” e.g., paper or cardboard, or separate or affixed to a component, a kit or packing material (e.g., a box), or attached to an ampule, tube or vial containing a kit component. Inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards.

Printed labels and/or inserts can include identifying information of one or more components therein, dose amounts, clinical pharmacology of the active ingredient(s) including mechanism of action, pharmacokinetics (PK) and pharmacodynamics (PD). Printed labels and/or inserts can include information identifying manufacturer information, lot numbers, manufacturer location and date.

Printed labels and/or inserts can include information on a condition, disorder, disease or symptom for which a kit component may be used. Printed labels and/or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include printed labels or instructions for practicing any of the methods and uses of the invention described herein.

Printed labels and/or inserts can include information on any benefit that a component may provide, such as a prophylactic or therapeutic benefit. Printed labels and/or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Kits can additionally include other components. Each component of the kit can be enclosed within an individual container and all of the various containers can be within a single package. Invention kits can be designed for cold storage. Invention kits can further be designed to contain host cells expressing antibody binding agents, or that contain nucleic acids encoding antibody binding agents. The cells in the kit can be maintained under appropriate storage conditions until the cells are ready to be used.

It is understood that modifications which do not substantially affect the activity of the various embodiments of this invention are also provided within the definition of the invention provided herein. Accordingly, the following examples are intended to illustrate but not limit the present invention.

### EXAMPLES

### Example 1

### Methods and Materials

Mice

All mice used for experiments were between 6-12 weeks of age at the start of experiments. Germ free mouse experiments were either performed in sterile isolator bubbles, or in sterile techniplast cages. When using techniplast cages, mice received antibiotics water (0.5 mg/ml ampicillin, chloramphenicol, gentamycin, erythromycin), replaced every 2 weeks, to prevent bacterial contamination. Germ-free male C57Bl/6, or male Swiss Webster mice were used to quantify IgA targeting of GFP-C. albicans (YJB11522), GFP-S. cerevisiae (KOf024), or GFP-S. cerevisiae (KOf024) and iRFP-C. albicans (KOf097) over time. Germ-free male C57Bl/6 mice were used to assess IgA immune response to C. albicans (YJB11522), S. cerevisiae (KOf024), C. albicans iRFP-TetO-AHR1 (KOf204) vs NEON-SC5314, C. albicans NEON-SN250 (KOf163) vs. iRFP ahr1 Δ/Δ (KOf166), and C. glabrata Cg1. Germ-free SW females were used to assess IgA responses toward C. glabrata CBS138 and the Strope et al. 124 S. cerevisiae collection. Male and female C57Bl/6 WT (Jackson Laboratories) and TCRβ^{-/-} (Jackson Laboratories), cohoused by sex, were used to assess IgA targeting of C. albicans (YJB11522) in feces. Age-matched male C57Bl/6 WT, Rag1^{-/-}, μMT^{-/-} (Jackson Laboratories) were used to analyze intestinal NEON-C. albicans (KOf207) morphology. Age-matched germ-free male WT and Rag1^{-/-} (ULAM Germ Free Mouse Faciliy University of Michigan) mice were used for C. albicans (KOf207) RNA sequencing. Age-matched male and female C57Bl/6 WT and Rag1^{-/-} were used to compare the competitive fitness of C. albicans conditioned in germ-free mice. DSS experiments were performed on male or female C57Bl/6 mice (Jackson Laboratories). Mice colonized by fungi were gavaged 1 time with 5×10⁷ or 1×10⁸ fungal cells. Conventional mice colonized with fungi received antibiotic water (0.5 mg/ml ampicillin, chloramphenicol, gentamycin, erythromycin) for 3-14 days prior to inoculation and for the duration of the experiment. Antibiotic water was changed weekly. For experiments involving Tet-repressible C. albicans strains, anhydrotetracycline (Cayman Chemical Company) was added to drinking water at 100 μg/ml and replaced every 2 weeks for monocolonization experiments and every week for conventional mice. All mouse experiments were performed in compliance with federal regulations and guidelines set forth by the University of Utah Institutional Animal Care and Use Committee.

Human Sample Collection

Human study protocols were approved by the Institutional Review Board (HIC #1607018104) of the Yale School of Medicine, New Haven, Conn. Informed consent was obtained from all participants and/or their legal guardians and all methods were performed in accordance with relevant guidelines and regulations. Subjects with inflammatory bowel disease (either Crohn's diseases or ulcerative colitis) were identified via the EPIC electronic medical record system and all subjects resided in the state of Connecticut. Demographics, medical history and other clinical variables were collected following enrollment. A heterogeneous population of healthy subjects was recruited via advertisements on the Yale medical campus and in the New Haven Public Library. Healthy subjects were without immunodeficiencies as well as any inflammatory or other bowel disorders. All fecal samples in this study were collected at home and stored on ice packs at −20° C. prior to overnight shipment or direct drop-off the day following collection in the insulated container provided to each subject. Samples were then stored at −80° C. until use.

Fungal Strains, Media, and Growth Conditions

Fungal strains used in this study are listed in Table 4. S. cerevisiae strains expressing C. albicans and C. glabrata adhesins are listed in Table 5. Primers used for fungal strain creation and qRT-PCR are listed in Table 6. Unless otherwise stated, fungi were propagated on yeast peptone dextrose media (1% yeast extract (Fisher Scientific), 2% peptone (Fisher Scientific), 2% dextrose) at 30° C. aTC (Cayman Chemical Company) was added to a concentration of 5 μg/ml for C. albicans TetOFF strains. S. cerevisiae strains expressing C. albicans or C. glabrata adhesins were cultured in synthetic URA dropout media at 30° C. To induce C. albicans hyphal formation, strains were cultured at 37° C. in RPMI complete (RPMI 1640 with L-Glutamine [Corning] supplemented with 10% fetal bovine serum [FBS], 1×MEM Non-essential amino acids [Corning], 1 mM Sodium Pyruvate [Corning], 1×2-mercaptoethanol [Gibco]). For selection Nat resistant C. albicans transformants, nourseothricin (Jena Bioscience) was added to YPD media at 200 μg/ml. For Hyg resistant transformants, hygromycin B was added to YPD media at 600 μg/ml.

TABLE 4
Fungal strains used in this study

| Strain ID | Strain name | Source | Genotype |
|---|---|---|---|
| C. albicans | C. albicans: SN250 | 21 | wild-type |
| | | | (leu2Δ::C.d.HIS1/leu2Δ::C.m.LEU2, |
| | | | ura3Δ/URA3, his1Δ/his1Δ, |
| | | | arg4Δ/arg4Δ, iro1Δ/IRO1, MTLa/ |
| | | | MTLα) |
| GFP-S. cerevisiae | S. cerevisiae: KOf024 | this study | (RM11 genotype) ENO1-GFP-2W1S- |
| | | | OVA323-339-OVA257-264-IEα 50- |
| | | | 66-URA3/ENO1 |
| SC5314 | C. albicans: SC5314 | | |
| C. tropicalis | C. tropicalis: MYA3404 | ATCC | |
| GFP-C. albicans | C. albicans YJB11522 | 45 | pENO1-ENO1-GFP-2W1S-OVA323- |
| | | | 339-OVA257-264-IEα 50-66- |
| | | | NAT/ENO1 |
| iRFP-C. albicans | C. albicans KOf163 | this study | ENO1/ENO1::pENO1-iRFP-NAT, |
| | | | ahr1Δ::C.d.HIS1- |
| | | | ST1/ahr1Δ::C.m.LEU2-ST49, |
| | | | leu2Δ/leu2Δ, ura3Δ/URA3, |
| | | | his1Δ/his1Δ, iro1Δ/IRO1, MTLa/ |
| | | | MTLα |
| iRFP-TetO-AHR1 | C. albicans KOf204 | this study | ENO1/ENO1::pENO1-NEON- |
| | | | HYGr, TAR-FRT::tetOAHR1/TAR- |
| | | | FRT::tetO-AHR1 |
| ahr1Δ/Δ | C. albicans KOf222 | this study | ahr1Δ::aTAR-FLP-NAT/ahr1Δ::aTAR- |
| | | | FLP-NAT |
| iRFP-C. albicans | C. albicans KOf206 | this study | ENO1/ENO1::pENO1-iRFP-NAT |
| NEON-C. albicans | C. albicans KOf207 | this study | ENO1/ENO1::pENO1-NEON-NAT |
| TetO-ALS1 ahr1Δ/Δ | C. albicans KOf241 | this study | TAR-FRT::tetOALS1/TAR- |
| | | | FRT::tetOALS1 ahr1Δ::aTAR-FLP- |
| | | | NAT/ahr1Δ::aTAR-FLP-NAT |
| Cg1 | C. glabrata Cg1 | ARUP | clinical isolate |
| Cg2 | C. glabrata Cg2 | ARUP | clinical isolate |
| Cg3 | C. glabrata Cg3 | ARUP | clinical isolate |
| Cg4 | C. glabrata Cg4 | ARUP | clinical isolate |
| Cg5 | C. glabrata Cg5 | ARUP | clinical isolate |
| Cg6 | C. glabrata Cg6 | ARUP | clinical isolate |
| Cg7 | C. glabrata Cg7 | ARUP | clinical isolate |
| Cg8 | C. glabrata Cg8 | ARUP | clinical isolate |
| Cg9 | C. glabrata Cg9 | ARUP | clinical isolate |
| Cg10 | C. glabrata Cg10 | ARUP | clinical isolate |
| Cg11 | C. glabrata Cg11 | ARUP | clinical isolate |
| Cg12 | C. glabrata Cg12 | ARUP | clinical isolate |
| Cg13 | C. glabrata Cg13 | ARUP | clinical isolate |
| Cg14 | C. glabrata Cg14 | ARUP | clinical isolate |
| Cg15 | C. glabrata Cg15 | ARUP | clinical isolate |
| Cg16 | C. glabrata Cg16 | ARUP | clinical isolate |
| Cg17 | C. glabrata Cg17 | ARUP | clinical isolate |
| Cg18 | C. glabrata Cg18 | ARUP | clinical isolate |
| Cg19 | C. glabrata Cg19 | ARUP | clinical isolate |
| Cg20 | C. glabrata Cg20 | ARUP | clinical isolate |
| Cg21 | C. glabrata Cg21 | ARUP | clinical isolate |
| Cg22 | C. glabrata Cg22 | ARUP | clinical isolate |
| Cg23 | C. glabrata Cg23 | ARUP | clinical isolate |
| Cg24 | C. glabrata Cg24 | ARUP | clinical isolate |
| Cg25 | C. glabrata Cg25 | ARUP | clinical isolate |
| Cg26 | C. glabrata Cg26 | ARUP | clinical isolate |
| Cg27 | C. glabrata Cg27 | ARUP | clinical isolate |
| Cg28 | C. glabrata Cg28 | ARUP | clinical isolate |
| Cg29 | C. glabrata Cg29 | ARUP | clinical isolate |
| Cg30 | C. glabrata Cg30 | ARUP | clinical isolate |
| Cg31 | C. glabrata Cg31 | ARUP | clinical isolate |
| Cg32 | C. glabrata Cg32 | ARUP | clinical isolate |
| Cg33 | C. glabrata Cg33 | ARUP | clinical isolate |
| Cg34 | C. glabrata Cg34 | ARUP | clinical isolate |
| Cg35 | C. glabrata Cg35 | ARUP | clinical isolate |

TABLE 5
S. cerevisiae strains expressing C. albicans
or C. glabrata adhesins or adhesin-like domains

| Strain ID | C. glabrata ORF insert | fosmid | Source |
|---|---|---|---|
| SC29 | CAGL0E06688g (EPA3) | BG2ER/B2184 | |
| SC31 | CAGL0I11033g (EPA5) | B2145 | |
| SC33 | CAGL0C00110g (EPA6) | B1908 | |
| SC35 | CAGL0C05643g (EPA7) | B2154 | |
| SC37 | CAGL0H10648g (EPA17) | B2080 | |
| SC39 | CAGL0A01366g (EPA9) | centromeric | |
| SC41 | CAGL0L13299g (EPA11) | B2271 | |
| SC43 | CAGL0L13332g (EPA13) | B2271 | |
| SC45 | CAGL0E06666g (EPA2) | BG2ER/B2184 | |
| SC47 | CAGL0I11055g (EPA4) | B2145 | |
| SC49 | CAGL0E06644g (EPA1) | BG2ER/B2184 | |
| SC51 | CAGL0M00132g (EPA12) | B2159 | |
| SC58 | CAGL0A01284g (EPA10) | centromeric | |
| SC118 | CAGL0C00847g (EPA8) | centromeric | |
| SC119 | CAGL0A00099g (EPA19) | B1907 | |
| SC120 | CAGL0D06743g (EPA21) | B1933 | |
| SC121 | CAGL0I00220g (EPA23) | B2083 | |
| SC128 | CAGL0L13552g (EPA14) | B2515 | |
| SC130 | CAGL0E00275g (EPA20) | B2154 | |
| SC131 | CAGL0K00170g (EPA22) | B2148 | |
| SC229 | CAGL0J11968g (EPA15) | B2183 | |
| SC580 | CAGL0C05702g (EPA26) | B1909 | |
| SC581 | CAGL0F09295g | B1934 | |
| SC582 | CAGL0G10219g (AWP12) | B1935 | |
| SC583 | CAGL0I00209g | B2083 | |
| SC584 | CAGL0K00110g (AWP2) | B2148 | |
| SC585 | CAGL0K13024g (AWP5) | B2152 | |
| SC586 | CAGL0F09251g | B2159 | |
| SC587 | CAGL0D00143g | B1932 | |
| SC588 | CAGL0F00077g (EPA16) | B2140 | |
| SC589 | CAGL0H00209g | B2109 | |
| SC590 | CAGL0J12067g | B2183 | |
| SC591 | CAGL0B00154g | B2142 | |
| SC592 | CAGL0B05061g | B2110 | |
| SC600 | CAGL0F00099g | B2140 | |
| SC602 | CAGL0G10175g (AWP6) | B1935 | |
| SC604 | CAGL0A04851g | B2401 | |
| SC608 | CAGL0A00143g (EPA24 | B1907 | |
| SC610 | CAGL0C05687g (EPA25) | B1909 | |
| SC612 | CAGL0C00253g | B1908 | |
| SC614 | CAGL0J11935g (AWP3b) | B2183 | |
| SC616 | CAGL0I11000g | B2141 | |
| SC618 | CAGL0E00165g | B2405 | |
| SC620 | CAGL0L00157g | B2188 | |
| SC622 | CAGL0M14069g (PWP6) | B2270 | |
| SC626 | CAGL0E00231g | B2154 | |
| SC628 | CAGL0J11902g (AWP3a) | B2183 | |
| SC630 | CAGL0E00187g | B2154 | |
| SC632 | CAGL0E06600g | BG2ER/B2184 | |
| SC634 | CAGL0K13002g (AED2) | B2152 | |
| SC636 | CAGL0C00209g (AWP7) | B1908 | |
| SC687 | CAGL0I10147g (PWP1) | centromeric | |
| SC689 | CAGL0I10246g (PWP2) | centromeric | |
| SC691 | CAGL0I10200g (PWP3) | centromeric | |
| SC693 | CAGL0I10362g (PWP4) | centromeric | |
| SC695 | CAGL0I10340g (PWP5) | centromeric | |
| SC697 | CAGL0I10098g (PWP7) | centromeric | |
| Sc25 | CAGL0E06644g full-length | | 55 |
| Sc104 | CAGL0I11055g full-length | | 55 |
| Sc106 | CAGL0I11033g full-length | | 55 |
| Sc97 | CAGL0C00110g full-length | | 55 |
| Sc27 | CAGL0C05643g - full length | | 55 |
| UB2157 | CR_07070C_A (ALS3) | pBC542 + | 28 |
| (SC + ALS3) | | ALS3 | |
| UB2159 | C4_03570W_A (HWP1) | pBC542 + | 28 |
| (Sc + HWP1 | | HWP1 | |
| UB2160 | C2_09530W_A (EAP1) | pBC542 + | 28 |
| (Sc + EAP1 | | EAP1 | |
| UB2161 | C4_03520C_A (RBT1) | pBC542 + | 28 |
| (Sc + RBT1) | | RBT1 | |

TABLE 6
primers used for this study

| | | | SEQ |
|---|---|---|---|
| | | | ID |
| Primer | Sequence | Description | NO |
| KO158 | atctcattagatttggaa | Ca Cas9 FWD | 1 |
| | cttgtgggtt | | |
| KO159 | ttcgagcgtcccaaaaccttct | Ca Cas9 REV | 2 |
| KO160 | gactgtcaaggagggtattc | Ca sgRNA fwd | 3 |
| KO161 | gaataccacttgtttaccgg | Ca sgRNA rev | 4 |
| KO162 | ccgcaagtgattagacttag | Ca sgRNA nested | 5 |
| | | fwd | |
| KO163 | gaagttcctattctctagaaa | Ca sgRNA nested | 6 |
| | gt | Rev | |
| KO164 | GCTGCGCTTTATTGTTGAATaa | ahr1 KO and TetO- | 7 |
| | attaaaaatagtttacgcaagt | AHR1 sgRNA Re | |
| | c | | |
| KO165 | ATTCAACAATAAAGCGCAGCg | ahr1 KO and | 8 |
| | ttttagagctagaaatagcaa | TetO-AHR1 | |
| | g | sgRNA fwd | |
| KO166 | CCTACCTACTACTTCCTGTC | ahr1 upstream | 9 |
| | | KO check fwd | |
| KO167 | GGGTGTGGATTGAGGCATTG | ahr1 KO check | 10 |
| | | rev | |
| KO168 | ACATATAATTCTTTCATATTTTC | ahr1 Δ/Δ Nat | 11 |
| | ATTTTATTTCATACGTTAAGAT | repair fwd | |
| | CCATATCCAATAGTCGGGCCCC | | |
| | CCCTCGAGGAAGT | | |
| KO169 | CTATATCTCAAAGCGTGGAAAT | ahr1 Δ/Δ Nat | 12 |
| | ATATTCCCACTCGTCCAAAGTA | repair rev | |
| | TATAGATGTGAATTTAcgacaa | | |
| | ggtgctgaaccaaa | | |
| KO221 | GAGGCTCTTTCCTCCTCTCAA | ALS1 prom check | 13 |
| | | fwd | |
| KO222 | ACCCAAAACAGCATTCCAAG | ALS1 prom check | 14 |
| | | rev | |
| KO217 | CTCAATTGAAATGTGAAAGTaa | TetO-ALS1 sgRNA | 15 |
| | attaaaaatagtttacgcaag | rev | |
| | tc | | |
| KO218 | ACTTTCACATTTCAATTGAGgt | TetO-ALS1 sgRNA | 16 |
| | tttagagctagaaatagcaag | fwd | |
| KO219 | ATTCTATGTGGTAAAAGCATGG | TetO-ALS1 repair | 17 |
| | ACTAAATTTTCAAGTTGAGAAT | fwd | |
| | AAATCATGCATAAAGGAAGCA | | |
| | TCTCTGCACAGGAAACAGCTAT | | |
| | GAC | | |
| KO220 | ACAATGTAAATTGTTGAAGCAT | TetO-ALS1 repair | 18 |
| | CTGATATTAACAATTGGTAGTT | rev | |
| | GTTTGAACAATTCTGATGcga | | |
| | ctatttatatttgtatgtgt | | |
| | gtagg | | |
| KO193a | CGTTCCAATGAATCCAAACC | ALS4 qRT PCR fwd | 19 |
| KO194a | ACTGTCGCAGTTGCAGAAGA | ALS4 qRT PCR rev | 20 |
| KO195a | TGGAAGCTTCATCGCCTATC | ALS3 qRT PCR fwd | 21 |
| KO196a | GCGATTGAGATTGGTTGGTT | ALS3 qRT PCR rev | 22 |
| KO197a | AACAACGGTTCTGGAAGTGG | HYRI qRT PCR fwd | 23 |
| KO198 | CAGTGTGAGCACCGGTATTG | HYRI qRT PCR rev | 24 |
| KO199 | AGCTCCATCACCTGCTGTTT | ALS1 qRT PCR fwd | 25 |
| KO200 | CTGAGGTGCCTGTTGTCAAG | ALS1 qRT PCR rev | 26 |
| KO201 | TGGTCCAGGTGCTTCTTCTT | HWP1 qRT PCR fwd | 27 |
| KO202 | GGTTGCATGAGTGGAACTGA | HWP1 qRT PCR rev | 28 |
| KO134 | GCTGCCAACAATTTGGTTCT | AHR1 qRT PCR FWD | 29 |
| KO135 | TGATGGCATTGCTACCCATA | AHR1 qRT PCR Rev | 30 |
| KO092 | GTGGTACTACCATGTTCCCAGG | qRTPCR CaACT1 F | 31 |
| | | Pande et al. | |
| | | 2013 | |
| KO093 | GATAGAACCACCAATCCAGAC | qRTPCR CaACT1 R | 32 |
| | AGAG | Pande et al. | |
| | | 2013 | |
| KO029 | CGGTACCCGGGGATCTAGAAA | pKO5 in fusion | 33 |
| | GCATACTATACTATTCGACAC | | |
| | TTCCTTTCAAT | | |
| KO030 | gaaaagctGTTTAGACATTG | pKO5 in fusion | 34 |
| | GCTCTTCATTGAGCT | | |
| KO031 | TCTAAACagcttttcaatt | pKO5 in fusion | 35 |
| | caattcatcattttttt | | |
| | tttattcttttttttgattt | | |
| KO032 | GGAAAGAGttttctttcc | pKO5 in fusion | 36 |
| | attttttttttttcgtca | | |
| | attataaaaatcattacgacc | | |
| KO033 | aagaaaaCTCTTTCCATT | pKO5 in fusion | 37 |
| | GCCTTTTCTAAAGCG | | |
| KO034 | CGACTCTAGAGGATCGCAGAG | pKO5 in fusion | 38 |
| | GTTCTTACCCACTGGT | | |
| KO108 | TCCTTGGCTGGCACTGAACTCG | Forward primer | 39 |
| | | (P_{ENO1} Fw) | |
| KO109 | ATCACATGAAGTCAAATCAACT | Reverse primer | 40 |
| | TTTCTAGC | (iRFP Rv1) | |
| KO110 | CATGAGTAGCTGGCAATGAAG | Reverse primer | 41 |
| | C | (NEON Rv) | |
| TO1 | CTTTCTTTATACATATAATT | TetOAHR1 | 42 |
| | CTTTCATATTTTCATTTTAT | repair fwd | |
| | TTCATACGTTAAGATCCATA | | |
| | TCCAATAGTCGGAAACAGCT | | |
| | ATGACCATG | | |
| TO2 | GTAACGCAACCAGAACGTGTC | TetOAHR1 | 43 |
| | CGGCTGCGCTTTATTGTTGAA | repair rev | |
| | TTTAGTTTCTTCTTTGC | | |
| | CATcgactatttata | | |
| | tttgtatgtgtgtagg | | |
| KO191 | GGTGCCGTGCAAGTTTCTAT | Nat KO check rev | 44 |
| KO188 | GCGGCCGCgtttggttca | TetO integration | 45 |
| | gcaccttgtcg | check fwd | |
| TO3 | ACAGGTTGTTGTTCATCGCA | TetOAHR1 | 46 |
| | | integration | |
| | | check rev | |
| TO4 | CCTACCTACTACTTCCTGTC | AHR1 prom | 47 |
| | | check fwd | |
| KO221 | GAGGCTCTTTCCTCCTCTCAA | ALS1 prom | 48 |
| | | check fwd | |
| KO222 | ACCCAAAACAGCATTCCAAG | ALS1 prom | 49 |
| | | check rev | |
| KO030 | gaaaagctGTTTAGACATT | pKO5 integration | 50 |
| | GGCTCTTCATTGAGCT | check | |
| KO024 | GGTGCTCCAGCTAGATC | pKO5 integration | 51 |
| | | check | |
| KO031 | TCTAAACagcttttcaat | pKO5 in fusion | 52 |
| | tcaattcatcattttttt | URA3 fwd | |
| | tttattcttttttttgat | | |
| | tt | | |
| KO032 | GGAAAGAGttttctttcca | pKO5 in fusion | 53 |
| | attttttttttttcgtc | URA3 rev | |
| | attataaaaatcattacg | | |
| | acc | | |
| KO033 | aagaaaaCTCTTTCCATTGC | pKO5 in fusion | 54 |
| | CTTTTCTAAAGCG | Sc ENO1 | |
| | | term piece 2 fwd | |
| KO034 | CGACTCTAGAGGATCGCAGAG | | |
| | GTTCTTACCCACTGGT | | |
| KO010 | CCTTTAGATAATTTGTCACC | pKO5 in fusion Sc | 56 |
| | GTGGTGGAAGTTTT | ENO1 5′ fwd | |
| KO048 | CGGTACCCGGGGATCTCCAA | pKO5 in fusion Sc | 57 |
| | GTGGTTGACTG | ENO1 5′ rev | |
| KO011 | CAAATTATCTAAAGGTGAAGA | pKO5 in fusion | 58 |
| | ATTATTCACTGGTGTTGT | GFP-4peptide fwd | |
| KO012 | CAAAAGCTTCACGCGTCTCG | pKO5 in fusion | 59 |
| | AGATATCGAT | GFP-4peptide rev | |
| KO013 | CGCGTGAAGCTTTTGATTAA | pKO5 in fusion | 60 |
| | GCCTTCTAGTCCAAAAAAC | Sc ENO1 | |
| | | term piece 1 fwd | |
| KO030 | gaaaagctGTTTAGACATTGG | pKO5 in fusion | 61 |
| | CTCTTCATTGAGCT | Sc ENO1 | |
| | | term piece 1 fwd | |
| KO119 | acagggtaatatGATGTAT | pK012/13 in | 62 |
| | AGTGCTTGCTGTTCGATAT | fusion FWD | |
| | TGCTAGAG | | |
| KO120 | GGCCCGGGATCCGATATTTT | pKO12/13 in | 63 |
| | ATGATGGAATGAATGGGATG | fusion REV | |
| | AATCATCAAAC | | |

C. albicans and S. cerevisiae Strain Creation

The tetO-AHR1/tetO-AHR1 strain was made using the transient CRISPR approach. The NAT-promoter replacement cassette was amplified from pLC605 using TO1 and TO2. The sgRNA fusion cassette was made by PCR amplifying from pV1524 with KO160 and KO164 (fragment A) and KO161 and KO165 (fragment B), and then fusion PCR was performed on the fragments using the nested primers KO162 and KO163. CAS9 DNA was PCR amplified from pV1525 with KO158 and KO159. The NAT-tetO cassette (40 al), sgRNA (10 al), and CAS9 DNA (10 μl) were transformed into C. albicans SC5314 wild type. Integration was tested using TO3 and KO188. Lack of a wild-type allele was tested using TO3 and TO4. The strain was then flipped on YNB-BSA to restore NAT sensitivity.

The ahr1Δ/Δ strain was made using the transient CRISPR approach described above. The NAT replacement cassette was amplified from pLC605 using KO168 and KO169. The sgRNA fusion cassette was made as described for tetO-AHR1/tetO-AHR1. The NAT cassette, sgRNA, and CAS9 DNA were transformed into C. albicans SC5314 wild type. Upstream integration was tested using KO167 and KO188. Lack of a wild-type allele was tested using KO166 and KO167 (upstream) and also KO134 and KO135 (downstream).

The tetO-ALS1/tetO-ALS1 strain was made using the transient CRISPR approach described above. The NAT-promoter replacement cassette was amplified from pLC605 using KO219 and KO220. The sgRNA fusion cassette was made by PCR amplifying from pV1524 with KO160 and KO217 (fragment A) and KO161 and KO218 (fragment B), and then fusion PCR was performed on the fragments using the nested primers KO162 and KO163. The NAT-tetO cassette, sgRNA, and CAS9 DNA were transformed into C. albicans SC5314 wild type. Integration was tested using KO188 and KO222

Lack of a wild-type allele was tested using KO221 and KO222. The strain was then flipped on YNB-BSA to restore NAT sensitivity.

The tetO-ALS1/tetO-ALS1 ahr1Δ/Δ strain was made by repeating the ahr1Δ/Δ Nat disruption as described above in the tetO-ALS1/tetO-ALS1 strain background.

To created NEON or iRFP-expressing C. albicans strains, pENO1-iRFP or NEON vectors were digested with NotI and transformed into C. albicans strains. Integration of NEON was confirmed by PCR using KO108 and KO110, and iRFP confirmed by PCR using KO108 and KO109.

GFP-S. cerevisiae KOf024 strain was created by digesting pKO5 with sphI and sacI and transforming into RM11 (MATα lys2 Δ 0 ura3 Δ 0) and selected on synthetic URA dropout media. Integration of ENO1-GFP-4peptide-ENO1term-URA3 was confirmed by PCR using KO030 and KO024. Positive transformant were then crossed with RM11 (MAT a leu2 Δ 0) to create a prototrophic KOf024 GFP+RM11 strain.

Vector Creation

pKO5 was created using In-Fusion HD Cloning Plus kit (Takara Bio). 5 inserts were amplified using the primers listed in Table 6 and cloned into pUC19. RM11 gDNA used as templates for following PCR reactions: KO033 and KO034; KO010 and KO048; KO013 and KO030. URA3 cassette was amplified using KO031 and KO032 from pML43⁴⁴. GFP-4peptide was amplified from M4366.

pKO12 and pKO13 (Table 7) were created using In-Fusion HD Cloning Plus kit (Takara Bio). HYG was amplified from pYM70⁴⁶ using KO119 and KO120 and inserted into EcoRV digested NAT pENO1-NEON and pENO1-iRFP plasmids.

TABLE 7
Plasmids used in this study

| Vector name | Description | Use | Source |
|---|---|---|---|
| pKO5 | Sc 3′ENO1-GFP-4peptide- | GFP expression in | This study |
| | ENO1term-URA3 | S. cerevisiae | |
| pKO12 | Candida pENO1-iRFP670- | Hyg iRFP expression | This study |
| | HygR | in C. albicans | |
| pKO13 | Candida pENO1-NEON- | Hyg NEON expression in | This study |
| | HygR | in C. albicans | |
| | ENO1-NEON Candida | Nat iRFP expression in | Seman et al.* |
| | | C. albicans | |
| | ENO1-RFP Candida | Nat NEON expression in | Seman et al.* |
| | | C. albicans | |
| pYM70 | Ca-Hyg | Source of Hyg for | Basso et al. ** |
| | | C. albicans | |
| | | disruption strains | |
| pV1524 | CaCas9/gRNA Solo entry | Source of Cas9 and | Vyas et al. *** |
| | plasmid | sgRNA sequence for | |
| | | C. albicans | |
| | | TetO and | |
| | | disruption strains | |
| pLC605 | Candida vector with TetO | Source of TAR-FLP-NAT and | Veri et al. **** |
| | NATr with FLP | TAR-FRT-Nat-TetO | |

*Seman, B. G. et al. Yeast and filaments have specialized, independent activities in a zebrafish model of Candida albicans infection. Infection and Immunity (2018). doi:10.1128/IAI.00415-18.
** Basso, L. R. et al. Transformation of Candida albicans with a synthetic hygromycin B resistance gene. Yeast 27, 1039-1048 (2010).
*** Vyas, V. K. et al. New CRISPR Mutagenesis Strategies Reveal Variation in Repair Mechanisms among Fungi. mSphere 3, (2018).
**** Veri, A. O. et al. Tuning Hsf1 levels drives distinct fungal morphogenetic programs with depletion impairing Hsp90 function and overexpression expanding the target space. PLOS Genetics 14, e1007270 (2018).

Processing of Fecal or Intestinal Samples for Total or Fungal Binding Antibodies, and Assessing Fungal Burden

Feces or intestinal contents were suspended in sterile, cold PBS to a concentration of 100 mg/ml-500 mg/ml. Samples were homogenized by breaking up solid material with a pipette tip, followed by 1-2 min of vortexing. Homogenate was used for quantifying in vivo antibody binding to fungi using flow cytometry. Fungal burden was quantified by diluting 1/10- 1/10⁴ with sterile PBS and plated on YPD media. To quantify total and fungal binding IgA in vitro, samples were spun at 13,000×g for 10 min and cleared supernatant saved. Mouse IgA was quantified from 1/10- 1/10⁴ dilutions of intestinal was using the Ready-SET-Go mouse IgA ELISA kit (Thermo Scientific).

Imagining C. albicans in Feces or in the Gut

For imaging in the gut, intestinal sections were placed in tissue embedding cassette (Fisher Scientific) and samples fixed for 3-4 hr in Carnoy's Solution (Spectrum Chemical) at room temperature with shaking. Cassettes were then washed 2 times for 5 min with cold PBS, and then for 40 min in 40% EtOH. Samples were stored in 70% EtOH until sectioning by the ARUP Research histology lab. To stain intestinal sections, slides were deparaffined in Coplin Staining Jars (VWR): two 6 min washes in xylenes followed sequential 2 min washes in 100% EtOH, 100% EtOH, 95% EtOH, 70% EtOH, and 40% EtOH. Slides were incubated in humidified staining chamber at room temperature for 20 min with 150 μl blocking buffer (PBS with 4% donkey serum). Slides were stained with 150 μl 1/500 dilution of AF488 anti-Candida antibody (Meridian) in humified staining chamber overnight at 4° C. Slides were washed twice for 5 min in PBS supplemented with 0.1% tween-20 with shaking. Slides rinsed 2 times in cold PBS before mounting and imaging.

To image C. albicans (KOf207) from fecal or intestinal contents, material homogenized to 100 mg/ml in PBS and 10 μl placed in 96-well V-bottom plate. Samples were incubated in 100 μl blocking buffer at 4° C. for 20 min, and then stained at 4° C. with 100 μl 1/500 AF488 anti-Candida antibody for 20 min. Cells were washed twice with 150 μl PBS and fixed with 2% paraformaldehyde solution (Fisher Scientific). Staining of C. albicans using the anti-Candida antibody dramatically amplified the brightness of C. albicans cells over simply using the NEON fluorescent marker expressed by the C. albicans strain used for these imaging studies.

All samples were mounted using Vectashield HardSet Antifade Mounting Medium (Vector) and images were taken at the University of Utah Imaging Core using a Nikon AIR Confocal microscope. Imaging analysis done using Fiji as described in Schindelin, J. et al. Fiji: An open-source platform for biological-image analysis, Nature Methods (2012).

RNA Isolation, qRT-PCR, and RNAseq

Feces or intestinal contents (250-500 mg) were harvested from mice, immediately frozen on dry ice, and then stored at −80° C. until RNA isolation. RNA was isolated using the RNAeasy mini kit (Qiagen). For qRT-PCR experiments, cDNA was synthesized using qScript cDNA synthesis kit (Quanta Biosciences), and C. albicans transcripts were quantified using PowerUp SYBR Green Master Mix (Applied Systems). Primers used for qRT-PCR are listed in Table 6. All transcripts were compared to C. albicans ACT1.

For RNAseq of C. albicans (KOf207) colonizing germ-free WT and Rag1−/− mice, RNA was isolated from cecal contents after 4 weeks of colonization. NEBNext Ultra II Directional RNA library pep kit and the NEBNext Poly(A) mRNA Magnetic Isolation Module (NEB) were used to generate mRNA-seq libraries according to the manufacturer's directions. Each sample's library (n=9) was barcoded with NEB provided oligos and libraries were multiplexed before sequencing. Multiplexed libraries were sequenced on a single lane of a HiSeq 2500 with paired-end 125 Cycle sequencing by The University of Utah Genomics Core facility, a part of the Health Sciences Cores at the University of Utah.

Raw Illumina fastq sequences were first quality-trimmed and adapter-filtered using trim_galore's implementation of cutadapt (M. Martin, Cutadapt removes adapter sequences from high-throughput sequencing reads. EMBnet.journal, [S.l.], v. 17, n. 1, p. pp. 10-12, May 2011. ISSN 2226-6089, hereby incorporated by reference in its entirety). Sequences were trimmed when quality score dropped below 20 and any remaining sequences with length less than 20, or where the mate-pair did not pass quality checks was discarded. Quality filtered sequences were then aligned against the mouse transcriptome (GRCm38) with Bowtie2 to remove host reads. Sequence pairs which did not align concordantly to the mouse reference were then used as input to align against C. albicans reference. On average 66%+/−4% (mean+/−SE) of reads mapped to the host transcriptome. Reads were mapped against the current C. albicans SC5314 transcriptome reference (Assembly 22, candidagenome.org; M. S. Skrzypek et al., The Candida Genome Database (CGD): incorporation of Assembly 22, systematic identifiers and visualization of high throughput sequencing data, Nucleic Acids Research, Volume 45, Issue D1, January 2017, Pages D592-D596) using the “_default_coding” version which contains a haploid complement of all coding features without introns. Kallisto (version 0.45.0) was used to map and quantify transcript abundances and resulted in a final average of 6.7 million (+/−1.1 million) read pairs mapping per sample (as described in Bray, N. L., Pimentel, H., Melsted, P. & Pachter, L. Near-optimal probabilistic RNA-seq quantification. Nature Biotechnology 34, 525-527 (2016)). The “chromosomal_feature.tab” file provided by candidagenome.org for the assembly 22 version was used to create a map file to correlate each transcript to a gene, and also replace the systematic gene names implemented in Assembly 22 with common/standard gene names where they were available. The Sleuth R package was then used to read in the Kallisto read quantification files and aggregate mapped transcripts by gene, providing the gene name map file that was created before (as described in Pimentel, H., Bray, N. L., Puente, S., Melsted, P. & Pachter, L. Differential analysis of RNA-seq incorporating quantification uncertainty. Nature methods 14, 687-690 (2017)). Sleuth was further used to perform gene-level differential expression testing between the 2 mouse genotypes with Wald's test. All code used for processing and mapping reads is described by Kyla Ost, et al. (Ost, K. S., O'Meara, T. R., Stephens, W. Z. et al. Adaptive immunity induces mutualism between commensal eukaryotes. Nature 596, 114-118 (2021). GO-term enrichment analysis was performed on transcripts that were at least 2-fold (≥1 LOG2) differentially regulated between groups with q<0.05 using the “clusterProfiler” package in R and an organism annotation package created with the “AnnotationForge” R package from the NCBI-hosted genome (see Yu, G., Wang, L. G., Han, Y. & He, Q. Y. ClusterProfiler: An R package for comparing biological themes among gene clusters. OMICS A Journal of Integrative Biology 16, 284-287 (2012)). The R package “fgsea” was used to compare published hyphal-upregulated gene set (as described in Witchley, J. N. et al. Candida albicans Morphogenesis Programs Control the Balance between Gut Commensalism and Invasive Infection. Cell Host and Microbe 25, 432-443.e6 (2019)) with the ranked list of differentially expressed genes identified with Sleuth (Sergushichev, A. A. An algorithm for fast preranked gene set enrichment analysis using cumulative statistic calculation. bioRxiv 060012 (2016). doi:10.1101/060012). Genes at least 2-fold induced (Log₂≥1) in hyphal-inducing conditions were classified as hyphal-upregulated. RNAseq volcano plots using EnhancedVolcano (K. Blighe, S. Rana, M. Lewis, 2021-10-29; bioconductor.org/packages/release/bioc/vignettes/EnhancedVolcano/inst/doc/EnhancedVolca no.html#references).

Quantification of IgA Binding to Cultured Fungi In Vivo

500 μl-1 ml of fecal or intestinal homogenate was filtered through 40 μm or 70 μm into 50 ml conical tube. Filters were rinsed with 10 ml cold PBS, filters were discarded, and then samples were spun at 4000 rpm for 5 min. Supernatants were discarded, and pellets were resuspended in 10 ml PBS and spun at 4000 rpm for 5 min. Supernatants were discarded and pellets were vortexed in the residual PBS by vortexing. 10 μl of each sample was pipetted into 96-well V-bottom plate. A well was prepared for an unstained control and IgA isotype control. 200 μl 10% fetal bovine serum (v/v) in PBS was added to each sample, and incubated on ice for 10 min. Samples spun down at 3000 rpm for 5 min, and the samples were stained with 100 μl 1/250 anti mouse IgA PE (eBioscience clone mA-6E1) g/ml calcofluor white (CFW) (Sigma-Aldrich) in column buffer (PBS supplemented with 10 mM HEPES [Corning] 2 mM EDTA [Corning], and 0.5% [v/v] fetal bovine serum [GIBCO BRL]). The isotype control sample was similarly stained, but with 1/500 Rat IgG1 K Isotype Control PE. Samples were analyzed on the BD LSR Fortessa and data analyzed by FlowJo.

Imaging Flow Cytometry of C. albicans in Feces

Fecal samples from SW mice that had been monocolonized with GFP-C. albicans (YJB11522) for 3 weeks were used for imaging. Fecal samples were prepared and stained for CFW and IgA as described for flow cytometry analysis. Samples from 3 mice were analyzed using the Amnis ImageStream Mk II using the 488 nm, 405 nm, and 592 nm laser. Data was analyzed using IDEAs 6.3 software. C. albicans were gated on GFP+ and CFW intermediate populations, and finally gated by IgA. Brightfield images of C. albicans populations were visually inspected to exclude non-C. albicans fecal particles. Data from the 3 mice were combined into a single file to analyze C. albicans circularity by IgA binding. Circularity of the IgA+ and IgA− populations were calculated using the CFW channel using the Shape Change wizard.

Assessing In Vitro IgA Binding to Cultured Fungi

Cultured fungi were normalized to OD600=1-3 in PBS supplemented with 1% bovine serum albumin (Sigma-Aldrich) and 0.01% Sodium Azide (P/B/A). 25 μl of cultured fungi were mixed with 25 μl cleared intestinal wash in 96-well V-bottom plates and incubated on ice, or at 4° C. for 45 min.-1 hr. Samples were spun at 3000 rpm for 5 min, and washed 2 times with 150 μl P/B/A. Samples were stained in the dark at 4° C. with anti-mouse IgA (eBioscience clone mA-6E1) diluted 1/500 in column buffer (PBS supplemented with 10 mM HEPES [Corning] 2 mM EDTA [Corning], and 0.5% [v/v] fetal bovine serum [GIBCO BRL]). An isotype control, stained with 1/500 Rat IgG1 K Isotype Control PE, was included for each fungus tested. Samples were washed 2 times with 150 μl column buffer and IgA binding was quantified using either a BD LSR Fortessa or BD Celesta. IgA binding intensity was normalized to the isotype negative control for each sample.

Screening Noble and Homann Mutant Collections for IgA Binding

Intestinal wash from small intestinal contents pooled from 4 male C57Bl/6 mice monocolonized with C. albicans (YJB11522) for 25 days, or whole intestinal contents from female SW mice monocolonized with C. albicans (YJB11522) for 60 days were used. For both intestinal wash samples, contents were homogenized in PBS to 100 mg/ml, cleared by spinning at 5000 rpm for 15 min, and then filtered through 0.22 m filter.

The Homann and Noble and C. albicans homozygous deletion collections were purchased from the Fungal Genetics Stock Center (http://www.fgsc.net/). The Noble collections were described in Noble, S. M., French, S., Kohn, L. A., Chen, V. & Johnson, A. D., “Systematic screens of a Candida albicans homozygous deletion library decouple morphogenetic switching and pathogenicity,” Nat. Genet. 42, 590-598 (2010), and the Homann collections were described in Homann, O. R., Dea, J., Noble, S. M. & Johnson, A. D., “A phenotypic profile of the Candida albicans regulatory network,” PLoS Genet. 5, e1000783 (2009), both of which are incorporated by reference herein in their entirety. Mutant collections were cultured overnight in round-bottom 96-well plates in 200 μl of YPD. Each strain was normalized to an OD600=3 in sterile-filtered PBS/1% BSA/0.01% Azide (P/B/A). 25 μl of each strain was then mixed with 25 μl intestinal wash in 96-well V-bottom plates and incubated for 50 min on ice. Samples were spun at 3000 rpm for 5 min, and washed twice with 150 μl P/B/A. Samples were stained in the dark for 20 min on ice with 50 μl anti-mouse IgA PE (eBioscience clone mA-6E1), diluted 1:500 in column buffer (PBS supplemented with 10 mM HEPES [Corning] 2 mM EDTA [Corning], and 0.5% [v/v] fetal bovine serum [GIBCO BRL]), and then washed twice with 150 μl column buffer. Each collection contains an isogenic background WT strains used as a positive control. As a negative control, the WT strains were incubated with intestinal wash, but then stained with the PE isotype control antibody (Rat IgG1 K Isotype Control PE eBioscience) at a 1:500 dilution. For the Homann screen, IgA binding was quantified using the BD FACSCanto Analyzer using the 96 well high throughput sampler. The geometric mean IgA binding was quantified using FlowJo and binding intensity was divided by the geometric mean intensity of the isotype control. For the Noble collection, IgA binding was quantified using the BD Celesta using the 96 well high throughput samples. When quantifying IgA binding intensity by FlowJo, we noticed that there was significant plate-to-plate variability in average IgA binding intensity. We therefore normalized binding intensity by dividing the geometric mean IgA binding intensity for each sample by the average geometric mean IgA value for corresponding plate. For both collections, normalized IgA binding intensity were averaged between duplicate samples, and then IgA binding Z-scores were calculated by the following: Z=(normalized IgA binding−average normalized IgA binding for whole collection)/(standard deviation of normalized IgA binding for the collection). For the Homann collection, both wells of the orf19.610Δ/Δ (encoding EFG1) mutant were contaminated and was excluded from the figures and tables in this study. We isolated a pure culture of efg1Δ/Δ and quantified IgA binding using the same protocol, finding no difference in IgA targeting compared to the WT strain. For the Noble collection, we did not acquire data from three mutant strains (orf19.191, orf19.1041, and orf19.6124) because the cells were lost during the processing of the samples.

Assessment of Lamina Propria and Peyer Patch Lymphocyte Populations

Lamina propria cells were isolated as described previously (in Kubinak, J. L. et al. MyD88 signaling in T cells directs IgA-mediated control of the microbiota to promote health. Cell host & microbe 17, 153-63 (2015), which is incorporated by reference herein in its entirety) with the following alterations: The epithelial dissociation step was performed with 10 ml HBSS (without Mg²⁺ and Ca²⁺) containing 30 mM 0.5 EDTA (Corning), 10 mM HEPES (Corning), and 1.5 mM DL-Dithirothreitol (DTT) (Sigma). Cells were dissociated for 30 min at 37° C. with shaking at 37° C. for 30 min. Digestions were carried out as in 53, but instead of percoll separation, digestion solution was spun at 800×g for 10 min, and washed one time in 10 ml cold PBS. Digests were resuspended in 5 ml RPMI complete (RPMI 1640 with L-Glutamine [Corning] supplemented with 10% fetal bovine serum [FBS], 1×MEM Non-essential amino acids [Corning], 1 mM Sodium Pyruvate [Corning], 1×2-mercaptoethanol [Gibco]), and counted. Peyer patches were isolated and prepared as described previously in Kubinak et al.

5×10⁵ to 10⁶ live cells were stained for flow cytometry. All cells were first stained for with Ghost Dye Violet 510 (Tonbo) viability stain following Ghost dye protocol. Extracellular and intracellular antibody staining was performed as described previously 53 using antibodies and concentrations listed in Table 8. Data was collected using a BD LSR Fortessa and analyzed using FlowJo software. All cell populations were first gated by FSC and SSC to exclude cellular debris, then gating out doublets, and finally gating for live (Ghost negative) cell populations. Gates and analysis for all stains were set using fluorescent minus one (all antibodies except for one) controls for each antibody.

TABLE 8
Flow cytometry antibodies used in this study

| Target | | | | Staining | Intra or |
|---|---|---|---|---|---|
| (mouse) | Conjugate | Clone | Company | dilution | extracellular |
| CD138 | PE | 281-2 | BioLegend | 1:250 | Extra |
| (Syndecan-1) | | | | | |
| CD19 | PerCP/Cy5.5 | 6D5 | BioLegend | 1:250 | Extra |
| CD19 | PerCP/Cy5.5 | 1D3 | Tonbo | 1:250 | Extra |
| CD278 | FITC | 7E.17G9 | eBioscience | 1:250 | Extra |
| (ICOS) | | | | | |
| CD279 (PD-1) | PE/Cy7 | RMP1-30 | BioLegend | 1:250 | Extra |
| CD3e | Brilliant violet 711 | 125-2c11 | BIoLegend | 1:250 | Extra |
| CD4 | violetFluor 450 | GK1.5 | Tonbo | 1:250 | Extra |
| CD45 | violetFluor 450 | 30-F11 | Tonbo | 1:250 | Extra |
| CD95 (Fas) | PE/Cy7 | Jo2 | BD Pharmingen | 1:250 | Extra |
| Foxp3 | PerCP/Cy5.5 | FJK-16S | eBioscience | 1:50 | Intra |
| Foxp3 | APC | FJK-16S | eBioscience | 1:50 | Intra |
| GL7 | Alexa Fluor 488 | GL-7 | eBioscience | 1:250 | Extra |
| IFNγ | Brilliant Violet | XMG1.2 | BioLegend | 1:50 | Intra |
| | 605 | | | | |
| IgA | PE | mA-6E1 | eBioscience | 1:250 | Extra |
| IgA | FITC | mA-6E1 | Invitrogen | 1:100 | Intra |
| IgD | Alexa Fluor 647 | 11-26c.2a | BioLegend | 1:250 | Extra |
| IL-17A | PE/Cy7 | eBio17B7 | eBioscience | 1:50 | Intra |
| RORyt | PE | Q31-378 | BD Bio | 1:50 | Intra |
| Rat IgG1 K | PE | eBRG1 | eBioscience | 1:250 | Extra |
| Isotype | | | | | |
| Control | | | | | |

C. albicans Cell Wall Isolation and Western Blot

C. albicans (SC5314) was culture overnight in 5 ml YPD at 30° C. 100 μl of culture added to either 5 ml YPD and rotated overnight at 30° C. (yeast) or 5 ml RMPIc and shook 150 rpm overnight at 37° C. (hyphae). Yeast and hyphal cultures were harvested and washed once with 10 ml cold 10 mM Tris HCl pH 7.4, resuspended in 1 ml 10 mM Tris HCl, and stored in bead beating tubes at −80° C. Fungal cell wall were isolated as described in 54 Cell walls were normalized to 20 mg/ml in Tris HCl pH 7.4, and 50 μl of each sample was mixed with 10 μl 6× Laemmli sample buffer and boiled for 10 min. 30 μl (500 μg) was loaded and ran on two 4-20% 12-well Mini-PROTEAN TGX precast protein gels (BioRad). For one gel, proteins transferred to a 0.45 nm nitrocellulose membrane, stained with intestinal wash from C57Bl/6 mice monocolonized with C. albicans (cleared small intestinal wash diluted 100 μl in 5 ml TBST with 5% dried milk [v/v]), followed by staining with 1/1000 dilution of goat anti-mouse IgA (chain) HRP secondary antibody (SouthernBiotech) diluted in TBST with 5% milk. The duplicate protein gel was stained with Coomassie. 2 protein gel regions >254 KDa were cut from each lane were submitted to the Mass Spectrometry and Proteomics Core Facility at the University of Utah for protein identification using the following procedure:

Digestion of in-gel proteins: Gel bands were first destained with 50 mM ammonium bicarbonate in 50:50 water:methanol. Proteins were reduced with DTT for 45 minutes at 60° C. and then alkylated with IAA for 30 minutes at room temperature in the dark. Gel spots were washed three times in 50 mM ammonium bicarbonate in water for 45 minutes/wash cycle. Gel spots were cut into small pieces and dehydrated using 100% acetonitrile. Proteins were digested overnight at 38° C. with Trypsin/LysC mixture. One μg pf trypsin was used per sample. The digestion was quenched by acidification with 1% formic acid to a pH of 2-3. Peptides were extracted from the gel using 50% acetonitrile/1% formic acid and then concentrated en vacuo to a final volume of 5 μL.

LC/MS/MS Analysis: Reversed-phase nano-LC/MS/MS was performed on an Eksigent Ekspert nanoLC 425 system (SciEx) coupled to a Bruker MAXIS ETD II QToF mass spectrometer equipped with a nanoelectrospray source. Concentrated samples were diluted with a 1:1 ratio of sample:0.1% formic acid in water. Five μL of the samples were injected onto the liquid chromatograph. A gradient of reversed-phase buffers (Buffer A: 0.2% formic acid in water; Buffer B: 0.2% formic acid in acetonitrile) at a flow rate of 150 μL/min at 60° C. was set-up. The LC run lasted for 83 minutes with a starting concentration of 5% buffer B increasing to 55% over the initial 53 minutes and a further increase in concentration to 95% over 63 minutes. A 15 cm long/100 μm inner diameter nanocolumn was employed for chromatographic separation. The column was packed, in-house, with reverse-phase BEH C18 3.5 μm resin (Xbridge). MS/MS data was acquired using an auto-MS/MS method selecting the most abundant precursor ions for fragmentation. The mass-to-charge range was set to 350-1800.

Analysis of MS/MS Data: Mascot generic format (MGF) files were generated from the raw MS/MS data. Mascot (version 2.6) uses the MGF file for database searching and protein identification. For these samples a custom database was searched with Candida albicans taxonomy selected. The parameters used for the Mascot searches were: trypsin digest; two missed cleavages; carbamidomethylation of cysteine set as fixed modification; oxidation of methionine were set as variable modifications; and the maximum allowed mass deviation was set at 11 ppm.

Conditioning of C. albicans in WT and Rag1^{-/-} C57Bl/6 Germ-Free Mice and Competitive Colonization

Colonization of mice: To assess the competitive fitness of C. albicans conditioned in WT or Rag1^{-/-} C57Bl/6 germ-free mice, fecal samples from monocolonized mice (iRFP KOf206 in Rag1^{-/-} and NEON KOf207 in WT) were homogenized at 2 days or 4 weeks post inoculation to 100 mg/ml and equal volumes combined. Quantitative culture showed similar fungal burden in WT and Rag1^{-/-} mice at both 2 days and 4 weeks post colonization (2-day averages: Rag1^{-/-} 2.15×10⁶ cfu/g, WT 6.30×10⁶ cfu/g, and 4 week averages: Rag1^{-/-} 5.50×10⁶ cfu/g, WT 6.80×10⁶ cfu/g). Antibiotic-treated WT and Rag1^{-/-} recipient mice (see Mice section for antibiotic treatment protocol) were gavaged with 100 μl of the combined fecal homogenate. 100 μl of the combined homogenate was also plated on YPD and cultured at 30° C. 4 days post inoculation, fecal samples from each recipient mouse was homogenized in 1 ml sterile PBS and the entire sample cultured on YPD plates at 30° C. Cultured iRFP and NEON C. albicans strains were also competed in WT and Rag1^{-/-} mice to control for inherent competitive colonization differences. Cultured KOf206 and KOf207 were mixed 1:1 and 10⁸ cells were gavaged into antibiotic-treated WT and Rag1^{-/-} recipient mice. 100 μl of the inoculum, and homogenized fecal samples collected 4 days post colonization were plated on YPD and cultured overnight at 30° C.

Calculating normalized competitive index. C. albicans cultured from inoculum, or from fecal samples were scraped and homogenized in 5 ml sterile H₂O. Suspended C. albicans were diluted and relative numbers of iRFP (Rag1^{-/-} conditioned) and NEON (WT conditioned) were quantified using flow cytometry using a BD LSR Fortessa and FlowJo analysis. Competitive index for iRFP- or NEON strains were calculated using the following formula described in Noble, S. M., French, S., Kohn, L. A., Chen, V. & Johnson, A. D. Systematic screens of a Candida albicans homozygous deletion library decouple morphogenetic switching and pathogenicity. Nature genetics 42, 590-8 (2010), which is incorporated by reference herein in its entirety:

(Recovered count/total recovered count)/(inoculum count/total inoculum count).

We noted that the iRFP strain had a slight competitive colonization disadvantage compared to the NEON strain from culture at 4 days post inoculation (NEON CI=1.3 and iRFP CI=0.71 in WT mice, and NEON CI=1.15 and iRFP CI=0.85 in Rag1^{-/-} mice). To account for this, we divided the CI values calculated from the competition of intestinal conditioned strains by the CI values calculated from the competition from culture. Assessing human fecal antibody binding to cultured fungi

Human fecal samples were homogenized in sterile PBS to a concentrations of ˜100 mg/ml by disruption by pipette tip and vortexing for 2 min. Tubes were spun at 13000×g for 15 min and supernatants were stored in 100 μl aliquots at −80° C. Total IgA was quantified by ELISA (coating SAB3701393, Decection ab97215, and standards were human IgA1 and IgA2 (Athens Research & Technology). Sample were normalized to between 1 ug/ml-9 ug/ml total IgA before probing cultured fungi. IgA, IgG, and IgM binding to S. cerevisiae (RM11), C. albicans (SC5314), C. glabrata (Cg1), and C. tropicalis (MYA3404) was performed as described in Quantification of IgA binding to cultured fungi in vivo.

Samples were stained for IgM (Anti-IgM Fc5p Goat Polyclonal Antibody AF488 Jackson ImmunoResearch), IgG (Goat Anti-Human IgG Antibody AF594 Jackson ImmunoResearch), and IgA (Goat Anti-Human IgA α Antibody AF647 Jackson ImmunoResearch). Each antibody was diluted according to the manufacture recommendations and used at 1/500 dilution. Staining intensity was quantified using a BD LSR Fortessa and analyzed by FlowJo. Geometric mean staining intensity was normalized between fungi so that the unstained controls (stained without fecal IgA but with the fluorescent secondaries) had the same baseline staining intensity.

Investigating 124 S. cerevisiae Strain Collection for IgA Induction in Germ-Free Mice

The 124 Strope et. al S. cerevisiae collection (as described in Strope, P. K. et al. The 100-genomes strains, an S. cerevisiae resource that illuminates its natural phenotypic and genotypic variation and emergence as an opportunistic pathogen. Genome Research 25, 762-774 (2015)) purchased from the Fungal Genetics Stocks center (http://www.fgsc.net/). Germ free male and female Swiss Webster mice were used to investigate the IgA response to the Strope et. al 124 S. cerevisiae strain collection. Individual cultured strains were normalized by OD600 and combined into 6 pools of 20-24 strains. Mice were gavaged with 1-3×10⁸ cells every week for 3 weeks. All mice were kept in sterile techniplast cages and kept on antibiotics water (500 mg/L ampicillin, chloramphenicol, gentamycin, erythromycin) to prevent bacterial contamination. Cecal contents were used to assess IgA binding of S. cerevisiae in vivo, quantify total IgA levels, and IgA binding to the 20-24 strains present in the pool colonizing the mice (See Assessing in vitro IgA binding to cultured fungi above.)

DSS Colitis Experiments

Mice were given 2.5% dextran sodium sulfate salt (M.W 36000-50000 colitis grade MP Biomedicals) for 8 days. Mice were weighed daily and sacrificed on day 8. Colons were removed, cleared of fecal material, measured, fixed in 10% buffered formalin (Fisher Chemical) for 1-2 days at room temperature, and stored 70% EtOH. ARUP Research histology lab sectioned colons and performed H&E staining. Colon damage score was scored as described in Kubinak, J. L. et al. MyD88 signaling in T cells directs IgA-mediated control of the microbiota to promote health. Cell host & microbe 17, 153-63 (2015), which is incorporated by reference herein in its entirety.

NDV-3A Vaccination

NDV-3A or alum vaccination of GF or conventionally colonized mice (SPF mice) was performed as described previously (in Singh, S. et al., The NDV-3A vaccine protects mice from multidrug resistant Candida auris infection. PLoS Pathog. 15, e1007460 (2019), which is incorporated by reference herein in its entirety), although with just one boost. Anti-Als3 IgA and IgG was assessed in the faeces using an ELISA as also described previously in Singh, S. et al. See FIG. 13A for a diagram of the vaccination experiments.

Statistical Analysis

Figure creation and statistical analysis was performed with Prism 8 software. Specific statistical tests are indicated in figure legends

### Example 2

### Intestinal IgA Targets Candida Species

The reactivity of intestinal IgA to four commensal fungi Candida glabrata, Candida albicans, Saccharomyces cerevisiae and Candida tropicalis—was tested using human faecal samples. Most samples contained fungal-reactive antibodies and IgA dominated the response (FIG. 5A). One form of IBD, Crohn's disease, is associated with serum antibodies called ASCAs (anti-Saccharomyces cerevisiae antibodies), which target cell-wall components in Saccharomyces and Candida species, and therefore we compared ASCAs in our samples. IBD status did not affect the levels of fungal-reactive IgA or IgA ASCAs in the faeces (FIGS. 5C-5F), which is in contrast to the elevated levels of ASCAs observed in the serum and an increased reactivity to S. cerevisiae in patients with Crohn's disease (FIGS. 5C-5F). Although not altered by IBD, IgA was significantly less reactive towards S. cerevisiae and most reactive towards C. albicans (FIG. 1A, FIG. 5B). Together, these results suggest that homeostatic intestinal IgA targets specific members of the fungal community, and that serum and mucosal Ig responses are distinct.

Mono-association of germ-free (GF) mice with C. albicans induced a specific IgA response in both C57BL/6 and Swiss Webster (SW) mice, which was characterized by the binding of IgA to C. albicans in the faeces, the induction of total and C. albicans-specific IgA, increased numbers of IgA plasma cells in the colon and increased numbers of Peyer's patch lymphocytes, which include germinal centre B cells (GC B cells). By contrast, IgA and immune cell populations in mice colonized with S. cerevisiae were similar to those in GF mice (FIGS. 1B-1D, FIGS. 6A-6F). Colonization with C. albicans, but not S. cerevisiae, induced a serum IgA and/or IgG1 response (FIGS. 6G and 6H), despite similar colonization levels (FIG. 6I). A collection of 124 strains of S. cerevisiae that was screened for the induction of IgA through pooled colonization of GF mice still failed to induce IgA (FIGS. 7A-7C). C. albicans is not unique in its ability to induce intestinal IgA responses, as C. glabrata also induced total and C. glabrata-specific IgA in addition to Peyer's patch GC B cells and T follicular helper (T_{FH}) cells (FIGS. 6J-6M). C. albicans- and C. glabrata-induced IgA was species-specific and did not cross-react with other species in vitro (FIG. 1F, FIG. 6N). In addition, the binding of IgA to C. albicans was significantly reduced in T-cell-deficient mice (TCRβ^{-/-} mice) relative to wild-type mice (FIG. 6O), suggesting that the C. albicans-induced IgA is T-cell-dependent. Together, these data demonstrate that several different fungi induce intestinal IgA responses in humans and mice, but that the response is species-dependent.

### Example 3

### IgA Targets Adhesive Fungal Effectors

To study how T-cell-dependent IgA responses influence C. albicans, RNA was isolated from monocolonized wild-type or T- and B-cell-deficient Rag1^{-/-} mice for RNA sequencing (RNA-seq). Despite similar colonization between the genotypes (FIG. 8A), 25% of C. albicans genes were differentially regulated (q<0.05) between wild-type and Rag1-mice (FIG. 1G, Supplementary Table 1), and were enriched for genes that regulate pathogenesis, symbiosis and adhesion (FIG. 1G, FIGS. 8B and 8C). Virulence factors, such as the ALS adhesins, candidalysin (ECE1) and the SAP proteases, which promote tissue invasion and damage in disseminated infection models, were upregulated in Rag1^{-/-} mice14. Carbohydrate and amino acid transporters were significantly downregulated in Rag1−/− mice (FIGS. 8C and 8D), suggesting that nutrient acquisition may be altered by adaptive immune responses. Notably, many of the upregulated genes in Rag1^{-/-} mice are known to be specifically expressed during the C. albicans yeast-to-hyphal morphological transition, in which C. albicans forms elongated hyphae that are specialized for adhesion and tissue invasion (see Noble, S. M., Gianetti, B. A. & Witchley, J. N. Candida albicans cell-type switching and functional plasticity in the mammalian host. Nat. Rev. Microbiol. 15, 96-108 (2017)). Indeed, gene set enrichment analysis (GSEA) revealed a significant enrichment of hyphal-associated genes in the Rag1^{-/-} mice (FIG. 1H; normalized enrichment score (NES)=2.94 and Padj=3.6×10−4), together suggesting that adaptive immune responses suppress the expression of genes associated with C. albicans hyphae. Imaging of C. albicans morphology revealed an increase in the proportion of C. albicans hyphal cells in Rag1^{-/-} mice (FIG. 1I, FIG. 8E). Control of C. albicans morphology was dependent on B cells, as B-cell-deficient μMT^{-/-} (MT is also known as Ighm) mice (FIG. 8F) are also unable to suppress C. albicans hyphae (FIG. 1J). Imaging flow cytometry (ImageStream) revealed that elongated hyphae dominated the IgA-bound population in the faeces. Furthermore, IgA⁺ fungal cells were quantifiably less circular, or yeast-shaped, than the IgA⁻ population (FIG. 1K). Thus, IgA targets C. albicans hyphae and intestinal B cell responses temper expression of this morphotype in the gut.

It was shown here that intestinal IgA preferentially binds to specific fungi, and previous studies have revealed a similar phenomenon for gut bacteria (see Weis, A. M. & Round, J. L. Microbiota-antibody interactions that regulate gut homeostasis. Cell Host Microbe 29, 334-346 (2021)). However, for both bacteria and fungi, little is known regarding the identity and function of epitopes that are targeted by IgA. To identify C. albicans genes that are required for IgA targeting, we screened two homozygous deletion mutant collections for strains with reduced IgA binding (as described in Noble, S. M., French, S., Kohn, L. A., Chen, V. & Johnson, A. D. Systematic screens of a Candida albicans homozygous deletion library decouple morphogenetic switching and pathogenicity. Nat. Genet. 42, 590-598 (2010); and Homann, O. R., Dea, J., Noble, S. M. & Johnson, A. D. A phenotypic profile of the Candida albicans regulatory network. PLoS Genet. 5, e1000783 (2009)).

This analysis identified 13 strains with a reduction in IgA binding. The ahr1 Δ/Δstrain (adhesion and hyphae regulator 1) and other identified mutants correspond to transcription factors that promote C. albicans adhesion and biofilm formation18 (FIG. 2A, 2B, Supplementary Table 2). Notably, adhesion and biofilm formation are also central characteristics of hyphae, highlighting adhesion as a key process targeted by intestinal IgA responses.

The role of filamentation and adhesion in the induction of IgA was tested using a yeast-locked strain (TetOn-NRG1) that constitutively expresses the Nrg1 transcription factor; this strain blocks filamentation unless treated with anhydrotetractyline (aTC) to repress the expression of NRG1 (FIG. 9A) (see Braun, B. R., Kadosh, D. & Johnson, A. D. NRG1, a repressor of filamentous growth in C. albicans, is down-regulated during filament induction. EMBO J. 20, 4753-4761 (2001)). The ahr1 Δ/Δ strain, which has defects in adhesion but is still capable of hyphal formation, was also used. TetOn-NRG1 remained locked in the yeast state in monocolonized mice, and the ahr1 Δ/Δ strain produced hyphae to a similar level to wild-type C. albicans (FIG. 9B). Analysis of IgA immune responses revealed that both TetOn-NRG1 and ahr1 Δ/Δ induced significantly less intestinal IgA, with a trend towards fewer plasma cells in the colon, and fewer Peyer's patch GC B cells and T_{FH} cells, despite colonizing the intestine to a similar extent to wild-type C. albicans (FIG. 2C, FIGS. 9C-9F). The function of Ahr1 in IgA stimulation was confirmed using a TetOff-AHR1 strain (FIGS. 9G-9K). These data suggest that both Ahr1 and hyphae express molecules responsible for the induction of IgA immune responses.

Cell-surface adhesin proteins mediate C. albicans hyphae adherence to host tissue and Ahr1 directly promotes the expression of adhesin genes. Notably, a key difference between C. glabrata and S. cerevisiae, which are otherwise genetically similar, is that C. glabrata encodes a large group of adhesin genes that facilitate host tissue association. To test the hypothesis that fungal adhesins are targeted by IgA, cell-wall protein fractions from yeast and hyphal C. albicans were probed with intestinal IgA from mice that were monocolonized with C. albicans. An increase in IgA binding to a 245-kDa molecular weight region was detected in the hyphal cell-wall fraction. Liquid chromatography with tandem mass spectrometry (LC-MS/MS) analysis identified the Als3 adhesin as the most abundant protein specific to the hyphal fraction (FIG. 2E, Supplementary Table 3). In addition, quantitative PCR (qPCR) analysis revealed that the ALS1 adhesin was significantly reduced in expression in both the ahr1 Δ/Δ and the yeast-locked TetOn-NRG1 C. albicans strains (FIG. 2D, FIG. 9L), together implicating Als1 and Als3 as adhesin targets of IgA. In line with this, constitutive expression of ALS1 (TetOn-ALS1 ahr1Δ/Δ) increased IgA binding to ahr1Δ/Δ cells to a level even above that of the wild type, but repression of ALS1 (TetOff-ALS1 ahr1Δ/Δ) reduced IgA binding compared to the wild type (FIG. 9M). Leveraging a collection of S. cerevisiae strains engineered to express C. albicans adhesins on the cell surface24 (FIG. 10A), we observed that expression of C. albicans Als1, Als3 and Hwp1 adhesins was sufficient to promote IgA binding (FIG. 2F). Similarly, one predicted C. glabrata adhesin was recognized by C. glabrata-induced IgA (FIG. 10B). The Als1 and Als3 adhesins are also directly targeted by human faecal IgA (FIG. 2G, FIG. 9N), demonstrating that C. albicans adhesins are the dominant IgA epitopes. Of note, only Als1-expressing S. cerevisiae was able to induce IgA and IgG (FIG. 1I), indicating that although multiple adhesins are direct targets of the host IgA response, only specific adhesins are sufficient to induce antibody-promoting immune responses.

### Example 4

### Adaptive Immunity Improves Candida Fitness

GF Rag1^{-/-} and wild-type mice were colonized with isogenic C. albicans strains expressing different fluorescent markers (called conditioned strains) to test whether adaptive immunity influences the colonization of C. albicans. Wild-type- and Rag1^{-/-} conditioned C. albicans were then competed in a group of C. albicans-naive wild-type or Rag1^{-/-} mice (FIG. 3A). C. albicans strains conditioned for four weeks in wild-type mice had a significant competitive advantage over C. albicans conditioned in Rag1^{-/-} mice (FIG. 3B). Supporting that IgA-which takes at least seven days to induce—is involved, this competitive advantage was lost when strains are conditioned for only two days (FIG. 3B). Notably, the wild-type-conditioned C. albicans fitness advantage did not require T and B cells in the recipient mice (FIG. 3B), and this fitness advantage persisted until 14 days (FIG. 12). To test a role for hyphae during immune conditioning, the yeast-locked TetOn-NRG1 strain was used to repeat the four-week conditioning and competition experiment. In contrast to the wild-type C. albicans, immune conditioning of yeast-locked C. albicans did not increase its competitive fitness (FIG. 3C), indicating that the immune-mediated advantage depends on the ability to undergo the yeast to hyphal transition. In support of a role for adhesins in this process, constitutive expression of ALS3 (TetOn-ALS3) significantly reduced competitive fitness compared to the wild type, whereas repression of ALS3 expression (TetOff-ALS3) rescued competitive fitness (FIG. 3D). Together, these data suggest that expression of effectors enriched on hyphae are detrimental to the competitive fitness of C. albicans.

### Example 5

### Immune-Targeted Adhesins Worsen Colitis

C. albicans and other Candida species are associated with a number of inflammatory diseases, including IBD, so we used the dextran sulfate sodium (DSS)-induced colitis model to test a role for filamentation during disease (FIG. 12A) using vehicle (no C. albicans), wild-type C. albicans, yeast-locked C. albicans (TetOn-NRG1) and hyphal-locked C. albicans (TetOff-NRG1). Wild-type C. albicans and hyphal-locked C. albicans significantly exacerbated colon damage (FIG. 4A) whereas colitis was ameliorated in mice treated with the yeast-locked (TetOn-NRG1) strain (FIGS. 4A and 4B), indicating that hyphae worsen colitis. The adhesin-deficient ahr1 Δ/Δ strain and the TetOff-AHR1 strain induced significantly less colon damage than wild-type C. albicans and the TetOn-AHR1 strain, respectively (FIG. 4C, FIG. 13B). Constitutive expression of ALS1 in the ahr1 Δ/Δ background (TetOn-ALS1 ahr1 Δ/Δ) significantly increased colon damage compared to the ahr1 Δ/Δ mutant, and this increase was reversed to ahr1 Δ/Δ levels upon repression of ALS1 expression (TetOff-ALS1 ahr1 Δ/Δ) (FIG. 4C, FIG. 13C.) These data suggest that IgA-targeted hyphal cells and the Als1 adhesin exacerbate colitis.

To determine whether induction of an adhesin-specific immune response could prevent C. albicans-associated damage during colitis, the anti-Candida NDV-3A vaccine was used to immunize mice. NDV-3A induces an Als3-specific immune response that has been shown to be effective in preventing recurrent vaginal yeast infections in a human phase Ib/IIa trial (see Edwards, J. E. Jr et al. A fungal immunotherapeutic vaccine (NDV-3A) for treatment of recurrent vulvovaginal candidiasis—a phase 2 randomized, double-blind, placebo-controlled trial. Clin. Infect. Dis. 66, 1928-1936 (2018).).

Vaccination with NDV-3A induced faecal Als3-reactive IgG and IgA in GF mice (FIG. 14), which bound C. albicans hyphae (FIG. 4D, FIG. 14F). Vaccination did not affect C. albicans morphology or intestinal lumen fungal burden (FIGS. 14D, 14E, 14G and 14H). However, NDV-3A vaccination reduced issue-associated C. albicans in the colon (FIG. 4E) and reduced the expression of C. albicans ALS1 (FIG. 4F). Notably, NDV-3A vaccination prevented C. albicans-associated damage in mice with colitis (FIG. 4G, FIGS. 14I-14L). These data show that adaptive immune responses can be harnessed against a C. albicans adhesin to reduce C. albicans-associated damage during colitis.

Our studies reveal that host adaptive immune responses represent a force that promotes an expression program within commensal fungi that licenses their mutualism. For C. albicans, pathogenic hyphae and hyphae-associated virulence factors have been shown to be less fit for gut colonization. Here we find that adaptive immune responses target and select against these cell types in the gut, improving their general commensal fitness. This example highlights a potential positive feedback loop between host and fungus that maintains homeostasis. This phenomenon may not be specific to interactions with C. albicans, as C. glabrata also provokes an adhesin-specific IgA response. Our study also provides a foundation to develop clinical interventions to restore homeostasis during disease. Human IgA deficiency is associated with IBD, although not associated with altered Candida levels (see Fiedorová, K. et al. Bacterial but not fungal gut microbiota alterations are associated with common variable immunodeficiency (CVID) phenotype. Front. Immunol. 10, 1914 (2019)), may increase colitogenic effector expression.

We have identified at least one antigen, Als1, as the first to our knowledge—specific C. albicans effector that has been shown to contribute to intestinal colitis. Als1, and related adhesins (for example, Als3, which is around 84% identical to Als1 at the amino acid level (see Spellberg, B. J. et al. Efficacy of the anti-Candida rAls3p-N or rAls1p-N vaccines against disseminated and mucosal candidiasis. J. Infect. Dis. 194, 256-260 (2006)), are important virulence factors that promote mucocutaneous and disseminated infection. Using a clinically tested Als-based vaccine, we show that adhesin-specific immune responses can prevent intestinal disease and that vaccination strategies can be used to enhance commensal processes that already occur naturally. Altogether, these data reveal a mutualistic interaction between eukaryotes that comprises a bidirectional communication circuit involving fungal colonization factors and host immunity.

### Discussion

A number of studies have focused on immune responses against fungi at extra-intestinal sites. However, many of these fungi, including C. albicans and C. glabrata, are proficient colonizers of mammalian mucosal tissues that are constantly monitored by the adaptive immune system. Our studies demonstrate host adaptive immune responses represent a force that promotes an expression program within commensal fungi that licenses their mutualism. We also demonstrate that vaccination against immunodominant fungal epitopes restores mutualistic host/fungal interactions. For C. albicans, effectors that increase fitness within the gut are surprisingly divergent from fungal effectors required for pathogenesis. C. albicans hyphae are important for driving disseminated disease and represent a perpetual threat in the gut. Previous studies have shown that certain types of yeast cells have a competitive advantage in this environment, and that mechanisms may exist for monitoring and clearing excess hyphae.

The present data show that C. albicans expression of hyphae and hyphal-associated pathogenic effectors is reduced by adaptive immune responses. This requires B cells and is associated with selective targeting of hyphal cells with IgA, suggesting that IgA may be selecting against, or suppressing hyphae. Though we acknowledge that other T/B-cell dependent immune molecules may also modulate C. albicans morphology. A recent study performed experimental evolution of C. albicans by performing 10-one-week serial passages through mice and saw no difference in the evolution of C. albicans in Rag^{-/-} mice³². This led them to conclude that adaptive immune responses were not important for sculpting fungal commensalism. However, our data demonstrate that the IgA responses against fungal commensals requires long-term colonization and does not begin to develop robustly until after 7 days of colonization and peaks at 20-30 days. Consistent with these previous studies, we find that suppression of hyphae and hyphal effectors is associated with increased competitive fitness, even when tested in mice that lack T and B cells. This reveals a positive feedback loop, in which the host targets pathogenic effectors on C. albicans, thus indirectly benefiting C. albicans competitive colonization. Many of these fungal commensals will form life-long associations with their mammalian host, with disease occurring in only a minority of people. The present data demonstrate that an antigen-specific IgA response may be a critical mediator of this interaction, which functions to promote a symbiotic relationship between host and fungus.

An important aspect of this work is how the interaction between host and fungi influence mammalian health. IgA deficiency is the most common immunodeficiency in humans and is associated with altered bacterial community composition and inflammatory bowel disease. A recent study analyzed the fungal composition in individuals that were IgA deficient and found no difference in the structure of the fungal community. However, our data suggests that adaptive immune responses go beyond selecting for presence or absence of certain fungi to promote communication between fungi and host. We demonstrate that a Candida adhesin based vaccine, which has been clinically tested in humans, effectively prevents C. albicans-associated tissue damage during colitis. We have identified at least one antigen, Als1, as the first specific C. albicans effector shown to contribute to intestinal colitis. Als1, and related adhesins, are important virulence factors that promote mucocutaneous and disseminated infection⁴². Als-specific immunity can protect against disease and an anti-C. albicans vaccine in human clinical trials is based on the Als3 adhesin 43. Using this vaccine, we have demonstrated that enhancing immune responses against these adhesins prevents intestinal disease without necessarily clearing the fungi. Indeed, there is no difference in fungal load between mock and vaccinated animals. This suggests that vaccination strategies can be employed to enhance already naturally occurring commensal processes. Taken together, these data reveal a mutualistic interaction between eukaryotes that involves a bidirectional communication circuit involving fungal colonization factors and host immunity.

### REFERENCES

1. Witchley, J. N. et al. Candida albicans Morphogenesis Programs Control the Balance between Gut Commensalism and Invasive Infection. Cell Host and Microbe 25, 432-443.e6 (2019).
2. Zhai, B. et al. High-resolution mycobiota analysis reveals dynamic intestinal translocation preceding invasive candidiasis. Nature Medicine 26, 59-64 (2020).
3. Chiaro, T. R. et al. A member of the gut mycobiota modulates host purine metabolism exacerbating colitis in mice. Science Translational Medicine 9, eaaf9044 (2017).
4. Leonardi, I. et al. CX3CR1+mononuclear phagocytes control immunity to intestinal fungi. Science 359, 232-236 (2018).
5. Iliev, I. D. et al. Interactions between commensal fungi and the C-type lectin receptor Dectin-1 influence colitis. Science 336, 1314-7 (2012).
6. Bacher, P. et al. Human Anti-fungal Th17 Immunity and Pathology Rely on Cross-Reactivity against Candida albicans. Cell 176, 1340-1355.e15 (2019).
7. Noverr, M. C., Falkowski, N. R., McDonald, R. A., McKenzie, A. N. & Huffnagle, G. B. Development of Allergic Airway Disease in Mice following Antibiotic Therapy and Fungal Microbiota Increase: Role of Host Genetics, Antigen, and Interleukin-13. Infection and Immunity 73, 30-38 (2005).
8. Limon, J. J. et al. Malassezia Is Associated with Crohn's Disease and Exacerbates Colitis in Mouse Models. Cell Host & Microbe 0, (2019).
9. Huertas, B. et al. Serum Antibody Profile during Colonization of the Mouse Gut by Candida albicans: Relevance for Protection during Systemic Infection. Journal of Proteome Research 16, 335-345 (2017).
10. Doron, I. et al. Human gut mycobiota tune immunity via CARD9-dependent induction of anti-fungal IgG antibodies. Cell 184, 1017-1031.e14 (2021).
11. Standaert-Vitse, A. et al. Candida albicans Is an Immunogen for Anti-Saccharomyces cerevisiae Antibody Markers of Crohn's Disease. Gastroenterology 130, 1764-1775 (2006).
12. Walker, L. J. et al. Anti-Saccharomyces cerevisiae antibodies (ASCA) in Crohn's disease are associated with disease severity but not NOD2/CARD15 mutations. Clinical and Experimental Immunology 135, 490-496 (2004).
13. Bunker, J. J. et al. Innate and Adaptive Humoral Responses Coat Distinct Commensal Bacteria with Immunoglobulin A. Immunity 43, 541-553 (2015).
14. Roetzer, A., Gabaldón, T. & Schuller, C. From Saccharomyces cerevisiae to Candida glabrata in a few easy steps: Important adaptations for an opportunistic pathogen. FEMS Microbiology Letters 314, 1-9 (2011).
15. Strope, P. K. et al. The 100-genomes strains, an S. cerevisiae resource that illuminates its natural phenotypic and genotypic variation and emergence as an opportunistic pathogen. Genome Research 25, 762-774 (2015).
16. Jiang, T. T. et al. Commensal Fungi Recapitulate the Protective Benefits of Intestinal Bacteria. Cell Host and Microbe (2017). doi:10.1016/j.chom.2017.10.013
17. Bunker, J. J. et al. Natural polyreactive IgA antibodies coat the intestinal microbiota. Science (New York, N.Y.) 358, eaan6619 (2017).
18. Richardson, J. P., Ho, J. & Naglik, J. R. Candida-Epithelial Interactions. Journal of Fungi 4, 22 (2018).
19. Noble, S. M., Gianetti, B. A. & Witchley, J. N. Candida albicans cell-type switching and functional plasticity in the mammalian host. Nature Reviews Microbiology 15, 96-108 (2017).
20. Macpherson, A. J. S. et al. IgA production without mu or delta chain expression in developing B cells. Nature immunology 2, 625-31 (2001).
21. Noble, S. M., French, S., Kohn, L. A., Chen, V. & Johnson, A. D. Systematic screens of a Candida albicans homozygous deletion library decouple morphogenetic switching and pathogenicity. Nature genetics 42, 590-8 (2010).
22. Homann, O. R., Dea, J., Noble, S. M. & Johnson, A. D. A Phenotypic Profile of the Candida albicans Regulatory Network. PLoS Genetics 5, e1000783 (2009).
23. Lohse, M. B., Gulati, M., Johnson, A. D. & Nobile, C. J. Development and regulation of single- and multi-species Candida albicans biofilms. Nature Reviews Microbiology 16, 19-31 (2018).
24. Braun, B. R., Kadosh, D. & Johnson, A. D. NRG1, a repressor of filamentous growth in C. albicans, is down-regulated during filament induction. EMBO Journal 20, 4753-4761 (2001).
25. De Groot, P. W. J., Bader, O., De Boer, A. D., Weig, M. & Chauhan, N. Adhesins in Human Fungal Pathogens: Glue with Plenty of Stick. (2013). doi:10.1128/EC.00364-12
26. Ruben, S. et al. AHR1 and TUP1 contribute to the transcriptional control of virulence-associated genes in Candida albicans. mBio 11, (2020).
27. Askew, C. et al. The zinc cluster transcription factor Ahr1p directs Mcm1p regulation of Candida albicans adhesion. Mol Microbiol 79, 940-953 (2011).
28. Nobbs, A. H., Margaret Vickerman, M. & Jenkinson, H. F. Heterologous Expression of Candida albicans Cell Wall-Associated Adhesins in Saccharomyces cerevisiae Reveals Differential Specificities in Adherence and Biofilm Formation and in Binding Oral Streptococcus gordonii. EUKARYOTIC CELL 9, 1622-1634 (2010).
29. Xu, Z. et al. De novo genome assembly of Candida glabrata reveals cell wall protein complement and structure of dispersed tandem repeat arrays. Molecular Microbiology (2020). doi:10.1111/mmi.14488
30. Zupancic, M. L. et al. Glycan microarray analysis of Candida glabrata adhesin ligand specificity. Molecular Microbiology 68, 547-559 (2008).
31. Carreté, L. et al. Patterns of Genomic Variation in the Opportunistic Pathogen Candida glabrata Suggest the Existence of Mating and a Secondary Association with Humans. Current Biology 28, 15-27.e7 (2018).
32. Tso, G. H. W. et al. Experimental evolution of a fungal pathogen into a gut symbiont. Science 362, 589-595 (2018).
33. Sokol, H. et al. Fungal microbiota dysbiosis in IBD. Gut 66, 1039-1048 (2017).
34. Uppuluri, P. et al. Human Anti-Als3p antibodies are surrogate markers of NDV-3A vaccine efficacy against recurrent vulvovaginal candidiasis. Frontiers in Immunology 9, (2018).
35. Spellberg, B. J. et al. Efficacy of the Anti-Candida rAls3p—N or rAls1p—N Vaccines against Disseminated and Mucosal Candidiasis. The Journal of Infectious Diseases 194, 256-260 (2006).
36. Coleman, D. A., Oh, S. H., Manfra-Maretta, S. L. & Hoyer, L. L. A monoclonal antibody specific for Candida albicans Als4 demonstrates overlapping localization of Als family proteins on the fungal cell surface and highlights differences between Als localization in vitro and in vivo. FEMS Immunology and Medical Microbiology 64, 321-333 (2012).
37. Pande, K., Chen, C. & Noble, S. M. Passage through the mammalian gut triggers a phenotypic switch that promotes Candida albicans commensalism. Nature genetics 45, 1088-91 (2013).
38. Chen, C., Pande, K., French, S. D., Tuch, B. B. & Noble, S. M. An iron homeostasis regulatory circuit with reciprocal roles in Candida albicans commensalism and pathogenesis. Cell Host and Microbe 10, 118-135 (2011).
39. White, S. J. et al. Self-Regulation of Candida albicans Population Size during GI Colonization. PLoS Pathogens 3, e184-e184 (2007).
40. Pierce, J. V & Kumamoto, C. A. Variation in Candida albicans EFG1 expression enables host-dependent changes in colonizing fungal populations. mBio 3, e00117-12 (2012).
41. Fiedorovi, K. et al. Bacterial but not fungal gut microbiota alterations are associated with common variable immunodeficiency (CVID) phenotype. Frontiers in Immunology (2019). doi:10.3389/fimmu.2019.01914
42. Ibrahim, A. S. et al. Vaccination with Recombinant N-Terminal Domain of Als1p Improves Survival during Murine Disseminated Candidiasis by Enhancing Cell-Mediated, Not Humoral, Immunity. Infection and Immunity 73, 999-1005 (2005).
43. Schmidt, C. S. et al. NDV-3, a recombinant alum-adjuvanted vaccine for Candida and Staphylococcus aureus, is safe and immunogenic in healthy adults. Vaccine (2012). doi:10.1016/j.vaccine.2012.10.038
44. Voth, W. P. Yeast vectors for integration at the HO locus. Nucleic Acids Research (2001). doi:10.1093/nar/29.12.e59
45. Igyirt6, B. Z. et al. Skin-Resident Murine Dendritic Cell Subsets Promote Distinct and Opposing Antigen-Specific T Helper Cell Responses. Immunity 35, 260-272 (2011).
46. Basso, L. R. et al. Transformation of Candida albicans with a synthetic hygromycin B resistance gene. Yeast 27, 1039-1048 (2010).
47. Seman, B. G. et al. Yeast and filaments have specialized, independent activities in a zebrafish model of Candida albicans infection. Infection and Immunity (2018). doi:10.1128/IAI.00415-18
48. Schindelin, J. et al. Fiji: An open-source platform for biological-image analysis. Nature Methods (2012). doi:10.1038/nmeth.2019
49. Bray, N. L., Pimentel, H., Melsted, P. & Pachter, L. Near-optimal probabilistic RNA-seq quantification. Nature Biotechnology 34, 525-527 (2016).
50. Pimentel, H., Bray, N. L., Puente, S., Melsted, P. & Pachter, L. Differential analysis of RNA-seq incorporating quantification uncertainty. Nature methods 14, 687-690 (2017).
51. Yu, G., Wang, L. G., Han, Y. & He, Q. Y. ClusterProfiler: An R package for comparing biological themes among gene clusters. OMICS A Journal of Integrative Biology 16, 284-287 (2012).
52. Sergushichev, A. A. An algorithm for fast preranked gene set enrichment analysis using cumulative statistic calculation. bioRxiv 060012 (2016). doi:10.1101/060012
53. Kubinak, J. L. et al. MyD88 signaling in T cells directs IgA-mediated control of the microbiota to promote health. Cell host & microbe 17, 153-63 (2015).
54. Plaine, A. et al. Functional analysis of Candida albicans GPI-anchored proteins: roles in cell wall integrity and caspofungin sensitivity. Fungal genetics and biology: FG & B 45, 1404-14 (2008).
55. Castano, I. et al. Telomere length control and transcriptional regulation of subtelomeric adhesins in Candida glabrata. Molecular Microbiology 55, 1246-1258 (2005).
56. Vyas, V. K. et al. New CRISPR Mutagenesis Strategies Reveal Variation in Repair Mechanisms among Fungi. mSphere 3, (2018).
57. Veri, A. O. et al. Tuning Hsfl levels drives distinct fungal morphogenetic programs with depletion impairing Hsp90 function and overexpression expanding the target space. PLOS Genetics 14, e1007270 (2018).

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

### EMBODIMENTS

In an aspect is provided a method of ameliorating and/or preventing an intestinal disease in a mammal including administering to the mammal an immunogenic amount of a vaccine including a Candida adhesin polypeptide, or an immunogenic fragment thereof, in a pharmaceutically acceptable medium.

In some embodiments, the polypeptide includes an isolated agglutinin-like sequence (Als) protein, or an immunogenic fragment thereof.

In some embodiments, the polypeptide includes a protein selected from the group consisting of Als1, or an immunogenic fragment thereof, Als3, or an immunogenic fragment thereof, HYR1, or an immunogenic fragment thereof, and HWP1, or an immunogenic fragment thereof.

In some embodiments, the Als protein is selected from the group consisting of a Candida albicans Als3 protein and a Candida albicans Als1 protein, or an immunogenic fragment thereof.

In some embodiments, the Als protein includes the N-terminal domain of Candida albicans Als3 protein, or an immunogenic fragment thereof.

In some embodiments, the Candida adhesin polypeptide is derived from Candida strain selected from the group consisting of Candida albicans, Candida krusei, Candida tropicalis, Candida glabrata, Candida parapsilosis and Candida auris.

In some embodiments, the mammal is human.

In some embodiments, the intestinal disease is inflammatory bowel disease (IBD).

In some embodiments, the intestinal disease is Crohn's disease or colitis.

In some embodiments, the vaccine is administered by intramuscular, subcutaneous, intradermal, oral, or sublingual administration, or is administered for inhalation in a microparticulate formulation.

In some embodiments, the administering further includes administering a booster dose.

In some embodiments, the vaccine includes an immunostimulating adjuvant.

In another aspect is provided a vaccine including a Candida adhesin polypeptide, or an immunogenic fragment thereof, for use in a method of ameliorating and/or preventing an intestinal disease in a mammal.

In some embodiments, the polypeptide includes an isolated agglutinin-like sequence (Als) protein, or an immunogenic fragment thereof.

In some embodiments, the polypeptide includes a protein selected from the group consisting of Als1, or an immunogenic fragment thereof, Als3, or an immunogenic fragment thereof, HYR1, and HWP1, or an immunogenic fragment thereof.

In some embodiments, the Als protein is selected from the group consisting of a Candida albicans Als3 protein and a Candida albicans Als1 protein, or an immunogenic fragment thereof.

In some embodiments, the Als protein comprises the N-terminal domain of Candida albicans Als3 protein, or an immunogenic fragment thereof.

In some embodiments, the Candida adhesin polypeptide is derived from Candida strain selected from the group consisting of Candida albicans, Candida krusei, Candida tropicalis, Candida glabrata, Candida parapsilosis and Candida auris.

In some embodiments, the mammal is human.

In some embodiments, the intestinal disease is inflammatory bowel disease (IBD).

In some embodiments, the intestinal disease is Crohn's disease or colitis.

In some embodiments, the vaccine is administered as a pharmaceutical composition.

In some embodiments, the vaccine is administered by intramuscular, subcutaneous, intradermal, oral, or sublingual administration, or is administered for inhalation in a microparticulate formulation.

In some embodiments, the vaccine includes an immunostimulating adjuvant, and one or more pharmaceutically acceptable carriers or excipients.

What is claimed is:

1. A method of ameliorating and/or preventing an intestinal disease in a mammal comprising administering to the mammal an immunogenic amount of a vaccine comprising a Candida adhesin polypeptide, or an immunogenic fragment thereof, in a pharmaceutically acceptable medium.

2. The method of claim 1, wherein the polypeptide comprises an isolated agglutinin-like sequence (Als) protein, or an immunogenic fragment thereof.

3. The method of claim 1, wherein the polypeptide comprises a protein selected from the group consisting of Als1, or an immunogenic fragment thereof, Als3, or an immunogenic fragment thereof, HYR1, or an immunogenic fragment thereof, and HWP1, or an immunogenic fragment thereof.

4. The method of claim 2, wherein the Als protein is selected from the group consisting of a Candida albicans Als3 protein and a Candida albicans Als1 protein, or an immunogenic fragment thereof.

5. The method of claim 2, wherein the Als protein comprises the N-terminal domain of Candida albicans Als3 protein, or an immunogenic fragment thereof.

6. The method of claim 1, wherein the Candida adhesin polypeptide is derived from Candida strain selected from the group consisting of Candida albicans, Candida krusei, Candida tropicalis, Candida glabrata, Candida parapsilosis and Candida auris.

7. The method of claim 1, wherein the mammal is human.

8. The method of claim 1, wherein the intestinal disease is inflammatory bowel disease (IBD).

9. The method of claim 1, wherein the intestinal disease is Crohn's disease or colitis.

10. The method of claim 1, wherein the vaccine is administered by intramuscular, subcutaneous, intradermal, oral, or sublingual administration, or is administered for inhalation in a microparticulate formulation.

11. The method of claim 1, wherein the administering further comprises administering a booster dose.

12. The method of claim 1, wherein the vaccine comprises an immunostimulating adjuvant.

13. A vaccine comprising a Candida adhesin polypeptide, or an immunogenic fragment thereof, for use in a method of ameliorating and/or preventing an intestinal disease in a mammal.

14. The vaccine for use according to claim 13, wherein the polypeptide comprising an isolated agglutinin-like sequence (Als) protein, or an immunogenic fragment thereof.

15. The vaccine for use according to claim 13, wherein the polypeptide comprising a protein selected from the group consisting of Als1, or an immunogenic fragment thereof, Als3, or an immunogenic fragment thereof, HYR1, or an immunogenic fragment thereof, and HWP1, or an immunogenic fragment thereof.

16. The vaccine for use according to claim 15, wherein the Als protein is selected from the group consisting of a Candida albicans Als3 protein and a Candida albicans Als1 protein, or an immunogenic fragment thereof.

17. The vaccine for use according to claim 14, wherein the Als protein comprises the N-terminal domain of Candida albicans Als3 protein, or an immunogenic fragment thereof.

18. The vaccine for use according to claim 13, wherein the Candida adhesin polypeptide derived from Candida strain selected from the group consisting of Candida albicans, Candida krusei, Candida tropicalis, Candida glabrata, Candida parapsilosis and Candida auris.

19. The vaccine for use according to claim 13, wherein the mammal is human.

20. The vaccine for use according to claim 13, wherein the intestinal disease is inflammatory bowel disease (IBD).

21. The vaccine for use according to claim 13, wherein the intestinal disease is Crohn's disease or colitis.

22. The vaccine for use according to claim 13, wherein the vaccine is administered as a pharmaceutical composition.

23. The vaccine for use according to claim 13, wherein the vaccine is administered by intramuscular, subcutaneous, intradermal, oral, or sublingual administration, or is administered for inhalation in a microparticulate formulation.

24. The vaccine for use according to claim 13, wherein the vaccine comprises an immunostimulating adjuvant, and one or more pharmaceutically acceptable carriers or excipients.


SEQUENCE LISTING (63 sequences)

SEQ ID NO: 1 - DNA, 28 bp, Artificial Sequence
  Synthetic construct
atctcattagatttggaacttgtgggtt

SEQ ID NO: 2 - DNA, 22 bp, Artificial Sequence
  Synthetic construct
ttcgagcgtcccaaaaccttct

SEQ ID NO: 3 - DNA, 20 bp, Artificial Sequence
  Synthetic construct
gactgtcaaggagggtattc

SEQ ID NO: 4 - DNA, 20 bp, Artificial Sequence
  Synthetic construct
gaataccacttgtttaccgg

SEQ ID NO: 5 - DNA, 20 bp, Artificial Sequence
  Synthetic construct
ccgcaagtgattagacttag

SEQ ID NO: 6 - DNA, 23 bp, Artificial Sequence
  Synthetic construct
gaagttcctattctctagaaagt

SEQ ID NO: 7 - DNA, 45 bp, Artificial Sequence
  Synthetic construct
gctgcgctttattgttgaataaattaaaaatagtttacgcaagtc

SEQ ID NO: 8 - DNA, 43 bp, Artificial Sequence
  Synthetic construct
attcaacaataaagcgcagcgttttagagctagaaatagcaag

SEQ ID NO: 9 - DNA, 20 bp, Artificial Sequence
  Synthetic construct
cctacctactacttcctgtc

SEQ ID NO: 10 - DNA, 20 bp, Artificial Sequence
  Synthetic construct
gggtgtggattgaggcattg

SEQ ID NO: 11 - DNA, 80 bp, Artificial Sequence
  Synthetic construct
acatataattctttcatattttcattttatttcatacgttaagatccatatccaatagtcgggccccccctcgaggaagt

SEQ ID NO: 12 - DNA, 80 bp, Artificial Sequence
  Synthetic construct
ctatatctcaaagcgtggaaatatattcccactcgtccaaagtatatagatgtgaatttacgacaaggtgctgaaccaaa

SEQ ID NO: 13 - DNA, 21 bp, Artificial Sequence
  Synthetic construct
gaggctctttcctcctctcaa

SEQ ID NO: 14 - DNA, 20 bp, Artificial Sequence
  Synthetic construct
acccaaaacagcattccaag

SEQ ID NO: 15 - DNA, 45 bp, Artificial Sequence
  Synthetic construct
ctcaattgaaatgtgaaagtaaattaaaaatagtttacgcaagtc

SEQ ID NO: 16 - DNA, 43 bp, Artificial Sequence
  Synthetic construct
actttcacatttcaattgaggttttagagctagaaatagcaag

SEQ ID NO: 17 - DNA, 90 bp, Artificial Sequence
  Synthetic construct
attctatgtggtaaaagcatggactaaattttcaagttgagaataaatcatgcataaaggaagcatctctgcacaggaaacagctatgac

SEQ ID NO: 18 - DNA, 90 bp, Artificial Sequence
  Synthetic construct
acaatgtaaattgttgaagcatctgatattaacaattggtagttgtttgaacaattctgatgcgactatttatatttgtatgtgtgtagg

SEQ ID NO: 19 - DNA, 20 bp, Artificial Sequence
  Synthetic construct
cgttccaatgaatccaaacc

SEQ ID NO: 20 - DNA, 20 bp, Artificial Sequence
  Synthetic construct
actgtcgcagttgcagaaga

SEQ ID NO: 21 - DNA, 20 bp, Artificial Sequence
  Synthetic construct
tggaagcttcatcgcctatc

SEQ ID NO: 22 - DNA, 20 bp, Artificial Sequence
  Synthetic construct
gcgattgagattggttggtt

SEQ ID NO: 23 - DNA, 20 bp, Artificial Sequence
  Synthetic construct
aacaacggttctggaagtgg

SEQ ID NO: 24 - DNA, 20 bp, Artificial Sequence
  Synthetic construct
cagtgtgagcaccggtattg

SEQ ID NO: 25 - DNA, 20 bp, Artificial Sequence
  Synthetic construct
agctccatcacctgctgttt

SEQ ID NO: 26 - DNA, 20 bp, Artificial Sequence
  Synthetic construct
ctgaggtgcctgttgtcaag

SEQ ID NO: 27 - DNA, 20 bp, Artificial Sequence
  Synthetic construct
tggtccaggtgcttcttctt

SEQ ID NO: 28 - DNA, 20 bp, Artificial Sequence
  Synthetic construct
ggttgcatgagtggaactga

SEQ ID NO: 29 - DNA, 20 bp, Artificial Sequence
  Synthetic construct
gctgccaacaatttggttct

SEQ ID NO: 30 - DNA, 20 bp, Artificial Sequence
  Synthetic construct
tgatggcattgctacccata

SEQ ID NO: 31 - DNA, 22 bp, Artificial Sequence
  Synthetic construct
gtggtactaccatgttcccagg

SEQ ID NO: 32 - DNA, 25 bp, Artificial Sequence
  Synthetic construct
gatagaaccaccaatccagacagag

SEQ ID NO: 33 - DNA, 53 bp, Artificial Sequence
  Synthetic construct
cggtacccggggatctagaaagcatactatactattcgacacttcctttcaat

SEQ ID NO: 34 - DNA, 35 bp, Artificial Sequence
  Synthetic construct
gaaaagctgtttagacattggctcttcattgagct

SEQ ID NO: 35 - DNA, 56 bp, Artificial Sequence
  Synthetic construct
tctaaacagcttttcaattcaattcatcattttttttttattcttttttttgattt

SEQ ID NO: 36 - DNA, 57 bp, Artificial Sequence
  Synthetic construct
ggaaagagttttctttccaattttttttttttcgtcattataaaaatcattacgacc

SEQ ID NO: 37 - DNA, 33 bp, Artificial Sequence
  Synthetic construct
aagaaaactctttccattgccttttctaaagcg

SEQ ID NO: 38 - DNA, 37 bp, Artificial Sequence
  Synthetic construct
cgactctagaggatcgcagaggttcttacccactggt

SEQ ID NO: 39 - DNA, 22 bp, Artificial Sequence
  Synthetic construct
tccttggctggcactgaactcg

SEQ ID NO: 40 - DNA, 30 bp, Artificial Sequence
  Synthetic construct
atcacatgaagtcaaatcaacttttctagc

SEQ ID NO: 41 - DNA, 22 bp, Artificial Sequence
  Synthetic construct
catgagtagctggcaatgaagc

SEQ ID NO: 42 - DNA, 89 bp, Artificial Sequence
  Synthetic construct
ctttctttatacatataattctttcatattttcattttatttcatacgttaagatccatatccaatagtcggaaacagctatgaccatg

SEQ ID NO: 43 - DNA, 90 bp, Artificial Sequence
  Synthetic construct
gtaacgcaaccagaacgtgtccggctgcgctttattgttgaatttagtttcttctttgccatcgactatttatatttgtatgtgtgtagg

SEQ ID NO: 44 - DNA, 20 bp, Artificial Sequence
  Synthetic construct
ggtgccgtgcaagtttctat

SEQ ID NO: 45 - DNA, 29 bp, Artificial Sequence
  Synthetic construct
gcggccgcgtttggttcagcaccttgtcg

SEQ ID NO: 46 - DNA, 20 bp, Artificial Sequence
  Synthetic construct
acaggttgttgttcatcgca

SEQ ID NO: 47 - DNA, 20 bp, Artificial Sequence
  Synthetic construct
cctacctactacttcctgtc

SEQ ID NO: 48 - DNA, 21 bp, Artificial Sequence
  Synthetic construct
gaggctctttcctcctctcaa

SEQ ID NO: 49 - DNA, 20 bp, Artificial Sequence
  Synthetic construct
acccaaaacagcattccaag

SEQ ID NO: 50 - DNA, 35 bp, Artificial Sequence; identical to SEQ ID NO: 34
  Synthetic construct

SEQ ID NO: 51 - DNA, 17 bp, Artificial Sequence
  Synthetic construct
ggtgctccagctagatc

SEQ ID NO: 52 - DNA, 56 bp, Artificial Sequence; identical to SEQ ID NO: 35
  Synthetic construct

SEQ ID NO: 53 - DNA, 57 bp, Artificial Sequence; identical to SEQ ID NO: 36
  Synthetic construct

SEQ ID NO: 54 - DNA, 33 bp, Artificial Sequence; identical to SEQ ID NO: 37
  Synthetic construct

SEQ ID NO: 55 - DNA, 37 bp, Artificial Sequence; identical to SEQ ID NO: 38
  Synthetic construct

SEQ ID NO: 56 - DNA, 34 bp, Artificial Sequence
  Synthetic construct
cctttagataatttgtcaccgtggtggaagtttt

SEQ ID NO: 57 - DNA, 31 bp, Artificial Sequence
  Synthetic construct
cggtacccggggatctccaagtggttgactg

SEQ ID NO: 58 - DNA, 39 bp, Artificial Sequence
  Synthetic construct
caaattatctaaaggtgaagaattattcactggtgttgt

SEQ ID NO: 59 - DNA, 30 bp, Artificial Sequence
  Synthetic construct
caaaagcttcacgcgtctcgagatatcgat

SEQ ID NO: 60 - DNA, 39 bp, Artificial Sequence
  Synthetic construct
cgcgtgaagcttttgattaagccttctagtccaaaaaac

SEQ ID NO: 61 - DNA, 35 bp, Artificial Sequence; identical to SEQ ID NO: 34
  Synthetic construct

SEQ ID NO: 62 - DNA, 46 bp, Artificial Sequence
  Synthetic construct
acagggtaatatgatgtatagtgcttgctgttcgatattgctagag

SEQ ID NO: 63 - DNA, 51 bp, Artificial Sequence
  Synthetic construct
ggcccgggatccgatattttatgatggaatgaatgggatgaatcatcaaac